(19) United States
(12) Patent Application Publication
(10) Pub. No.: US 2025/0368619 A1
(43) Pub. Date: Dec. 4, 2025

(54) SUBSTITUTED TETRAHYDROCYCLOHEPTA[E]INDOLE DERIVATIVES, PROCESSES FOR THEIR PREPARATION AND THERAPEUTIC USES THEREOF

(71) Applicant: SANOFI, Paris (FR)

(72) Inventors: Patrick BERNARDELLI, Paris (FR); Youssef El-Ahmad, Paris (FR); Frank HALLEY, Paris (FR); Frédéric PETIT, Paris (FR); Franck SLOWINSKI, Paris (FR); Corinne TERRIER

(21) Appl. No.: 19/108,849

(22) PCT Filed: Sep. 8, 2023
(86) PCT No.: PCT/EP2023/074704

§ 371 Date: Mar. 5, 2025

(30) Foreign Application Priority Data
Sep. 9, 2022 (EP) 22306338.9

(51) Int. Cl. C07D 401/14; A61K 31/404; A61K 31/4439; C07D 403/10; C07D 405/14

(52) U.S. Cl. CPC C07D 401/14; A61K 31/404; A61K 31/4439; C07D 403/10; C07D 405/14

(57) ABSTRACT

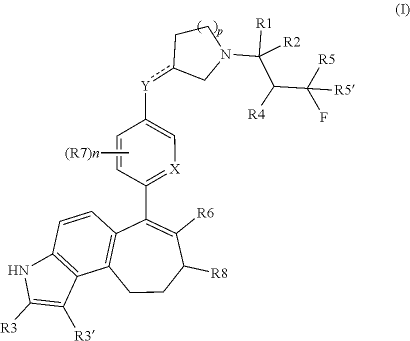

[symbol]

Disclosed herein are compounds of the formula (I) or a pharmaceutically acceptable salt thereof wherein R1 and R2 independently represent a hydrogen atom or a deuterium atom; R3 and R3′ represent a hydrogen atom, or a fluorine atom; R4 represents a hydrogen atom or a fluorine atom; R5 and R5′ independently represent a hydrogen atom or a fluorine atom; Y represents —CH2-, —CH═, —CR9=, —O— or —NH—; (AA) represents a single bond or a double bond; p is 0 or 1; X represents —CH═, —N═ or —CR″═; R6 represents a group selected from a phenyl group; a fused phenyl group; a phenyl group fused with a hetero(C₄-C₆)cycloalkyl; a bicyclic group comprising 5 to 12 carbon atoms; a heteroaryl group; a cycloalkyl group; a (C₃-C₆)cycloalkyl(C₁-C₃)alkyl group; a 4 to 7 membered-heterocycloalkyl group; a (C₁-C₆)alkyl group; a (C₁-C₆)alkenyl group; and a phenyl(C₁-C₂)alkyl group; R7 independently represents a (C₁-C₃)alkyl group, a halogen atom, a cyano group, or a (C₁-C₃)fluoroalkyl group; R8 represents a hydrogen atom or a (C₁-C₃)alkyl group or a cyclopropyl; and n is 0, 1 or 2. Further disclosed are process for preparing the same, pharmaceutical compositions comprising them as well as said compounds of formula (I) for use as an inhibitor and degrader of estrogen receptors, in particular in the treatment of ovulatory dysfunction, cancer, endometriosis, osteoporosis, benign prostatic hypertrophy or inflammation.

Disclosed herein are novel substituted tetrahydrocyclohepteneindole derivatives, the processes for their preparation, as well as the therapeutic uses thereof, in particular as anticancer agents via selective antagonism and degradation of estrogen receptors.

The Estrogen Receptors (ER) belong to the steroid/nuclear receptor superfamily involved in the regulation of eukaryotic gene expression, cellular proliferation and in target tissues. ERs are in two forms: the estrogen receptor alpha (ERα) and the estrogen receptor beta (ERβ) respectively encoded by the ESR1 and the ESR2 genes. ERα and ERβ are ligand-activated transcription factors which are activated by the hormone estrogen (the most potent estrogen produced in the body is 17β-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes.

ERα is mainly expressed in reproductive tissues such as uterus, ovary, breast, bone and white adipose tissue. Abnormal ERα signaling leads to development of a variety of diseases, such as cancers, metabolic and cardiovascular diseases, neurodegenerative diseases, inflammation diseases and osteoporosis.

ERα is expressed in not more than 10% of normal breast epithelium but approximately 50-80% of breast tumors. Such breast tumors with high level of ERα are classified as ERα-positive breast tumors. The etiological role of estrogen in breast cancer is well established and modulation of ERα signaling remains the mainstay of breast cancer treatment for the majority ERα-positive breast tumors. Currently, several strategies for inhibiting the estrogen axis in breast cancer exist, including: 1—blocking estrogen synthesis by aromatase inhibitors that are used to treat early and advanced ERα-positive breast cancer patients; 2—antagonizing estrogen ligand binding to ERα by tamoxifen which is used to treat ERα-positive breast cancer patients in both pre- and post-menopausal setting; 3—antagonizing and downregulating ERα levels by fulvestrant, which is used to treat breast cancer in patients that have progressed despite endocrine therapies such as tamoxifen or aromatase inhibitors.

Although these endocrine therapies have contributed enormously to reduction in breast cancer development, about more than one-third of ERα-positive patients display de novo resistance or develop resistance over time to such existing therapies. Several mechanisms have been described to explain resistance to such hormone therapies. For example, hypersensitivity of ERα to low estrogen level in treatment with aromatase inhibitors, the switch of tamoxifen effects from antagonist to agonist effects in tamoxifen treatments or multiple growth factor receptor signaling pathways. Acquired mutations in ERα occurring after initiation of hormone therapies may also play a role in treatment failure and cancer progression. Certain mutations in ERα, particularly those identified in the Ligand Binding Domain (LBD), result in the ability to bind to DNA in the absence of ligand and confer hormone independence in cells harboring such mutant receptors.

Most of the endocrine therapy resistance mechanisms identified rely on ERα-dependent activity. One of the new strategies to counterforce such resistance is to shut down the ERα signaling by removing ERα from the tumor cells using Selective Estrogen Receptors Degraders (SERDs). Clinical and preclinical data showed that a significant number of the resistance pathways can be circumvented by the use of SERDs.

There is still a need to provide SERDs with good degradation efficacy.

Documents WO2017/140669 and WO2018/091153 disclose some substituted 6,7-dihydro-5H-benzo[7]annulene compounds and substituted N-(3-fluoropropyl)-pyrrolidine derivatives useful as SERDs.

The inventors have now found novel compounds able to selectively antagonize and degrade the estrogen receptors (SERDs compounds), for use in cancer treatment.

Disclosed herein are compounds of the formula (I), or pharmaceutically acceptable salts thereof:

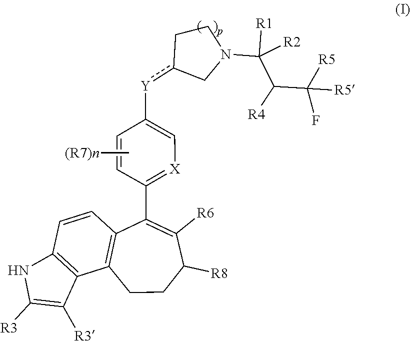

wherein:
R1 and R2 independently represent a hydrogen atom or a deuterium atom;
R3 and R3′ represent a hydrogen atom, or a fluorine atom;
R4 represents a hydrogen atom or a fluorine atom;
R5 and R5′ independently represent a hydrogen atom or a fluorine atom;
Y represents —CH₂—, —CH═, —CR9=, —O— or —NH—, wherein R9 represents a fluorine atom or a (C₁-C₃)alkyl group;
[symbol] represents a single bond or a double bond;
p is 0 or 1;
X represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl;
R6 represents a group selected from:
   a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; a —COOH group and a —OH group;
   a fused phenyl group, selected from phenyl groups fused with a (C₃-C₆)cycloalkyl, which (C₃-C₆)cycloalkyl ring optionally comprises an unsaturation and, wherein the fused phenyl group is optionally substituted with 1 to 3 substituents independently selected from a (C₁-C₃) alkyl group, a hydroxy group, a halogen atom, a (C₁-C₆)fluoroalkyl group and a (C₁-C₃)alkoxy group;
   a phenyl group fused with a hetero(C₄-C₆)cycloalkyl, which hetero(C₄-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
   a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)-alkyl group, a (C₁-C₃)fluoroalkyl group, a (C₁-C₃)alkoxy group, a (C₁-C₃)fluoroalkoxy group and an oxo group;
   a heteroaryl group comprising 2 to 9 carbon atoms and comprising from 1 to 3 heteroatoms independently selected from oxygen, nitrogen and sulfur, and at least 5 atoms including carbon atoms and heteroatoms, such as a pyridyl group, a pyridone group or a pyrrolyl group, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
   a cycloalkyl group comprising 3 to 7 carbon atoms, said cycloalkyl group being saturated or partially saturated and being optionally substituted with 1 to 4 substituents independently selected from:
      a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
      a (C₃-C₆)cycloalkyl group, and a phenyl group, said (C₃-C₆)cycloalkyl or phenyl groups being optionally substituted with one or two halogen atom(s) or (C₁-C₃)alkyl group(s);
   a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
   a 4 to 7 membered-heterocycloalkyl group comprising 1 or 2 heteroatoms independently selected from oxygen, nitrogen and sulfur, such as a tetrahydropyranyl, a dihydropyran or a tetrahydrofuranyl group, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
   a (C₁-C₆)alkyl group, such as an isobutyl group or an ethylbutyl group, said alkyl group being optionally substituted with 1 to 4 substituents independently selected from: a fluorine atom, a (C₁-C₃)alkoxy group, a (C₁-C₃)fluoroalkoxy group and a —OH group;
   a (C₁-C₆)alkenyl group, said (C₁-C₆)alkenyl group being optionally substituted with 1 to 4 substituents independently selected from: a —OH group; a halogen atom; a (C₁-C₃)alkyl group; a (C₁-C₃)fluoroalkyl group; a (C₁-C₃)alkoxy group; a (C₁-C₃) fluoroalkoxy group; a —COOH group and a cyano group; and
   a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃) fluoroalkoxy group; a cyano group; and a —OH group;
R7 independently represents a (C₁-C₃)alkyl group, such as a methyl group, a halogen atom, such as a fluorine atom, a cyano group, or a (C₁-C₃)fluoroalkyl group, such as a trifluoromethyl;
R8 represents a hydrogen atom or a (C₁-C₃)alkyl group or a cyclopropyl; and
n is 0, 1 or 2.

The compounds of formula (I) can contain one or more asymmetric carbon atoms. They may therefore exist in the form of enantiomers.

The compounds of formula (I) may be present as well under tautomer forms.

The compounds of formula (I) may exist in the form of bases, acids, zwitterion or of addition salts with acids or bases. Hence, herein are provided compounds of formula (I) or pharmaceutically acceptable salts thereof.

These salts may be prepared with pharmaceutically acceptable acids or bases, although the salts of other acids or bases useful, for example, for purifying or isolating the compounds of formula (I) are also provided.

Among suitable salts of the compounds of formula (I), trifluoroacetate may be cited.

As used herein, the terms below have the following definitions unless otherwise mentioned throughout the instant specification:
a halogen atom: a fluorine, a chlorine, a bromine or an iodine atom, and in particular a fluorine and a chlorine atom;
an oxo: a “═O” group;
an alkyl group: a linear or branched saturated hydrocarbon-based aliphatic group comprising, unless otherwise mentioned, from 1 to 6 carbon atoms (noted “(C₁-C₆)-alkyl”); By way of examples, mention may be made of, but not limited to: methyl, ethyl, propyl, isopropyl, butyl, isobutyl, sec-butyl, tert-butyl, pentyl, isopentyl, hexyl and isohexyl groups, and the like;
an alkenyl group: a linear or branched hydrocarbon-based aliphatic group comprising, unless otherwise mentioned, from 1 to 6 carbon atoms (noted “(C₁-C₆)-alkylene”) and at least an unsaturation. By way of examples, mention may be made of, but not limited to: vinyl group, and the like;
a cycloalkyl group: a monocyclic alkyl group comprising, unless otherwise mentioned, from 3 to 7 carbon atoms, saturated or partially unsaturated and unsubstituted or substituted. By way of examples, mention may be made of, but not limited to: cyclopropyl, cyclobutyl, cyclopentyl, cyclobutenyl, cyclopentenyl, cyclohexyl, cyclohexenyl, cycloheptyl, cycloheptenyl, groups and the like, in particular a cyclopentyl, a cyclohexyl, a cycloheptyl, a cycloheptenyl, or a cyclohexenyl;
a heterocycloalkyl group: a 4 to 7-membered cycloalkyl group, in particular a 4 to 6-membered cycloalkyl group, saturated or partially unsaturated, comprising 1 to 2 heteroatoms independently selected from oxygen, nitrogen and sulfur, in particular being oxygen or nitrogen. By way of examples, mention may be made of, but not limited to: morpholinyl, piperazinyl, piperidinyl, pyrrolidinyl, aziridinyl, oxanyl, oxetanyl, tetrahydropyranyl, morpholinyl, tetrahydrofuranyl, oxepanyl, diazepanyl, dioxanyl, dihydropyranyl,tetrahydropyranyl, and tetrahydrothiopyranyl. The heterocycloalkyl is advantageously tetrahydrofuranyl or tetrahydropyranyl;
a fluoroalkyl group: an alkyl group as previously defined where the alkyl group is substituted with at least one fluorine atom. In other terms, at least one hydrogen atom of the alkyl group is replaced by a fluorine atom. By way of example, mention may be made of —CH₂F, —CHF₂, CH₂CHF₂, —CH₂CH₂F and the like. When all the hydrogen atoms belonging to the alkyl group are replaced by fluorine atoms, the fluoroalkyl group can be named perfluoroalkyl group. By way of example, mention may be made of trifluoromethyl group or trifluoroethyl group and the like;
an alkoxy group: an —O-alkyl group where the alkyl group is as previously defined. By way of examples, mention may be made of, but not limited to: methoxy, ethoxy, propoxy, isopropoxy, linear, secondary or tertiary butoxy, isobutoxy, pentoxy or hexoxy groups, and the like;
a fluoroalkoxy group: an —O-alkyl group where the alkyl group is as previously defined and where the alkyl group is substituted with at least one fluorine atom. In other terms, at least one hydrogen atom of the alkyl group is replaced by a fluorine atom. By way of example, mention may be made of —OCH₂F, —OCHF₂, —OCH₂CH₂F and the like. When all the hydrogen atoms belonging to the alkyl group are replaced by fluorine atoms, the fluoroalkoxy group can be named perfluoroalkoxy group. By way of example, mention may be made of trifluoromethoxy group and the like;
a (C₁-C₄)alkylthio group also named a (C₁-C₄)alkylsulfanyl group: a —S-alkyl group where the alkyl group is as previously defined. By way of examples, mention may be made of, but not limited to: methylthio, ethylthio, propylthio, isopropylthio, linear, secondary or tertiary butylthio, isobutylthio, and the like;
a (C₁-C₄)alkylsulfonyl group: a —SO₂-alkyl group where the alkyl group is as previously defined. By way of examples, mention may be made of, but not limited to —SO₂CH₃, —SO₂CH₂CH₃ and the like;
a (C₁-C₄)fluoroalkylthio group also named a (C₁-C₄)fluoroalkylsulfanyl group: a —S-fluoroalkyl group where the fluoroalkyl group is as previously defined. By way of examples, mention may be made of, but not limited to: fluoromethylthio, difluoromethylthio, trifluoromethylthio and the like;
a fused phenyl group: a bicyclic radical comprising from 7 to 10 carbon atoms and that contains a phenyl moiety. Said phenyl moiety may be fused to a (C₃-C₆)cycloalkyl group, i.e. the phenyl moiety may share a bond with said (C₃-C₆)cycloalkyl group. The fused phenyl group may be bound to the rest of the molecule by its phenyl moiety. It may be substituted. Examples are, but are not limited to indanyl, bicyclo[4.2.0]octa-1(6),2,4-trienyl, tetrahydronaphthalenyl and the like;
a phenyl group fused with a hetero(C₄-C₆)cycloalkyl: a bicyclic radical comprising from 7 to 10 carbon atoms and that contains a phenyl moiety. Said phenyl moiety may be fused to a hetero(C₄-C₆)cycloalkyl group, i.e. the phenyl moiety may share a bond with said hetero(C₄-C₆)cycloalkyl group. The fused phenyl group may be bound to the rest of the molecule by its phenyl moiety. It may be substituted. Examples are, but are not limited to a chromanyl group, in particular a chroman-8-yl group and the like;
a heteroaryl group: a cyclic 5 to 10-membered aromatic group containing between 2 and 9 carbon atoms and containing between 1 and 3 heteroatoms, such as nitrogen, oxygen or sulfur. Such nitrogen atom may be substituted with an oxygen atom in order to form a —N—O bond. Such —N—O bond can be in a form of a N-oxide (—N⁺—O—). Said heteroaryl group may be monocyclic or bicyclic. By way of examples of heteroaryl groups, mention may be made of, but not limited to: thiophene, furan, thiadiazole, thiazole, imidazole, pyridazine, triazine, pyrazine, oxadiazole, pyrazole, isothiazole, oxazole, isoxazole, pyridine, pyrimidine, benzotriazole, benzoxazole, pyrrolo[2,3-b]pyridine, benzimidazole, benzoxadiazole, benzothiazole, benzothiadiazole, benzofuran, indole, isoquinoline, indazole, benzisoxazole, benzisothiazole, pyridone groups and the like. The heteroaryl group is advantageously pyridine, pyrrole, imidazole, pyrazine, furane, thiazole, pyrazole, thiadiazole, pyridazine, pyridone and pyrimidine, and more particularly pyridine, pyridone and pyrrole;
a bicyclic group, generally comprising 5 to 12 carbon atoms, is a hydrocarbon group selected from groups comprising two rings connected through:
   a single common atom: a “spirobicyclic ring”. Such spiro bicyclic alkyl generally comprises 5 to 11 carbon atoms referring to a “spiro(C₅-C₁₁)bicyclic ring”. The rings may be saturated or partially unsaturated. Such spirobicyclic ring may be unsubstituted or substituted, in particular by at least one (C₁-C₃)alkyl group such as methyl or a fluorine. By way of examples of spiro(C₅-C₁₁)bicyclic ring as for the definition of R6, mention may be made of, but not limited to: spiro[2.3]hexane, spiro[3.3]heptane, spiro[3.3]heptene, spiro[2.5]octane and 7-azaspiro[3.5]nonane. The spiro(C₅-C₁₁)bicyclic ring is advantageously spiro[3.3]heptane or spiro[3.3]heptene still for the R6 group;
   two common atoms. In that case the bicyclic group comprises 7 to 12 carbon atoms and optionally comprises 1 to 2 unsaturations. By way of examples of such bicyclic groups, mention may be made of, but not limited to: cis-1,3a,4,5,6,6a-hexahydropentalenyl group, bicyclo[3.1.0]hexan-1-yl, bicyclo[4.1.0]heptanyl and octahydropentalenyl; or
   three or more common atoms. In that case the bicyclic group comprises 6 to 10 carbon atoms, such bicyclic group may be referred to as a “bridged (C₆-C₁₀)cycloalkyl” group, the rings share three or more atoms and the bridge contains at least one atom, for example 1, 2 or 3 atoms and preferentially 1 atom. By way of examples of such bridged cycloalkyl groups, mention may be made of, but not limited to bicyclo[3.2.1]octan-3-yl and bicyclo[2.2.1]heptan-2-yl;
A zwitterion means: a globally neutral molecule with a positive and a negative electrical charge and having an acidic group and a basic group.

In another embodiment, in the compounds of formula (I) as defined above, R1 and R2 are a hydrogen atom.

In another embodiment, in the compounds of formula (I) as defined above, R3 and R3′ are a hydrogen atom.

In another embodiment, in the compounds of formula (I) as defined above, R4, R5 and R5′ represent a hydrogen atom.

In another embodiment, in the compounds of formula (I) as defined above, X represents —CH═.

In another embodiment, in the compounds of formula (I) as defined above, Y is —CH₂—, —CH═, —O— or —NH—.

In another embodiment, in the compounds of formula (I) as defined above, R7 represents a hydrogen atom and n is 1.

In another embodiment, in the compounds of formula (I) as defined above, R6 represents a phenyl group, said phenyl group being optionally substituted by 1 to 3 substituents independently selected from a fluorine atom; a chlorine atom; a (C₁-C₄)alkyl group, such as a methyl or ethyl group, optionally substituted with a OH group; a trifluoromethyl group; a (C₁-C₄)alkoxy group, such as a methoxy group; a cyano group; a —COOH group and a —OH group.

In another embodiment, in the compounds of formula (I) as defined above, R6 represents a pyridyl group, said pyridyl group being optionally substituted with 1 to 3 substituents independently selected from a fluorine atom and a (C₁-C₆)alkoxy group, more particularly a methoxy group.

In another embodiment, in the compounds of formula (I) as defined above, R6 represents a saturated or partially saturated cyclohexyl group, said cyclohexyl group being optionally substituted with 1 to 2 fluorine atoms.

In another embodiment, in the compounds of formula (I) as defined above, R6 represents a saturated or partially saturated 6-membered heterocycloalkyl group comprising an oxygen atom, such as a dihydropyranyl.

In another embodiment, in the compounds of formula (I) as defined above, R8 represents a hydrogen atom.

Among the compounds of formula (I) described herein, mention may be made in particular of the following compounds or a pharmaceutically acceptable salt thereof, in particular hydrochloride salt thereof:
7-(4-fluoro-2-methylphenyl)-6-(4-((1-(3-fluoropropyl)azetidin-3-yl)methyl)phenyl)-3,8,9,10-tetrahydrocyclohepta[e]indole (1),
7-(3-fluoro-2-methoxypyridin-4-yl)-6-(4-((1-(3-fluoropropyl)azetidin-3-yl)methyl)phenyl)-3,8,9,10-tetrahydrocyclohepta[e]indole (2),
7-(3-chloro-2-methylphenyl)-6-(4-((1-(3-fluoropropyl)azetidin-3-ylidene)methyl)phenyl)-3,8,9,10-tetrahydrocyclohepta[e]indole (3),
(S)-7-(2,4-dichlorophenyl)-6-(4-((1-(3-fluoropropyl)pyrrolidin-3-yl)oxy)phenyl)-3,8,9,10-tetrahydrocyclohepta[e]indole (4),
(S)-7-(4,4-difluorocyclohex-1-en-1-yl)-6-(4-((1-(3-fluoropropyl)pyrrolidin-3-yl)oxy)phenyl)-3,8,9,10-tetrahydrocyclohepta[e]indole (5),
2,6-difluoro-3-(6-(4-((1-(3-fluoropropyl)azetidin-3-yl)methyl)phenyl)-3,8,9,10-tetrahydrocyclohepta[e]indol-7-yl)phenol (6),
7-(2,4-dichlorophenyl)-6-(4-((1-(3-fluoropropyl)azetidin-3-yl)methyl)phenyl)-3,8,9,10-tetrahydrocyclohepta[e]indole (7),
7-(3-chloro-2-methylphenyl)-6-(4-((1-(3-fluoropropyl)azetidin-3-yl)methyl)phenyl)-3,8,9,10-tetrahydrocyclohepta[e]indole (8),
7-(2,3-dimethoxyphenyl)-6-(4-((1-(3-fluoropropyl)azetidin-3-yl)methyl)phenyl)-3,8,9,10-tetrahydrocyclohepta[e]indole (9),
6-(4-((1-(3-fluoropropyl)azetidin-3-yl)methyl)phenyl)-7-(3-(trifluoromethyl)phenyl)-3,8,9,10-tetrahydrocyclohepta[e]indole (10),
3-(6-(4-((1-(3-fluoropropyl)azetidin-3-yl)methyl)phenyl)-3,8,9,10-tetrahydrocyclohepta[e]indol-7-yl)benzonitrile (11),
2-(6-(4-((1-(3-fluoropropyl)azetidin-3-yl)methyl)phenyl)-3,8,9,10-tetrahydrocyclohepta[e]indol-7-yl)benzonitrile (12),
4-(6-(4-((1-(3-fluoropropyl)azetidin-3-yl)methyl)phenyl)-3,8,9,10-tetrahydrocyclohepta[e]indol-7-yl)benzonitrile (13),
6-(4-((1-(3-fluoropropyl)azetidin-3-yl)methyl)phenyl)-7-(2-methoxypyridin-4-yl)-3,8,9,10-tetrahydrocyclohepta[e]indole (14),
6-(4-((1-(3-fluoropropyl)azetidin-3-yl)methyl)phenyl)-7-(6-methoxypyridin-3-yl)-3,8,9,10-tetrahydrocyclohepta[e]indole (15)
7-(3,6-dihydro-2H-pyran-4-yl)-6-(4-((1-(3-fluoropropyl)azetidin-3-yl)methyl)phenyl)-3,8,9,10-tetrahydrocyclohepta[e]indole (16),
(E)-3-(6-(4-((1-(3-fluoropropyl)azetidin-3-yl)methyl)phenyl)-3,8,9,10-tetrahydrocyclohepta[e]indol-7-yl)prop-2-en-1-ol (17),
(E)-4-(6-(4-((1-(3-fluoropropyl)azetidin-3-yl)methyl)phenyl)-3,8,9,10-tetrahydrocyclohepta[e]indol-7-yl)-2-methylbut-3-en-2-ol (18),
(3-(6-(4-((1-(3-fluoropropyl)azetidin-3-yl)methyl)phenyl)-3,8,9,10-tetrahydrocyclohepta[e]indol-7-yl)phenyl)methanol (19),
(4-(6-(4-((1-(3-fluoropropyl)azetidin-3-yl)methyl)phenyl)-3,8,9,10-tetrahydrocyclohepta[e]indol-7-yl)phenyl)methanol (20),
2-(3-(6-(4-((1-(3-fluoropropyl)azetidin-3-yl)methyl)phenyl)-3,8,9,10-tetrahydrocyclohepta[e]indol-7-yl)phenyl)ethan-1-ol (21),
3-(6-(4-((1-(3-fluoropropyl)azetidin-3-yl)methyl)phenyl)-3,8,9,10-tetrahydrocyclohepta[e]indol-7-yl)benzoic acid (22),
4-(6-(4-((1-(3-fluoropropyl)azetidin-3-yl)methyl)phenyl)-3,8,9,10-tetrahydrocyclohepta[e]indol-7-yl)benzoic acid, 2,2,2-trifluoroacetic acid (23),
(E)-3-(6-(4-((1-(3-fluoropropyl)azetidin-3-yl)methyl)phenyl)-3,8,9,10-tetrahydrocyclohepta[e]indol-7-yl)acrylic acid (24),
3-(6-(4-((1-(3-fluoropropyl)azetidin-3-yl)methyl)phenyl)-3,8,9,10-tetrahydrocyclohepta[e]indol-7-yl)propan-1-ol (25),
7-(3-chloro-2-methylphenyl)-6-(4-((1-(3-fluoropropyl)azetidin-3-yl)oxy)phenyl)-3,8,9,10-tetrahydrocyclohepta[e]indole (26),
N-(4-(7-(3-chloro-2-methylphenyl)-3,8,9,10-tetrahydrocyclohepta[e]indol-6-yl)phenyl)-1-(3-fluoropropyl)azetidin-3-amine (27),
(S)—N-(4-(7-(3-chloro-2-methylphenyl)-3,8,9,10-tetrahydrocyclohepta[e]indol-6-yl)phenyl)-1-(3-fluoropropyl)pyrrolidin-3-amine (28), and
(Z)-7-(3-chloro-2-methylphenyl)-6-(4-((1-(3-fluoropropyl)pyrrolidin-3-ylidene)methyl)phenyl)-3,8,9,10-tetrahydrocyclohepta[e]indole (29).

Another embodiment is a compound selected from the above list, or a pharmaceutically acceptable salt thereof, for use in therapy, especially as an inhibitor and degrader of estrogen receptors.

Another embodiment is a compound selected from the above list, or a pharmaceutically acceptable salt thereof, for use in the treatment of cancer, especially breast cancer.

Another embodiment is a method of inhibiting and degrading estrogen receptors, comprising administering to a subject in need thereof, in particular a human, a therapeutically effective amount of a compound selected from the above list, or a pharmaceutically acceptable salt thereof.

Another embodiment is a method of treating ovulatory dysfunction, cancer, endometriosis, osteoporosis, benign prostatic hypertrophy or inflammation, comprising administering to a subject in need thereof, in particular a human, a therapeutically effective amount of a compound selected from the above list, or a pharmaceutically acceptable salt thereof.

Another embodiment is a method of treating cancer, comprising administering to a subject in need thereof, in particular a human, a therapeutically effective amount of a compound selected from the above list, or a pharmaceutically acceptable salt thereof.

Another embodiment is a pharmaceutical composition comprising as active principle an effective dose of a compound selected from the above list, or a pharmaceutically acceptable salt thereof, and also at least one pharmaceutically acceptable excipient.

The compounds of the formula (I) can be prepared by the following processes.

The compounds of the formula (I) and other related compounds having different substituents are synthesized using techniques and materials described below or otherwise known by the skilled person in the art. In addition, solvents, temperatures and other reaction conditions presented below may vary as deemed appropriate to the skilled person in the art.

General below methods for the preparation of compounds of formula (I) optionally modified by the use of appropriate reagents and conditions for the introduction of the various moieties found in the formula (I) are described below.

The following abbreviations and empirical formulae are used:
MeCN Acetonitrile
NH₄Cl Ammonium chloride
Pd(dppf)Cl₂ [1,1′-Bis(diphenylphosphino)ferrocene]dichloropalladium(II)
CO₂ Carbon dioxide
Cs₂CO₃ Cesium carbonate
DBU 1,8-Diazabicyclo[5.4.0]undec-7-ene
DCM Dichloromethane
Et₂O Diethyl ether
DMF N,N-dimethylformamide
DMSO Dimethyl sulfoxide
Xantphos (9,9-Dimethyl-9H-xanthene-4,5-diyl)bis(diphenylphosphane)
EtOH Ethanol
EtOAc Ethyl acetate
H₂ Hydrogen
HCl Hydrochloric acid
LiHMDS Lithium bis(trimethylsilyl)amide
LiOH Lithium hydroxide
MeOH Methanol
MgSO₄ Magnesium sulfate
2-MeTHF 2-Methyltetrahydrofuran
Pd/C Palladium on carbon
Pd(OAc)₂ Palladium acetate
Pd(PPh₃)₂Cl₂ Palladium(II)bis(triphenylphosphine) dichloride
PtO₂ Platinum oxide
AcOK Potassium acetate
KHMDS Potassium bis(trimethylsilyl)amide
K₂CO₃ Potassium carbonate
KOH Potassium hydroxide
PG Protecting group
NaHCO₃Sodium bicarbonate
NaBH₄ Sodium borohydride
NaH Sodium hydride
NaOH Sodium hydroxide
Na₂SO₄ Sodium sulfate
SCX Strong cation exchange
SFC Supercritical Fluid Chromatography
TEA Triethylamine
TFA Trifluoroacetic acid
THF Tetrahydrofuran
Pd₂(dba)₃ tris(dibenzylideneacetone)dipalladium(0)
RT Room temperature

### SCHEME 1a Parts 1 and 2: Preparation of Compounds of Formula (I)—General Process

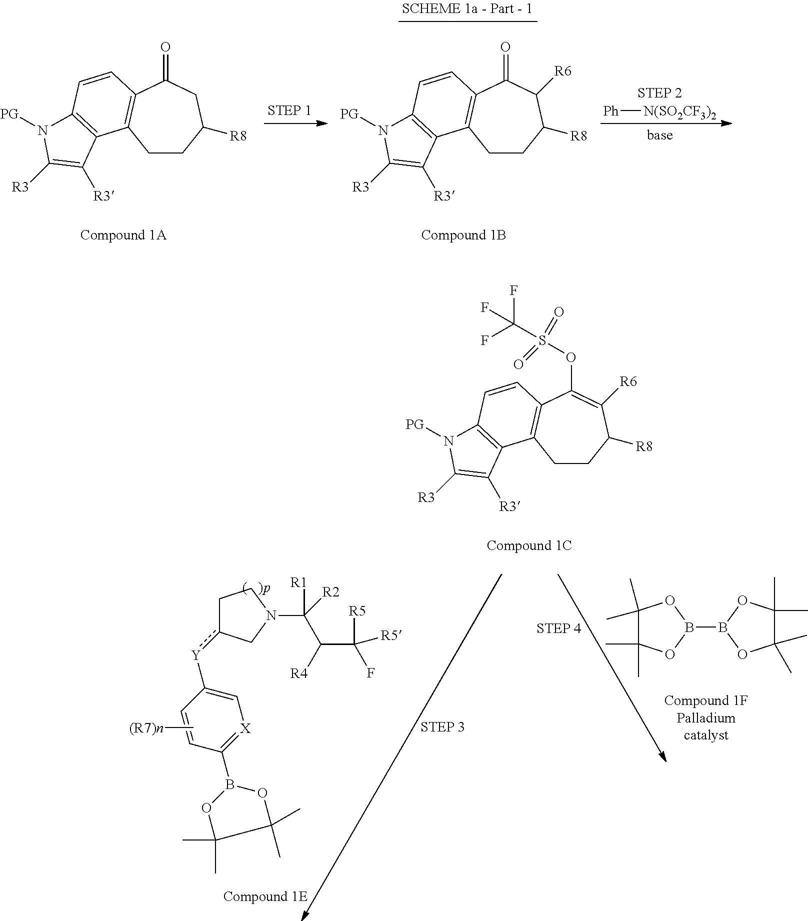

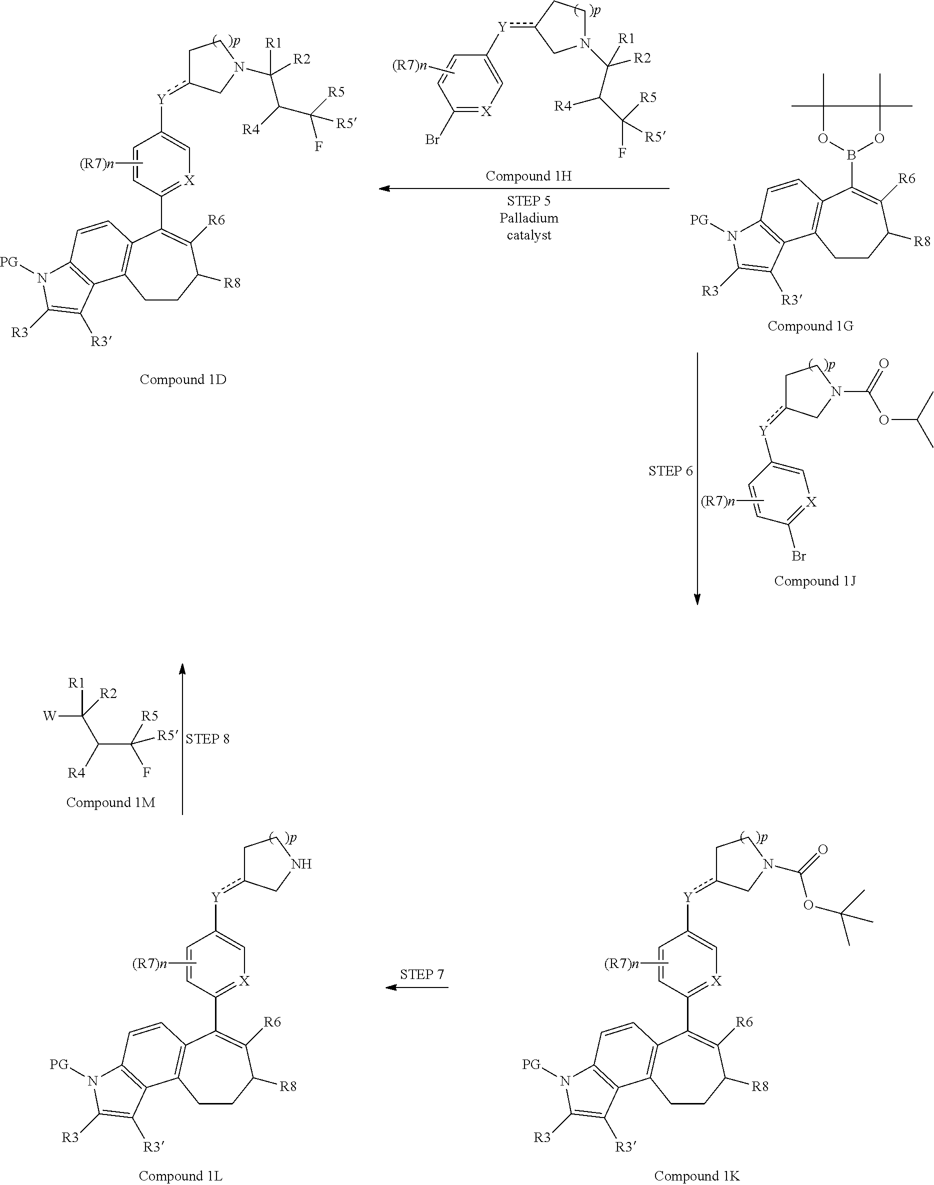

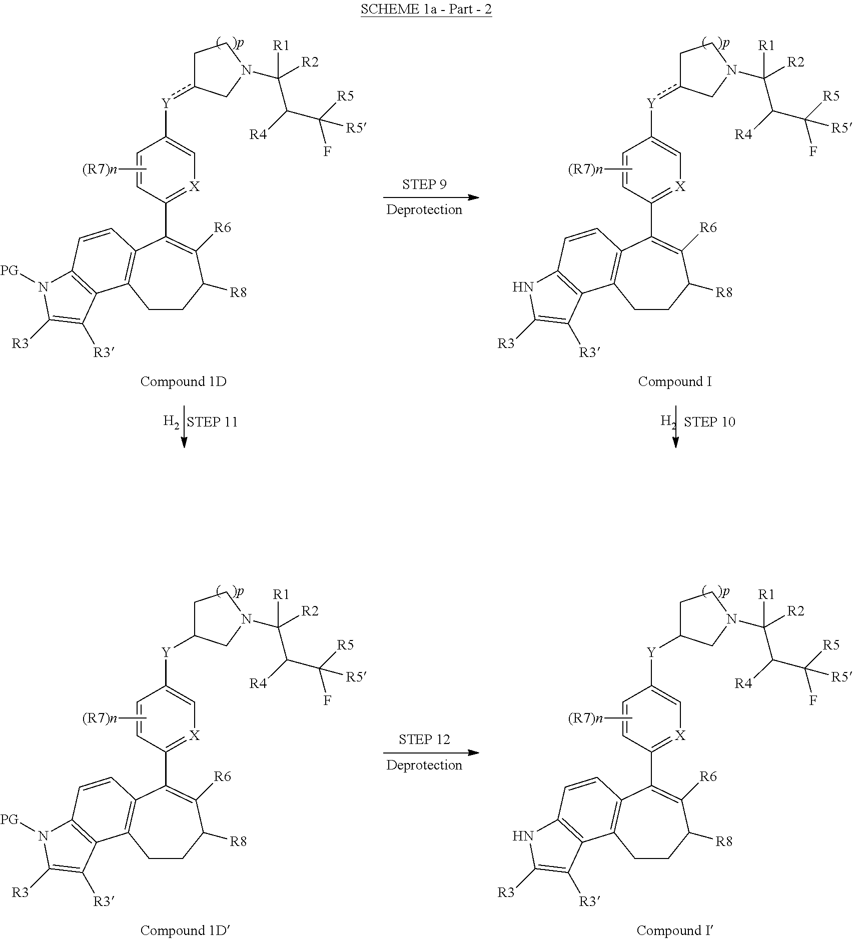

According to SCHEME 1a—Part 1 and Part 2, in which R1, R2, R3, R3′, R4, R5, R5′, R6, R7, R8, n, p, X, [symbol] and Y are as defined above and PG is a protecting group (PG) such as a tosyl group, a benzenesulfonamide group, a methoxymethylamine, a ethoxymethylamine or a 1-adamantyl carbamate group, compound 1A can be converted in STEP 1 to compound 1B by treatment with aryl or heteroaryl bromide or iodide in the presence of a palladium catalyst, for example tris(dibenzylideneacetone)dipalladium(0) Pd₂(dba)₃, and a phosphine such as (9,9-dimethyl-9H-xanthene-4,5-diyl)bis(diphenylphosphane) (XANTPHOS) in solution in toluene by heating up to reflux of solvent, in presence of a base such as K₂CO₃ or Cs₂CO₃. Alternative way to prepare compound 1B, wherein R6 can be any of the groups defined above for R6 in formula (I), is described in SCHEME 1g below.

Compound 1B can be converted in STEP 2 to compound 1C by treatment with N,N-bis(trifluoromethylsulfonyl)aniline in the presence of a base such as DBU or NaH, or KHMDS, in a solvent such as 2-MeTHF.

Compound 1C can be converted in STEP 4 to compound 1G by treatment for example with Compound 1F, and with a palladium catalyst, for example bis (triphenylphosphine) palladium(II) dichloride Pd(PPh₃)₂Cl₂, and a phosphine, such as triphenylphosphine, in solution in toluene by heating up to reflux of solvent, in presence of a base such as KOPh.

Compound 1D can be prepared in a Suzuki coupling reaction either between compounds 1C and 1E in STEP 3 or between compounds 1G and 1H in STEP 5 using a catalyst, for example [1,1′-bis(diphenylphosphino) ferrocene]dichloropalladium(II) (Pd(dppf)Cl₂) complex with DCM, in a solvent, such as a mixture of dioxane and water, and in the presence of a base, for example cesium carbonate (Cs₂CO₃), by heating up to reflux of solvent.

Alternatively, compound 1G can be converted in STEP 6 to compound 1K in a Suzuki coupling reaction with compound 1J using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent. Compound 1K can be converted in STEP 7 to compound 1L by treatment with TFA in solution in DCM or HCl in solution in dioxane. Compound 1L can be converted in STEP 8 to compound 1D by treatment with compound 1M, wherein W is Br, I or OSO₂R with R═CH₃, PhMe, CF₃ or CF₂CF₂CF₂CF₃, in presence of a base such as potassium carbonate in DMF at 70° C. or in presence of sodium hydroxide or potassium hydroxide in THF at room temperature or in presence of aqueous sodium hydroxide in DCM at room temperature.

PG of compound 1D can be deprotected into compound I in STEP 9 using methods known in the litterature (Protective Groups in Organic Synthesis, Theodora W. Greene, Peter G. M. Wuts, John Wiley & Sons Inc). In particular when PG is a tosyl group or a benzenesulfonamide group the deprotection can be done by a treatment with an aqueous solution of potassium hydroxide in methanol for example. When PG is a methoxymethylamine group or an ethoxymethylamine group the deprotection can be performed in the presence of aqueous HCl for example. When PG is an adamantyl carbamate group the deprotection can be realized by a treatment with by aqueous NaOH for example.

When Y═CH, compound I may be reduced by hydrogenation in STEP 10 with a catalyst, such as Pd/C or platinum oxide (PtO₂) under hydrogen (H₂) pressure to give the corresponding saturated compound I′.

Alternatively, when Y═CH, compound I′ can be prepared by hydrogenation of compound 1D in STEP 11 with a catalyst, such as Pd/C or platinum oxide (PtO₂) under hydrogen (H₂) pressure followed by protecting group deprotection of compound 1D′ using the appropiate conditions cited above depending on PG.

### SCHEME 1b Parts 1 and 2: Preparation of Compounds of Formula (I′)—General Process

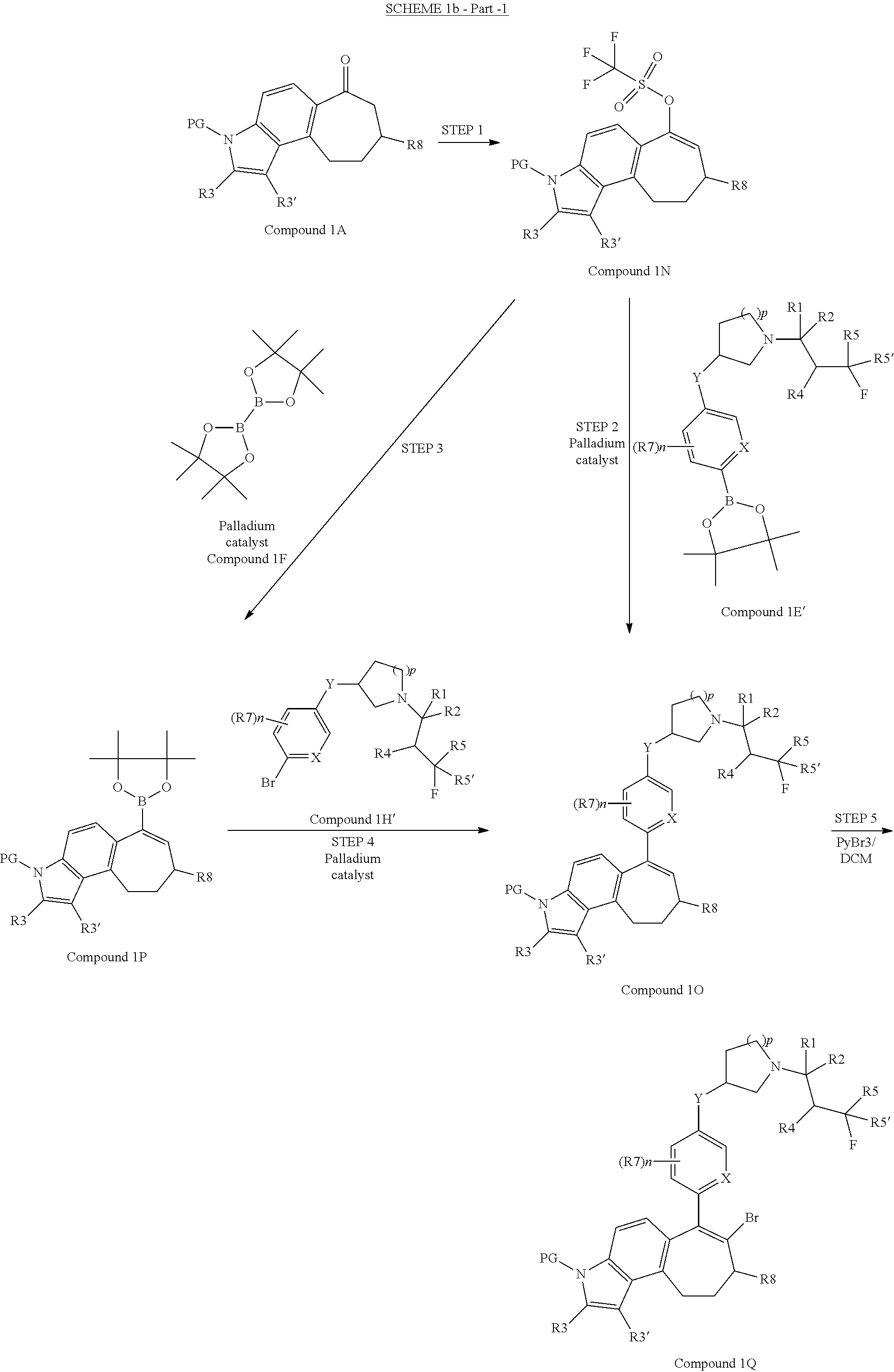

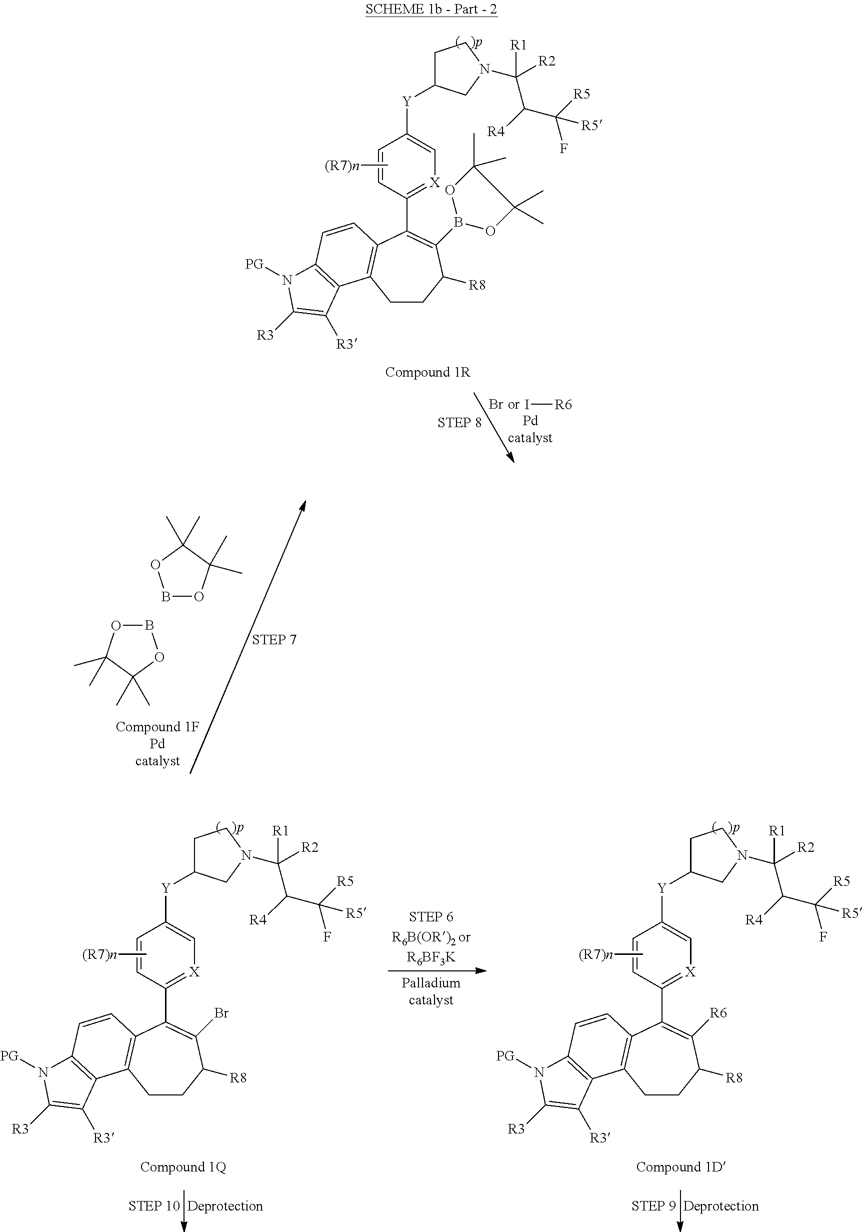

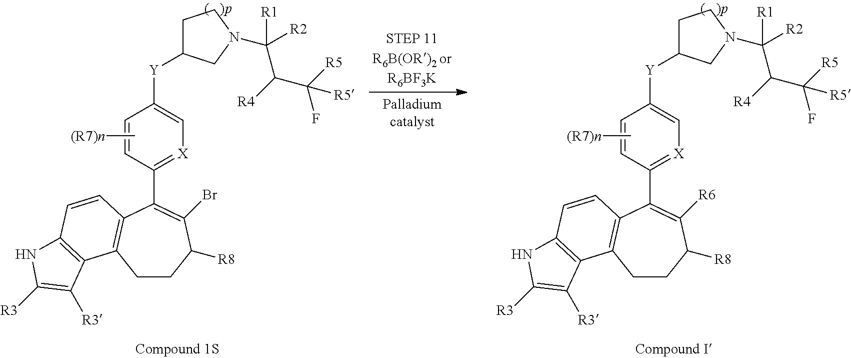

According to SCHEME 1b—Part 1 and Part 2, in which R1, R2, R3, R3′, R4, R5, R5′, R6, R7, R8, n, p, X and Y are as defined above and PG is a protecting group (PG) such as a tosyl group, a benzenesulfonamide group, a t-butyl carbamate group, a methoxymethylamine group, a ethoxymethylamine group or a 1-adamantyl carbamate group, compound 1A can be converted in STEP 1 to compound 1N by treatment with trifluoromethanesulfonic anhydride, in solution in DCM, in the presence of pyridine as a base.

Compound 1N can be converted in STEP 3 to compound 1P by treatment for example with Compound 1F, and with a palladium catalyst, for example bis (triphenylphosphine) palladium(II) dichloride Pd(PPh₃)₂Cl₂, and a phosphine, such as triphenylphosphine, in solution in toluene by heating up to reflux of solvent, in presence of a base such as KOPh.

Compound 1O can be prepared in a Suzuki coupling reaction either between compounds 1P and 1H′ in STEP 4 or between compounds 1N and 1E′ in STEP 2 using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1O can be converted in STEP 5 to compound 1Q by treatment for example with pyridinium tribromide in DCM or THF at room temperature.

Compound 1Q can be converted in STEP 7 to compound 1R by treatment for example with Compound 1F, and with a palladium catalyst, for example bis (triphenylphosphine) palladium(II) dichloride Pd(PPh₃)₂Cl₂, and a phosphine, such as triphenylphosphine, in solution in toluene by heating up to reflux of solvent, in presence of a base such as KOPh.

Compound 1D′ can be prepared in a Suzuki coupling reaction either between compounds 1Q and with a suitable boronic reagent R6B(OR′)₂, wherein —B(OR′)₂ is a boronic acid or a pinacolate ester and R6 is as defined above in STEP 6 or between compounds 1R and either R6Br or R6I or R6OTf and R6 is as defined above in STEP 8 using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

PG of compound 1D′ can be deprotected into compound I′ in STEP 9 using methods known in the litterature (Protective Groups in Organic Synthesis, Theodora W. Greene, Peter G. M. Wuts, John Wiley & Sons Inc). In particular when PG is a tosyl group or a benzenesulfonamide group the deprotection can be done by a treatment with an aqueous solution of potassium hydroxide in methanol for example. When PG is a t-butyl carbamate group, the deprotection can be performed in the presence of aqueous TFA for example. When PG is a methoxymethylamine group or an ethoxymethylamine group the deprotection can be performed in the presence of aqueous HCl for example. When PG is an adamantyl carbamate group the deprotection can be realized by a treatment with by aqueous NaOH for example.

Alternatively, PG of compound 1Q can be deprotected into compound 1S in STEP 10 using methods known in the litterature (Protective Groups in Organic Synthesis, Theodora W. Greene, Peter G. M. Wuts, John Wiley & Sons Inc). In particular when PG is a tosyl group or a benzenesulfonamide group the deprotection can be done by a treatment with an aqueous solution of potassium hydroxide in methanol for example. When PG is a t-butyl carbamate group, the deprotection can be performed in the presence of aqueous TFA for example. When PG is a methoxymethylamine group or an ethoxymethylamine group the deprotection can be performed in the presence of aqueous HCl for example. When PG is an adamantyl carbamate group the deprotection can be realized by a treatment with by aqueous NaOH for example.

The deprotection step can be followed by the treatment of obtained compound 1S with a suitable boronic reagent R6B(OR′)₂, wherein —B(OR′)₂ is a boronic acid or a pinacolate ester and R6 is as defined above in STEP 11 using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent. When R6 is a substituted partially unsaturated cycloalkyl group, a partially unsaturated heterocycloalkyl group or non cyclic alkenyl group, compound I′ may be reduced by hydrogenation with a catalyst such as Pd/C under hydrogen pressure around 5 bars for example at temperature up to 70° C. to give the corresponding saturated compound I′.

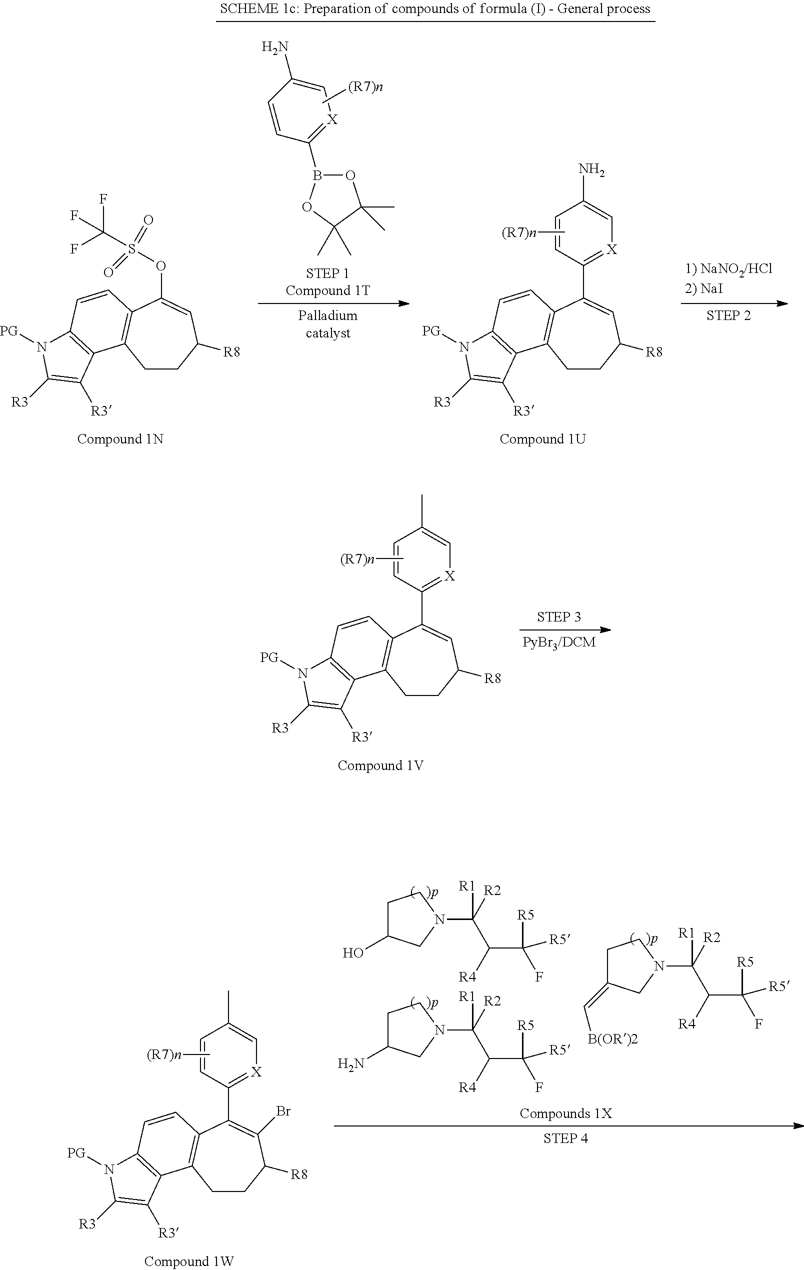

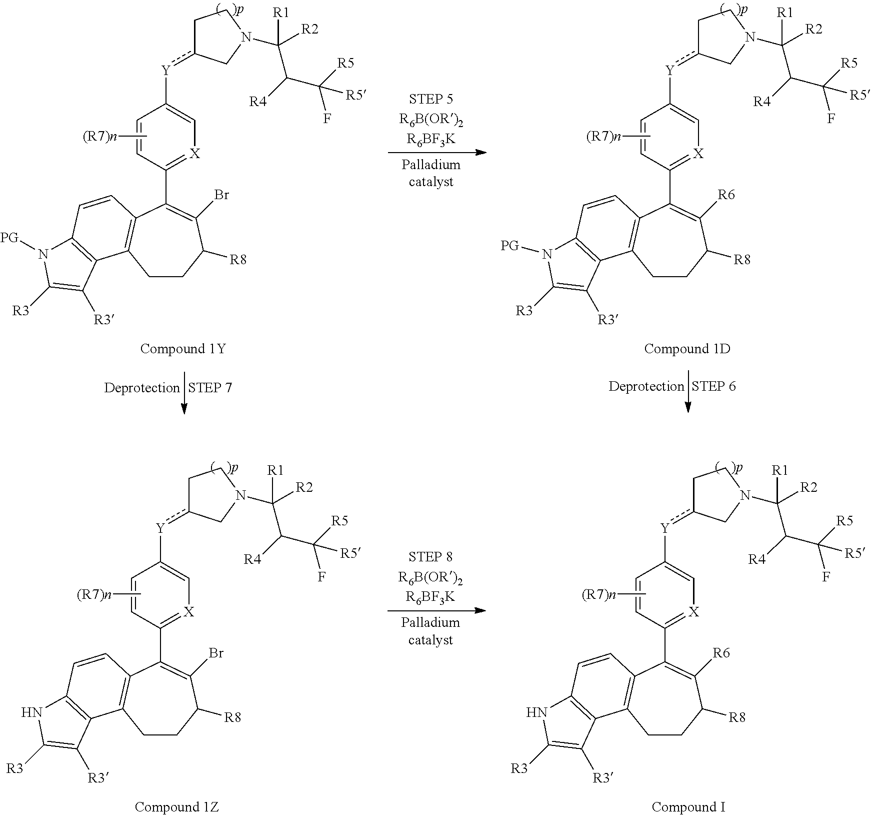

According to SCHEME 1c, in which R1, R2, R3, R3′, R4, R5, R5′, R6, R7, R8, n, p, X, [symbol] and Y are as defined above and PG is a protecting group (PG) such as a tosyl group, a benzenesulfonamide group, a t-butyl carbamate group, a methoxymethylamine group, a ethoxymethylamine group or a 1-adamantyl carbamate group, compound 1N can be converted in STEP 1 to compound 1U in a Suzuki coupling reaction with compound 1T using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1U can be converted in STEP 2 to compound 1V by treatment with sodium nitrite followed by a treatment with sodium iodide in solvents such as a mixture of water and acetonitrile.

Compound 1V can be converted in STEP 3 to compound 1W by treatment for example with pyridinium tribromide in DCM or THF at room temperature.

Compound 1W can be converted in STEP 4 to compound 1Y by a coupling reaction with one of compounds 1X in a coupling reaction conditions.

Compound 1Y can be converted to compound 1D in STEP 5 in a Suzuki coupling reaction with a suitable boronic reagent R6B(OR′)₂, wherein —B(OR′)₂ is a boronic acid or a pinacolate ester and R6 is as defined above using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

PG of compound 1D can be deprotected into compound I in STEP 6 using methods known in the litterature (Protective Groups in Organic Synthesis, Theodora W. Greene, Peter G. M. Wuts, John Wiley & Sons Inc). In particular when PG is a tosyl group or a benzenesulfonamide group the deprotection can be done by a treatment with an aqueous solution of potassium hydroxide in methanol for example. When PG is a t-butyl carbamate group, the deprotection can be performed in the presence of aqueous TFA for example. When PG is a methoxymethylamine group or an ethoxymethylamine group the deprotection can be performed in the presence of aqueous HCl for example. When PG is an adamantyl carbamate group the deprotection can be realized by a treatment with by aqueous NaOH for example.

Alternatively, PG of compound 1Y can be deprotected into compound 1Z in STEP 7 using methods known in the litterature (Protective Groups in Organic Synthesis, Theodora W. Greene, Peter G. M. Wuts, John Wiley & Sons Inc). In particular when PG is a tosyl group or a benzenesulfonamide group the deprotection can be done by a treatment with an aqueous solution of potassium hydroxide in methanol for example. When PG is a t-butyl carbamate group, the deprotection can be performed in the presence of aqueous TFA for example. When PG is a methoxymethylamine group or an ethoxymethylamine group the deprotection can be performed in the presence of aqueous HCl for example. When PG is an adamantyl carbamate group the deprotection can be realized by a treatment with by aqueous NaOH for example.

The deprotection step can be followed by the treatment of obtained compound 1Z with a suitable boronic reagent R6B(OR′)₂, wherein —B(OR′)₂ is a boronic acid or a pinacolate ester and R6 is as defined above in STEP 8 using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

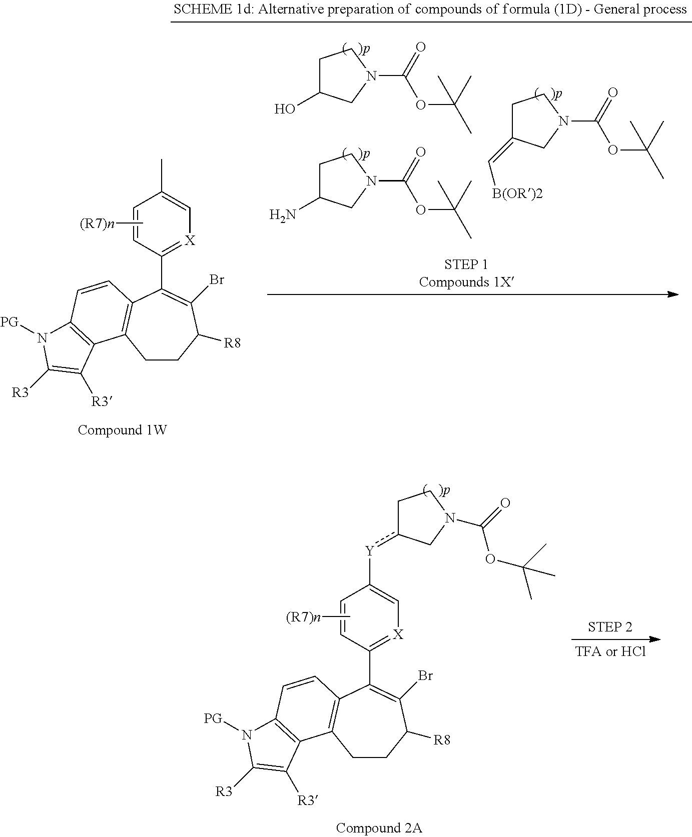

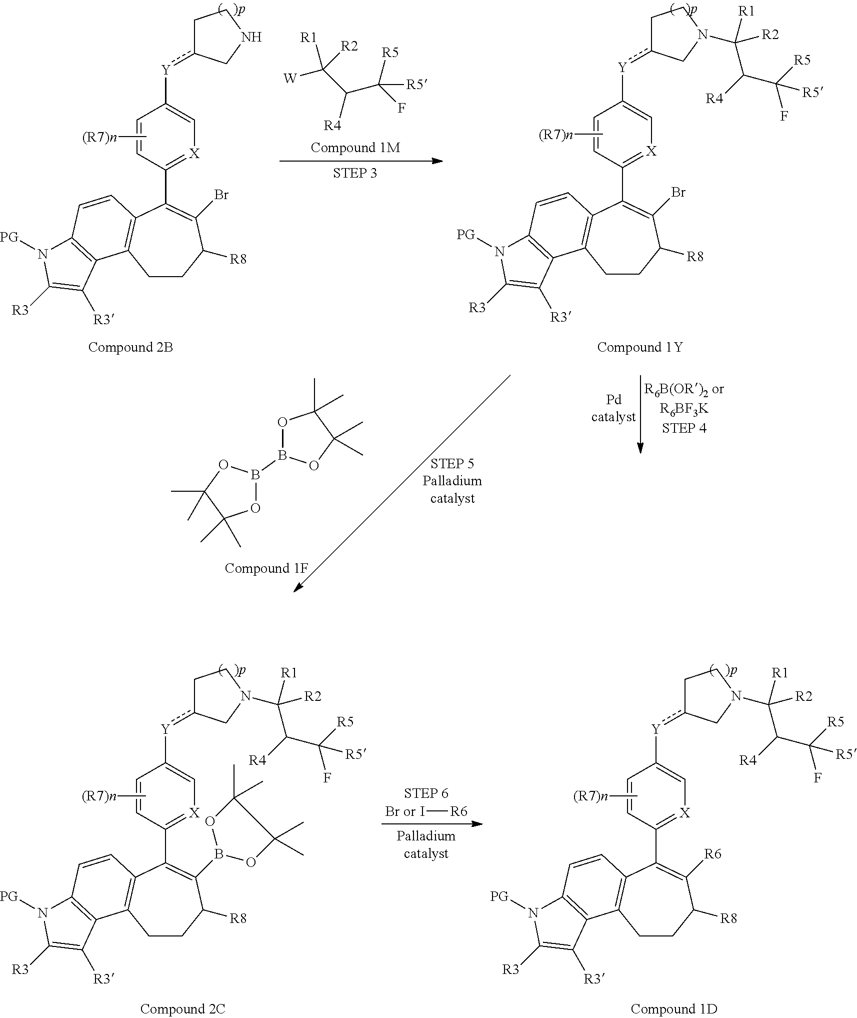

According to SCHEME 1d, in which R1, R2, R3, R3′, R4, R5, R5′, R6, R7, R8, n, p, X, [symbol] and Y are as defined above and PG is a protecting group (PG) such as a tosyl group, a benzenesulfonamide group, a para-methoxybenzylamine group, a 3,4-dimethoxybenzylamine group, a methoxymethylamine group, a ethoxymethylamine group or a 1-adamantyl carbamate group, compound 1W can be converted in STEP 1 to compound 2A by a coupling reaction with one of compounds 1X′ in a coupling reaction conditions.

Compound 2A, can be converted in STEP 2 to compound 2B by treatment with TFA or HCl.

Compound 2B can be converted in STEP 3 to compound 1Y by the treatment with compound 1M, wherein W is Cl, Br or I or OSO₂R with R═CH₃, PhMe, CF₃ or CF₂CF₂CF₂CF₃ in the presence of a base such as potassium carbonate (K₂CO₃) in DMF as a solvent.

Compound 1Y can be converted in STEP 5 to compound 2C by treatment for example with compound 1F, and with a palladium catalyst, for example bis (triphenylphosphine) palladium(II) dichloride Pd(PPh₃)₂Cl₂, and a phosphine, such as triphenylphosphine, in solution in toluene by heating up to reflux of solvent, in presence of a base such as KOPh.

Compound 1D can be prepared in a Suzuki coupling reaction either between compounds 1Y and with a suitable boronic reagent R6B(OR′)₂, wherein —B(OR′)₂ is a boronic acid or a pinacolate ester and R6 is as defined above in STEP 4 or between compounds 2C and either R6Br or R6I or R6OTf and R6 is as defined above in STEP 6 using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

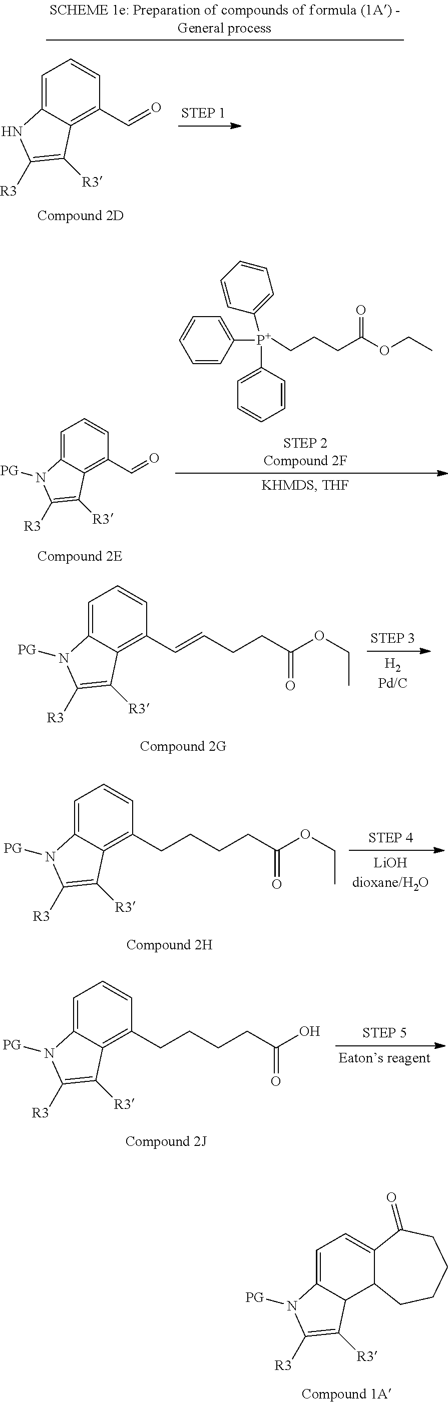

According to SCHEME 1e, in which R3 and R3′ are as defined above and PG is a tosyl group, compound 2D can be converted in STEP 1 to compound 2E by treatment with tosyl chloride in the presence of a base such as NaOH.

Compound 2E can be converted in STEP 2 to compound 2G by treatment with compound 2F in a Wittig reaction in the presence of a base such as KHMDS.

Compound 2H can be prepared by hydrogenation of compound 2G in STEP 3 with a catalyst, such as Pd/C under hydrogen (H₂) pressure.

Compound 2H can be converted in STEP 4 to compound 2J by treatment with a base such as LiOH.

Compound 1A′ can be prepared by treatment of compound 2J with Eaton's reagent.

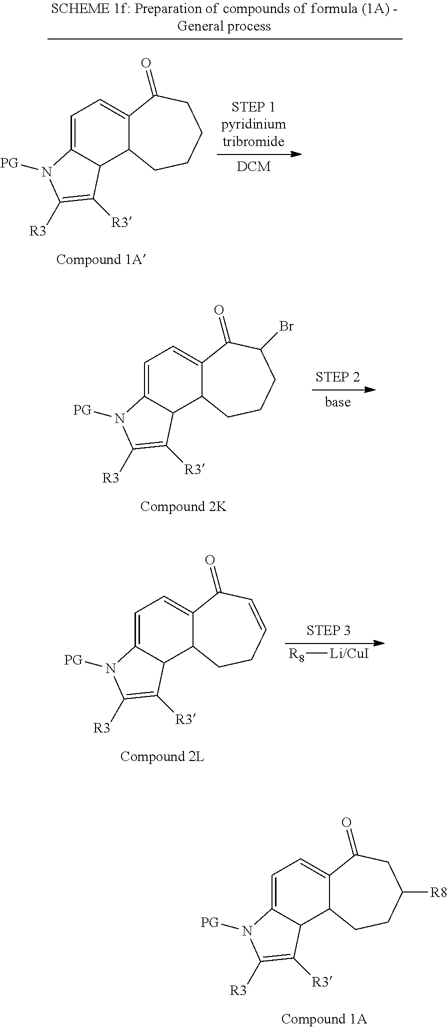

According to SCHEME 1f, in which R3, R3′ and R8 are as defined above and PG is a protecting group (PG) such as a tosyl group, compound 1A′ can be converted in STEP 1 to compound 2K by treatment with pyridinium tribromide.

Compound 2K can be converted in STEP 2 to compound 2L by treatment with a base such as DBU.

Compound 2L can be converted in STEP 3 to compound 1A by treatment with R8-Li in the presence of CuI in a solvent such as THF.

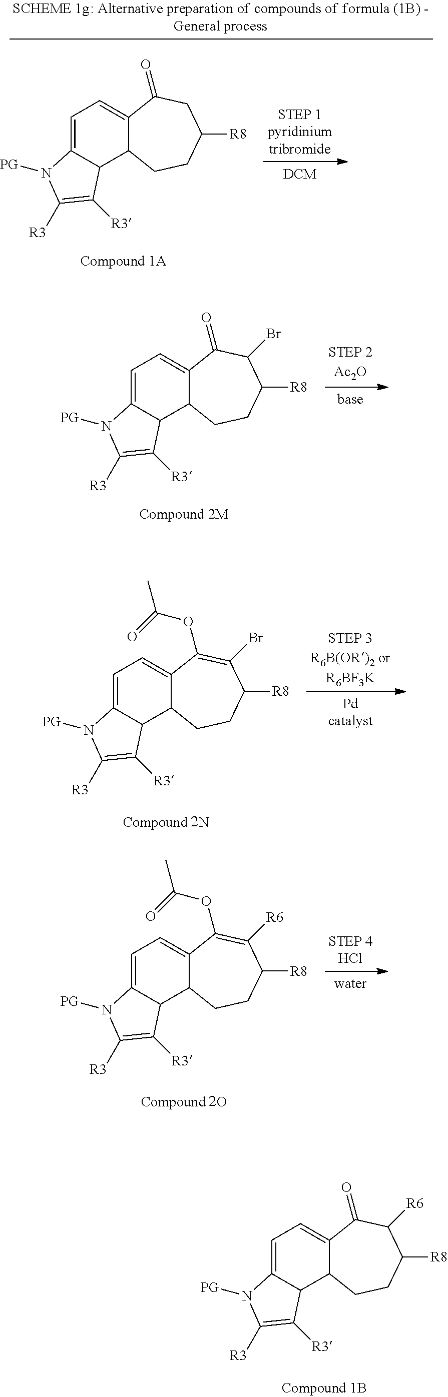

According to SCHEME 1g, in which R3, R3′, R6, and R8 are as defined above, and PG is a protecting group (PG) such as a tosyl group, compound 1B can alternatively be prepared as follows: compound 1A can be converted in STEP 1 to compound 2M by treatment with pyridinium tribromide in DCM or THF at room temperature for example.

Compound 2M can be converted in STEP 2 to compound 2N by deprotonation with a base such as LiHMDS in THF followed by treatment with acetic anhydride.

Compound 2O can be prepared in STEP 3 in a Suzuki coupling reaction between compounds 2N and R6B(OR′)₂, wherein —B(OR′)₂ is a boronic acid or a pinacolate ester, or R6BF₃K and R6 is as defined above using for example [1,1′-bis(diphenylphosphino) ferrocene]dichloropalladium(II) (Pd(dppf)Cl₂), complex with DCM, as catalyst, in a mixture of toluene and water and in the presence of a base, for example cesium carbonate (Cs₂CO₃), by heating up to reflux of solvent.

Compound 2O can be converted in STEP 4 to compound 1B by hydrolysis with aqueous HCl solution by heating in methanol and DCM for example.

Herein is also provided a process for preparing a compound of formula (I) as defined above, wherein a compound of formula 1D

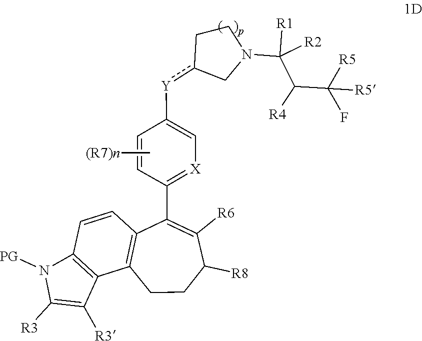

wherein R1, R2, R3, R3′, R4, R5, R5′, R6, R7, R8, n, p, X, [symbol] and Y are as defined above and PG is a protecting group such as a tosyl group, a benzenesulfonamide group, a t-butyl carbamate group, a methoxymethylamine, a ethoxymethylamine or a 1-adamantyl carbamate group, is converted to a compound of formula (I), by a deprotection step, said deprotection step being optionally followed by a hydrogenation step with a catalyst, such as Pd/C or platinum oxide (PtO₂) under hydrogen (H₂) pressure to give the corresponding compound of formula (I) wherein [symbol] is a single bond,
said deprotection step being optionally preceded by a step of obtaining compound 1D, wherein either a compound of formula 1C

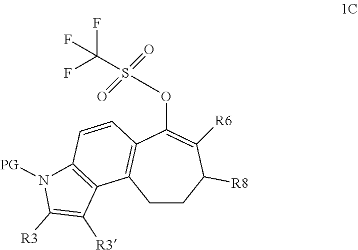

wherein R3, R3′, R6 and R8 are as defined above and PG is a protecting group such as defined above, or a compound 1G

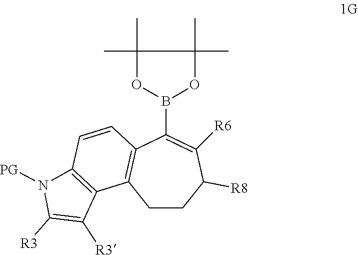

wherein R3, R3′, R6 and R8 are as defined above and PG is as defined above, is reacted in a Suzuki coupling step respectively with a compound 1E

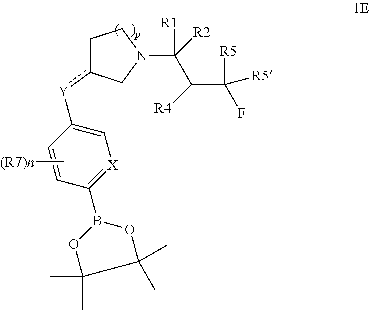

wherein R1, R2, R4, R5, R5′, R7, n, p, X, [symbol] and Y are as defined above
or with a compound 1H

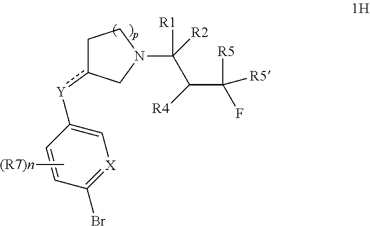

wherein R1, R2, R4, R5, R5′, R7, n, p, X, [symbol] and Y are as defined above, using a catalyst, for example [1,1′-bis(diphenylphosphino) ferrocene]dichloropalladium(II) (Pd(dppf)Cl₂) complex with DCM, in a solvent, for example in a mixture of dioxane and water and in the presence of a base, for example cesium carbonate (Cs₂CO₃), by heating up to reflux of solvent.

Herein is also provided a process for preparing a compound of formula (I) as defined above, wherein [symbol] is a single bond, wherein alternatively a compound of formula 1D′

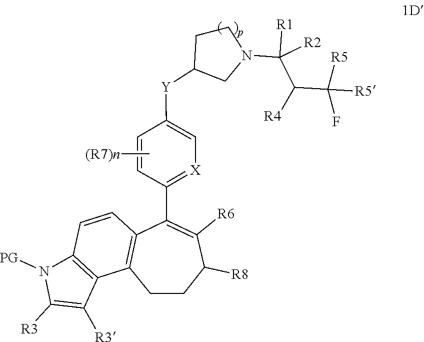

wherein R1, R2, R3, R3′, R4, R5, R5′, R6, R7, R8, n, p, X and Y are as defined above, and PG is a protecting group (PG) such as a tosyl group, a benzenesulfonamide group, a t-butyl carbamate group, a methoxymethylamine group, a ethoxymethylamine group or a 1-adamantyl carbamate group, is deprotected,
or a compound of formula 1S

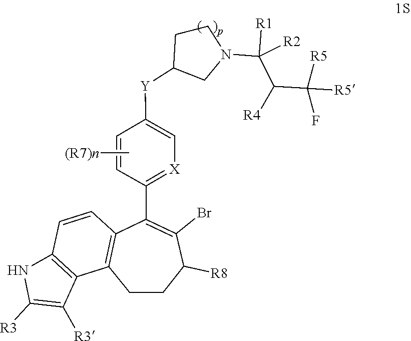

wherein R1, R2, R3, R3′, R4, R5, R5′, R7, R8, n, p, X and Y are as defined above, is reacted with a catalyst, such as [1,1′-bis(diphenylphosphino) ferrocene]dichloropalladium(II) (Pd(dppf)Cl₂) complex with DCM, in a solvent, such as a mixture of dioxane and water and in the presence of a base, for example cesium carbonate, by heating up to reflux of solvent,
said alternative steps being optionally preceded by a step of obtaining respectively a compound of formula 1D′ or a compound of formula 1S by respectively reacting a compound of formula 1Q

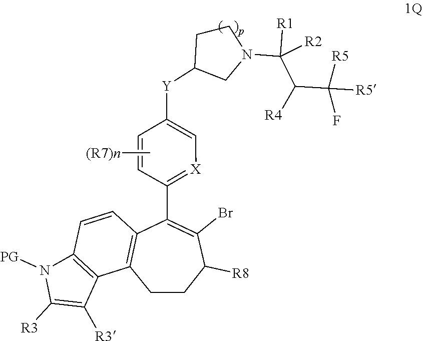

wherein R1, R2, R3, R3′, R4, R5, R5′, R6, R7, R8, n, p, X and Y are as defined above, and PG is a protecting group such as defined above,
with a boronic reagent R6B(OR′)₂, wherein —B(OR′)₂ is a boronic acid or a pinacolate ester and R6 is as defined above for obtaining said compound of formula 1D′,
or
by a deprotection step, in particular by treatment with an aqueous solution of potassium hydroxide in methanol, for obtaining said compound of formula 1S.

Herein are also provided the intermediate compounds selected from compounds of formula 1D, 1K, 1L, 1D′, 1O, 1Q, 1S, 1R, 1Y, 1Z, 2A and 2C, or any of its pharmaceutically acceptable salt,

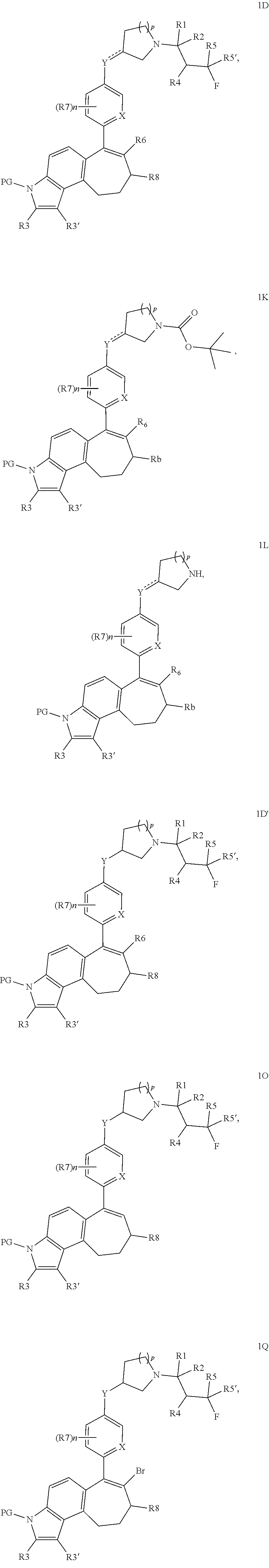

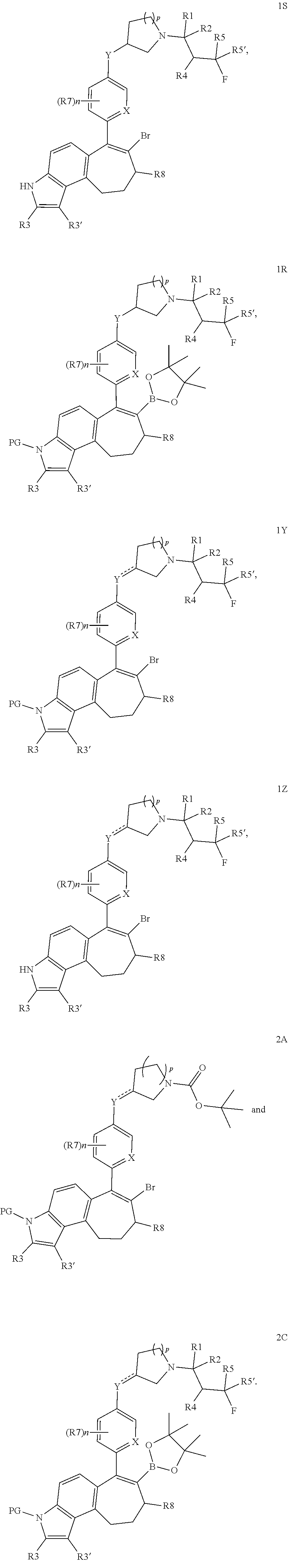

wherein R1, R2, R3, R3′, R4, R5, R5′, R6, R7, R8, n, p, X, [symbol] and Y are as defined above and PG is a protecting group such as defined above, such as a tosyl group.

The ¹H NMR Spectra at 400 and 500 MHz were performed on a Bruker Avance DRX-400 and Bruker Avance DPX-500 spectrometer, respectively, with the chemical shifts (δ in ppm) in the solvent dimethyl sulfoxide-d6 (d6-DMSO) referenced at 2.5 ppm at a temperature of 303 K. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210-400 nm and flash Acquity UPLC CSH C18 1.7 μm, dimension 2.1×30 mm, mobile phase H₂O+0.1% HCO₂H/CH₃CN+0.1% HC₂H.

The following tables 1a and 1b comprises respectively specific compounds of formula (I) (name and structure) in accordance with the present disclosure as well their characterization (H NMR and liquid chromatography/mass).

TABLE 1a

| Example | | |
|---|---|---|
| or | | |
| compound | Structure | Name |
| 1 | 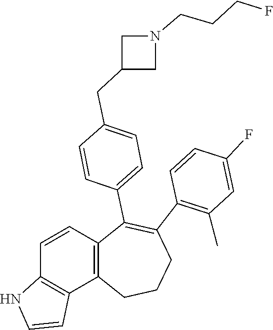 [SMILES: Cc1cc(F)ccc1C1=C(c2ccc(CC3CN(CCCF)C3)cc2)c2ccc3[nH]ccc3c2CCC1] | 7-(4-fluoro-2-methylphenyl)-6-(4-((1- (3-fluoropropyl)azetidin-3- yl)methyl)phenyl)-3,8,9,10- tetrahydrocyclohepta[e]indole |
| 2 | 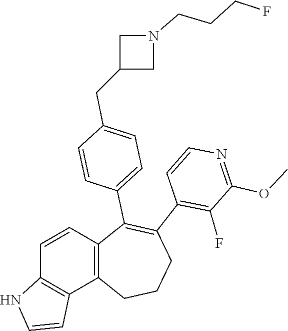 [SMILES: COc1nccc(C2=C(c3ccc(CC4CN(CCCF)C4)cc3)c3ccc4[nH]ccc4c3CCC2)c1F] | 7-(3-fluoro-2-methoxypyridin-4-yl)-6- (4-((1-(3-fluoropropyl)azetidin-3- yl)methyl)phenyl)-3,8,9,10- tetrahydrocyclohepta[e]indole |
| 3 | 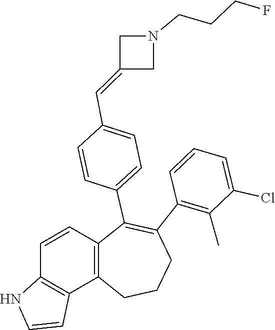 [SMILES: Cc1c(Cl)cccc1C1=C(c2ccc(C=C3CN(CCCF)C3)cc2)c2ccc3[nH]ccc3c2CCC1] | 7-(3-chloro-2-methylphenyl)-6-(4-((1- (3-fluoropropyl)azetidin-3- ylidene)methyl)phenyl)-3,8,9,10- tetrahydrocyclohepta[e]indole |
| 4 | 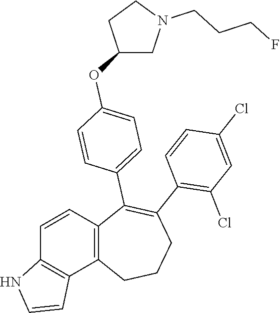 [SMILES: FCCCN1CC[C@H](Oc2ccc(C3=C(c4ccc(Cl)cc4Cl)CCCc4c3ccc3[nH]ccc43)cc2)C1] | (S)-7-(2,4-dichlorophenyl)-6-(4-((1-(3- fluoropropyl)pyrrolidin-3- yl)oxy)phenyl)-3,8,9,10- tetrahydrocyclohepta[e]indole |
| 5 | 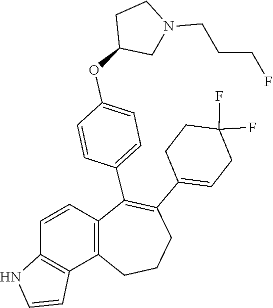 [SMILES: FCCCN1CC[C@H](Oc2ccc(C3=C(C4=CCC(F)(F)CC4)CCCc4c3ccc3[nH]ccc43)cc2)C1] | (S)-7-(4,4-difluorocyclohex-1-en-1-yl)- 6-(4-((1-(3-fluoropropyl)pyrrolidin-3- yl)oxy)phenyl)-3,8,9,10- tetrahydrocyclohepta[e]indole |
| 6 | 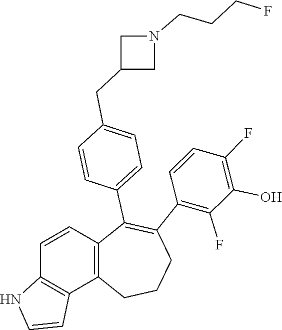 [SMILES: Oc1c(F)ccc(C2=C(c3ccc(CC4CN(CCCF)C4)cc3)c3ccc4[nH]ccc4c3CCC2)c1F] | 2,6-difluoro-3-(6-(4-((1-(3- fluoropropyl)azetidin-3- yl)methyl)phenyl)-3,8,9,10- tetrahydrocyclohepta[e]indol-7- yl)phenol |
| 7 | 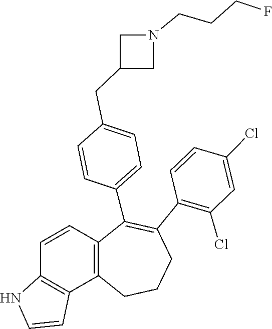 [SMILES: FCCCN1CC(Cc2ccc(C3=C(c4ccc(Cl)cc4Cl)CCCc4c3ccc3[nH]ccc43)cc2)C1] | 7-(2,4-dichlorophenyl)-6-(4-((1-(3- fluoropropyl)azetidin-3- yl)methyl)phenyl)-3,8,9,10- tetrahydrocyclohepta[e]indole |
| 8 | 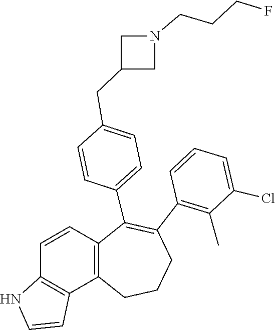 [SMILES: Cc1c(Cl)cccc1C1=C(c2ccc(CC3CN(CCCF)C3)cc2)c2ccc3[nH]ccc3c2CCC1] | 7-(3-chloro-2-methylphenyl)-6-(4-((1- (3-fluoropropyl)azetidin-3- yl)methyl)phenyl)-3,8,9,10- tetrahydrocyclohepta[e]indole |
| 9 | 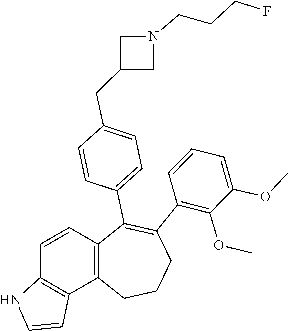 [SMILES: COc1cccc(C2=C(c3ccc(CC4CN(CCCF)C4)cc3)c3ccc4[nH]ccc4c3CCC2)c1OC] | 7-(2,3-dimethoxyphenyl)-6-(4-((1-(3- fluoropropyl)azetidin-3- yl)methyl)phenyl)-3,8,9,10- tetrahydrocyclohepta[e]indole |
| 10 | 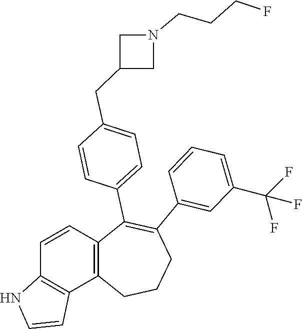 [SMILES: FCCCN1CC(Cc2ccc(C3=C(c4cccc(C(F)(F)F)c4)CCCc4c3ccc3[nH]ccc43)cc2)C1] | 6-(4-((1-(3-fluoropropyl)azetidin-3- yl)methyl)phenyl)-7-(3- (trifluoromethyl)phenyl)-3,8,9,10- tetrahydrocyclohepta[e]indole |
| 11 | 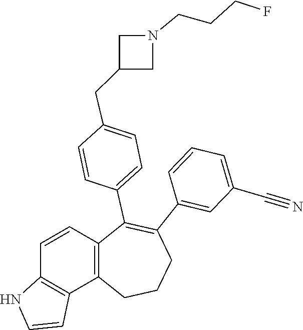 [SMILES: N#Cc1cccc(C2=C(c3ccc(CC4CN(CCCF)C4)cc3)c3ccc4[nH]ccc4c3CCC2)c1] | 3-(6-(4-((1-(3-fluoropropyl)azetidin-3- yl)methyl)phenyl)-3,8,9,10- tetrahydrocyclohepta[e]indol-7- yl)benzonitrile |
| 12 | 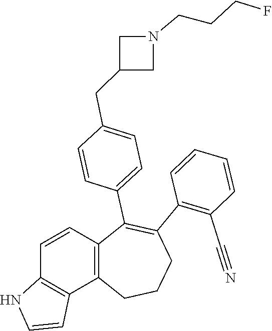 [SMILES: N#Cc1ccccc1C1=C(c2ccc(CC3CN(CCCF)C3)cc2)c2ccc3[nH]ccc3c2CCC1] | 2-(6-(4-((1-(3-fluoropropyl)azetidin-3- yl)methyl)phenyl)-3,8,9,10- tetrahydrocyclohepta[e]indol-7- yl)benzonitrile |
| 13 | 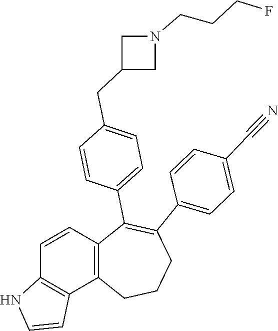 [SMILES: N#Cc1ccc(C2=C(c3ccc(CC4CN(CCCF)C4)cc3)c3ccc4[nH]ccc4c3CCC2)cc1] | 4-(6-(4-((1-(3-fluoropropyl)azetidin-3- yl)methyl)phenyl)-3,8,9,10- tetrahydrocyclohepta[e]indol-7- yl)benzonitrile |
| 14 | 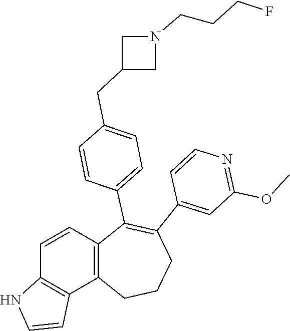 [SMILES: COc1cc(C2=C(c3ccc(CC4CN(CCCF)C4)cc3)c3ccc4[nH]ccc4c3CCC2)ccn1] | 6-(4-((1-(3-fluoropropyl)azetidin-3- yl)methyl)phenyl)-7-(2- methoxypyridin-4-yl)-3,8,9,10- tetrahydrocyclohepta[e]indole |
| 15 | 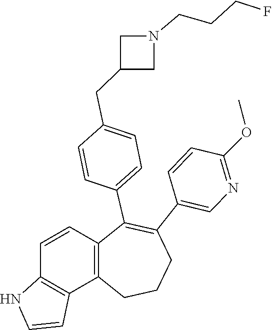 [SMILES: COc1ccc(C2=C(c3ccc(CC4CN(CCCF)C4)cc3)c3ccc4[nH]ccc4c3CCC2)cn1] | 6-(4-((1-(3-fluoropropyl)azetidin-3- yl)methyl)phenyl)-7-(6- methoxypyridin-3-yl)-3,8,9,10- tetrahydrocyclohepta[e]indole |
| 16 | 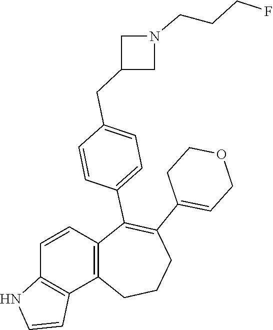 [SMILES: FCCCN1CC(Cc2ccc(C3=C(C4=CCOCC4)CCCc4c3ccc3[nH]ccc43)cc2)C1] | 7-(3,6-dihydro-2H-pyran-4-yl)-6-(4- ((1-(3-fluoropropyl)azetidin-3- yl)methyl)phenyl)-3,8,9,10- tetrahydrocyclohepta[e]indole |
| 17 | 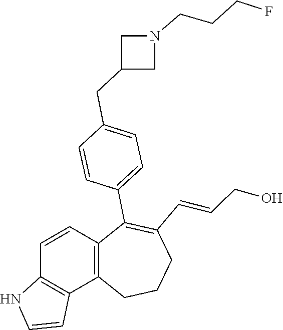 [SMILES: OC/C=C/C1=C(c2ccc(CC3CN(CCCF)C3)cc2)c2ccc3[nH]ccc3c2CCC1] | (E)-3-(6-(4-((1-(3- fluoropropyl)azetidin-3- yl)methyl)phenyl)-3,8,9,10- tetrahydrocyclohepta[e]indol-7- yl)prop-2-en-1-ol |
| 18 | 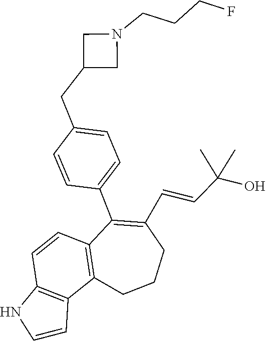 [SMILES: CC(C)(O)/C=C/C1=C(c2ccc(CC3CN(CCCF)C3)cc2)c2ccc3[nH]ccc3c2CCC1] | (E)-4-(6-(4-((1-(3- fluoropropyl)azetidin-3- yl)methyl)phenyl)-3,8,9,10- tetrahydrocyclohepta[e]indol-7-yl)-2- methylbut-3-en-2-ol |
| 19 | 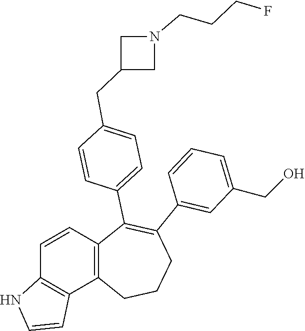 [SMILES: OCc1cccc(C2=C(c3ccc(CC4CN(CCCF)C4)cc3)c3ccc4[nH]ccc4c3CCC2)c1] | (3-(6-(4-((1-(3-fluoropropyl)azetidin-3- yl)methyl)phenyl)-3,8,9,10- tetrahydrocyclohepta[e]indol-7- yl)phenyl)methanol |
| 20 | 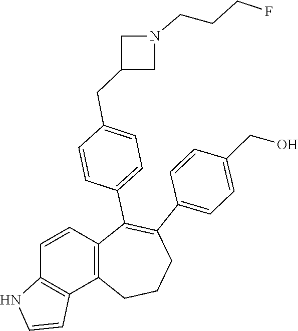 [SMILES: OCc1ccc(C2=C(c3ccc(CC4CN(CCCF)C4)cc3)c3ccc4[nH]ccc4c3CCC2)cc1] | (4-(6-(4-((1-(3-fluoropropyl)azetidin-3- yl)methyl)phenyl)-3,8,9,10- tetrahydrocyclohepta[e]indol-7- yl)phenyl)methanol |
| 21 | 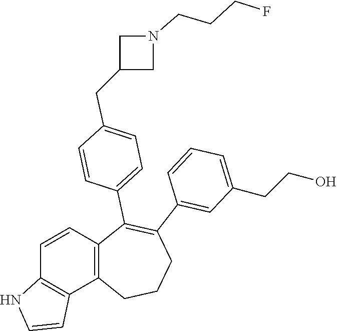 [SMILES: OCCc1cccc(C2=C(c3ccc(CC4CN(CCCF)C4)cc3)c3ccc4[nH]ccc4c3CCC2)c1] | 2-(3-(6-(4-((1-(3-fluoropropyl)azetidin- 3-yl)methyl)phenyl)-3,8,9,10- tetrahydrocyclohepta[e]indol-7- yl)phenyl)ethan-1-ol |
| 22 | 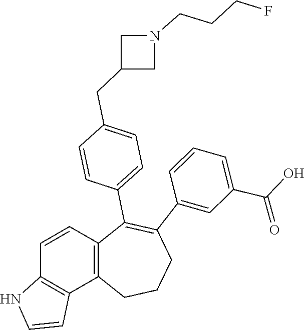 [SMILES: O=C(O)c1cccc(C2=C(c3ccc(CC4CN(CCCF)C4)cc3)c3ccc4[nH]ccc4c3CCC2)c1] | 3-(6-(4-((1-(3-fluoropropyl)azetidin-3- yl)methyl)phenyl)-3,8,9,10- tetrahydrocyclohepta[e]indol-7- yl)benzoic acid |
| 23 | 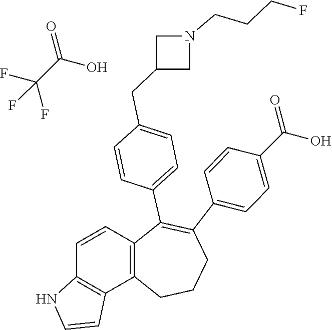 [SMILES: O=C(O)C(F)(F)F ; O=C(O)c1ccc(C2=C(c3ccc(CC4CN(CCCF)C4)cc3)c3ccc4[nH]ccc4c3CCC2)cc1] | 4-(6-(4-((1-(3-fluoropropyl)azetidin-3- yl)methyl)phenyl)-3,8,9,10- tetrahydrocyclohepta[e]indol-7- yl)benzoic acid, 2,2,2-trifluoroacetic acid |
| 24 | 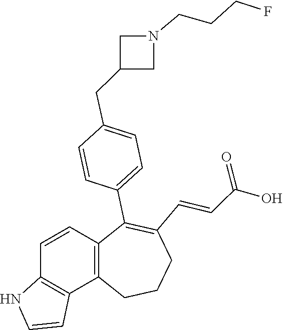 [SMILES: O=C(O)/C=C/C1=C(c2ccc(CC3CN(CCCF)C3)cc2)c2ccc3[nH]ccc3c2CCC1] | (E)-3-(6-(4-((1-(3- fluoropropyl)azetidin-3- yl)methyl)phenyl)-3,8,9,10- tetrahydrocyclohepta[e]indol-7- yl)acrylic acid |
| 25 | 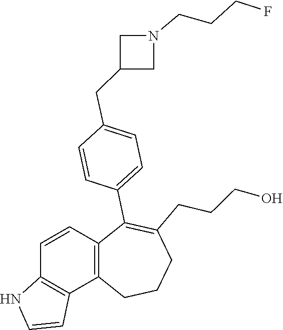 [SMILES: OCCCC1=C(c2ccc(CC3CN(CCCF)C3)cc2)c2ccc3[nH]ccc3c2CCC1] | 3-(6-(4-((1-(3-fluoropropyl)azetidin-3- yl)methyl)phenyl)-3,8,9,10- tetrahydrocyclohepta[e]indol-7- yl)propan-1-ol |
| 26 | 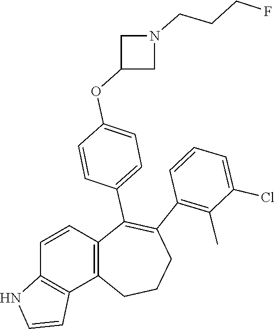 [SMILES: Cc1c(Cl)cccc1C1=C(c2ccc(OC3CN(CCCF)C3)cc2)c2ccc3[nH]ccc3c2CCC1] | 7-(3-chloro-2-methylphenyl)-6-(4-((1- (3-fluoropropyl)azetidin-3- yl)oxy)phenyl)-3,8,9,10- tetrahydrocyclohepta[e]indole |
| 27 | 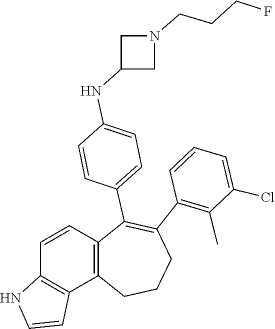 [SMILES: Cc1c(Cl)cccc1C1=C(c2ccc(NC3CN(CCCF)C3)cc2)c2ccc3[nH]ccc3c2CCC1] | N-(4-(7-(3-chloro-2-methylphenyl)- 3,8,9,10-tetrahydrocyclohepta[e]indol- 6-yl)phenyl)-1-(3- fluoropropyl)azetidin-3-amine |
| 28 | 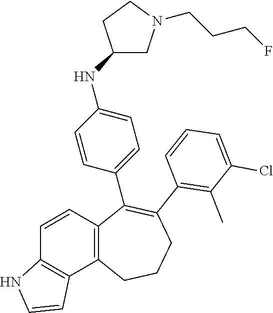 [SMILES: Cc1c(Cl)cccc1C1=C(c2ccc(N[C@H]3CCN(CCCF)C3)cc2)c2ccc3[nH]ccc3c2CCC1] | (S)-N-(4-(7-(3-chloro-2- methylphenyl)-3,8,9,10- tetrahydrocyclohepta[e]indol-6- yl)phenyl)-1-(3- fluoropropyl)pyrrolidin-3-amine |
| 29 | 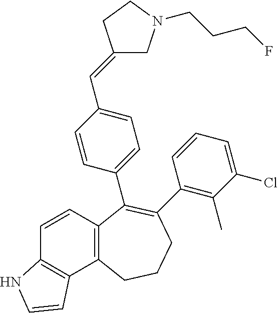 [SMILES: Cc1c(Cl)cccc1C1=C(c2ccc(/C=C3/CCN(CCCF)C3)cc2)c2ccc3[nH]ccc3c2CCC1] | (Z)-7-(3-chloro-2-methylphenyl)-6-(4- ((1-(3-fluoropropyl)pyrrolidin-3- ylidene)methyl)phenyl)-3,8,9,10- tetrahydrocyclohepta[e]indole |

TABLE 1b

| | | | MASS: |
|---|---|---|---|
| Example | | | LC/MS |
| or | Preparation | | (m/z, |
| compound | Method | NMR | MH+): |
| 1 | A | 1H NMR (400 MHz, DMSO-d6) δ ppm 1.50-1.68 | 497 |
| | | (m, 2 H), 2.12-2.26 (m, 4 H), 2.19 (s, 3 H), | |
| | | 2.38 (t, J = 7 Hz, 2 H), 2.45-2.60 (m partially | |
| | | hidden, 1 H), 2.64-2.73 (m, 4 H), 3.07-3.14 | |
| | | (m, 2 H), 3.21 (br t, J = 7 Hz, 2 H), 4.42 (dt, J = 48, | |
| | | 6 Hz, 2 H), 6.49 (d, J = 9 Hz, 1 H), 6.60 (m, 1 H), | |
| | | 6.70 (d, J = 8 Hz, 2 H), 6.79-6.89 (m, 1 H), 6.85 | |
| | | (d, J = 8 Hz, 2 H), 6.95 (dd, J = 10, 3 Hz, 1 H), 7.06 | |
| | | (dd, J = 8, 6 Hz, 1 H), 7.16 (d, J = 8 Hz, 1 H), 7.34 | |
| | | (t, J = 3 Hz, 1 H), 11.06 (br s, 1 H) | |
| 2 | B | 1H NMR (400 MHz, DMSO-d6) δ ppm 1.51-1.698 | 514 |
| | | (m, 2 H), 2.13-2.25 (m, 4 H), 2.37 (t, J = 7 | |
| | | Hz, 2 H), 2.45-2.60 (m partially hidden, 1 H), | |
| | | 2.63-2.73 (m, 4 H), 3,00-3.10 (m, 2 H), 3.20 (t, | |
| | | J = 7 Hz, 2 H), 3.86 (s, 3 H), 4.42 (dt, J = 48, 6 Hz, | |
| | | 2 H), 6.47 (d, J = 9 Hz, 1 H), 6.59-6.62 (m, 1 H), | |
| | | 6.74-6.83 (m, 3 H), 6.92 (d, J = 8 Hz, 2 H), 7.17 | |
| | | (d, J = 9 Hz, 1 H), 7.36 (t, J = 3 Hz, 1 H), 7.75 (d, | |
| | | J = 5 Hz, 1 H), 11.13 (br s, 1 H) | |
| 3 | C | 1H NMR (400 MHz, DMSO-d6) δ ppm 1.60-1.80 | 511 |
| | | (m, 2 H) 2.07-2.30 (m, 4 H) 2.27 (s, 3H) | |
| | | 2.69-2.88 (m, 2 H) 3.02-3.20 (m, 2 H) 3.92-4.40 | |
| | | (m, 4 H) 4.47 (dt, J = 47, 6 Hz, 2 H) 6.07-6.16 | |
| | | (m, 1 H) 6.48 (d, J = 8 Hz, 1 H) 6.61 (br s, 1 | |
| | | H) 6.75 (d, J = 8 Hz, 2 H) 6.85 (br d, J = 8 Hz, 2 H) | |
| | | 6.97-7.10 (m, 2 H) 7.16 (d, J = 8 Hz, 1 H) 7.23 | |
| | | (dd, J = 7, 2 Hz, 1 H) 7.36 (t, J = 3 Hz, 1 H) | |
| | | 9.90-10.3 (m, 1H) 11.07-11.13 (m, 1 H) | |
| 4 | A | 1H NMR (500 MHz, DMSO-d6) δ ppm 1.62-1.73 | 549 |
| | | (m, 1 H), 1.73-1.85 (m, 2 H), 2.19 (br s, 5 | |
| | | H), 2.34-2.58 (m partially hidden, 4 H), 2.60-2.71 | |
| | | (m partially hidden, 1 H), 2.79 (m, 1 H), | |
| | | 2.99-3.11 (m, 1 H), 3.14-3.22 (m, 1 H), 4.46 | |
| | | (dt, J = 48, 6 Hz, 2 H), 4.70-4.80 (m, 1 H), 6.50 | |
| | | (d, J = 8 Hz, 1 H), 6.58-6.60 (m, 1 H), 6.62 (d, | |
| | | J = 9 Hz, 2 H), 6.74 (d, J = 9 Hz, 2 H), 7.16 (dd, | |
| | | J = 8, 1 Hz, 1 H), 7.19 (d, J = 8 Hz, 1 H), 7.24 (dd, | |
| | | J = 8, 2 Hz, 1 H), 7.34 (t, J = 3 Hz, 1 H), 7.56 (d, | |
| | | J = 2 Hz, 1 H), 11.07 (br s, 1 H) | |
| 5 | A | 1H NMR (500 MHz, DMSO-d6) δ ppm 1.72-1.97 | 521 |
| | | (m, 5 H), 1.99-2.04 (m, 2 H), 2.13-2.29 | |
| | | (m, 5 H), 2.38-2.57 (m partially hidden, 5 H), | |
| | | 2.61 (dd partially hidden, J = 10, 3 Hz, 1 H), | |
| | | 2.65-2.73 (m, 1 H), 2.83 (dd, J = 10, 6 Hz, 1 H), 2.92 | |
| | | (t, J = 7 Hz, 2 H), 4.48 (dt, J = 48, 6 Hz, 2 H), | |
| | | 4.77-4.87 (m, 1 H), 5.34-5.41 (m, 1 H), 6.48 (d, J = 9 | |
| | | Hz, 1 H), 6.51-6.56 (m, 1 H), 6.74 (d, J = 9 Hz, 2 | |
| | | H), 6.95 (d, J = 9 Hz, 2 H), 7.12 (dd, J = 8, 1 Hz, 1 | |
| | | H), 7.30 (t, J = 3 Hz, 1 H), 11.00 (br s, 1 H) | |
| 6 | A | 1H NMR (400 MHz, DMSO-d6) δ ppm 1.52-1.69 | 517 |
| | | (m, 2 H), 2.19 (br s, 4 H), 2.38 (t, J = 7 Hz, 2 | |
| | | H), 2.48-2.61 (m partially hidden, 1 H), 2.66-2.72 | |
| | | (m, 4 H), 3.00-3.12 (m, 2 H), 3.22 (br t | |
| | | partially hidden, J = 7 Hz, 2 H), 4.42 (dt, J = 48, 6 | |
| | | Hz, 2 H), 6.48 (d, J = 8 Hz, 1 H), 6.51-6.64 (m, 2 | |
| | | H), 6.77 (d, J = 8 Hz, 2 H), 6.82 (br t, J = 10 Hz, 1 | |
| | | H), 6.89 (d, J = 8 Hz, 2 H), 7.16 (d, J = 9 Hz, 1 H), | |
| | | 7.34 (t, J = 3 Hz, 1 H), 11.08 (br s, 1 H) | |
| 7 | B | 1H NMR (400 MHz, DMSO-d6) δ ppm 1.50-1.68 | 533 |
| | | (m, 2 H), 2.14-2.28 (m, 4 H), 2.37 (br t, | |
| | | J = 7 Hz, 2 H), 2.41-2.60 (m partially hidden, 1 | |
| | | H), 2.62-2.72 (m, 4 H), 2.97-3.11 (m, 1 H), | |
| | | 3.15-3.25 (m, 3 H), 4.42 (dt, J = 48, 6 Hz, 2 H), | |
| | | 6.47 (d, J = 8 Hz, 1 H), 6.58-6.62 (m, 1 H), 6.76 | |
| | | (d, J = 8 Hz, 2 H), 6.89 (d, J = 8 Hz, 2 H), | |
| | | 7.13-7.19 (m, 2 H), 7.20-7.26 (m, 1 H), 7.35 (br t, | |
| | | J = 3 Hz, 1 H), 7.57 (br d, J = 2 Hz, 1 H), 11.11 (br | |
| | | s, 1 H) | |
| 8 | B | 1H NMR (400 MHz, DMSO-d6) δ ppm 1.51-1.69 | 513 |
| | | (m, 2 H), 2.09-2.27 (m, 4 H), 2.24 (s, 3 H), | |
| | | 2.37 (t, J = 7 Hz, 2 H), 2.46-2.59 (m partially | |
| | | hidden, 1 H), 2.62-2.72 (m, 2 H), 2.67 (br d, | |
| | | J = 7 Hz, 2 H), 3.06-3.16 (m, 2 H), 3.19 (t, J = 7 | |
| | | Hz, 2 H), 4.42 (dt, J = 48, 6 Hz, 2 H), 6.50 (d, J = 9 | |
| | | Hz, 1 H), 6.58-6.62 (m, 1 H), 6.70 (d, J = 8 Hz, 2 | |
| | | H), 6.86 (d, J = 8 Hz, 2 H), 6.98-7.08 (m, 2 H), | |
| | | 7.16 (d, J = 8 Hz, 1 H), 7.22 (dd, J = 7, 2 Hz, 1 H), | |
| | | 7.33-7.37 (m, 1 H), 11.07 (br s, 1 H) | |
| 9 | B | 1H NMR (400 MHz, DMSO-d6) δ ppm 1.50-1.68 | 525 |
| | | (m, 2 H), 2.07-2.23 (m, 4 H), 2.36 (t, J = 7 | |
| | | Hz, 2 H), 2.46-2.58 (m partially hidden, 1 H), | |
| | | 2.62-2.71 (m, 4 H), 2.97-3.15 (m, 2 H), 3.19 | |
| | | (br t, J = 7 Hz, 2 H), 3.68 (s, 3 H), 3.77 (s, 3 H), | |
| | | 4.41 (dt, J = 48, 6 Hz, 2 H), 6.47 (d, J = 8 Hz, 1 H), | |
| | | 6.54-6.61 (m, 2 H), 6.74-6.88 (m, 6 H), 7.14 | |
| | | (br d, J = 8 Hz, 1 H), 7.30-7.35 (m, 1 H), 11.06 | |
| | | (br s, 1 H) | |
| 10 | B | 1H NMR (400 MHz, DMSO-d6) δ ppm 1.50-1.65 | 533 |
| | | (m, 2 H), 2.14-2.26 (m, 2 H), 2.30-2.40 | |
| | | (m, 4 H), 2.43-2.58 (m partially hidden, 1 H), | |
| | | 2.64-2.72 (m, 4 H), 3.06 (br t, J = 7 Hz, 2 H), | |
| | | 3.14-3.21 (m, 2 H), 4.42 (dt, J = 48, 6 Hz, 2 H), | |
| | | 6.49 (d, J = 8 Hz, 1 H), 6.58-6.62 (m, 1 H), 6.75 | |
| | | (d, J = 8 Hz, 2 H), 6.90 (d, J = 8 Hz, 2 H), 7.15 (d, | |
| | | J = 8 Hz, 1 H), 7.32 (m, 1 H), 7.35 (t, J = 3 Hz, 1 | |
| | | H), 7.41-7.45 (m, 2 H), 7.49-7.55 (m, 1 H), | |
| | | 11.10 (br s, 1 H) | |
| 11 | B | 1H NMR (400 MHz, DMSO-d6) δ ppm 1.50-1.67 | 490 |
| | | (m, 2 H), 2.13-2.24 (m, 2 H), 2.26-2.34 | |
| | | (m, 2 H), 2.38 (t, J = 7 Hz, 2 H), 2.45-2.61 (m | |
| | | partially hidden, 1 H), 2.63-2.72 (m, 4 H), 3.06 | |
| | | (br t, J = 7 Hz, 2 H), 3.21 (t, J = 7 Hz, 2 H), 4.42 | |
| | | (dt, J = 48, 6 Hz, 2 H), 6.48 (d, J = 8 Hz, 1 H), | |
| | | 6.58-6.62 (m, 1 H), 6.76 (d, J = 8 Hz, 2 H), 6.92 (d, | |
| | | J = 8 Hz, 2 H), 7.15 (d, J = 8 Hz, 1 H), 7.35 (t, J = 3 | |
| | | Hz, 1 H), 7.35-7.41 (m, 1 H), 7.47-7.52 (m, 2 | |
| | | H), 7.55 (br d, J = 8 Hz, 1 H), 11.10 (br s, 1 H) | |
| 12 | B | 1H NMR (400 MHz, DMSO-d6) δ ppm 1.51-1.68 | 490 |
| | | (m, 2 H), 2.15-2.31 (m, 4 H), 2.37 (br t, | |
| | | J = 7 Hz, 2 H), 2.43-2.60 (m partially hidden, 1 | |
| | | H), 2.62-2.75 (m, 4 H), 3.15-3.26 (m, 4 H), | |
| | | 4.42 (dt, J = 48, 6 Hz, 2 H), 6.47 (d, J = 9 Hz, 1 H), | |
| | | 6.59-6.63 (m, 1 H), 6.74 (d, J = 8 Hz, 2 H), 6.86 | |
| | | (d, J = 8 Hz, 2 H), 7.17 (d, J = 8 Hz, 1 H), 7.30-7.40 | |
| | | (m, 2 H), 7.46 (d, J = 8 Hz, 1 H), 7.58 (br t, | |
| | | J = 8 Hz, 1 H), 7.64 (d, J = 8 Hz, 1 H), 11.13 (br s, | |
| | | 1 H) | |
| 13 | B | 1H NMR (400 MHz, DMSO-d6) δ ppm 1.51-1.68 | 490 |
| | | (m, 2 H), 2.12-2.23 (m, 2 H), 2.27-2.34 | |
| | | (m, 2 H), 2.38 (t, J = 7 Hz, 2 H), 2.44-2.61 (m | |
| | | partially hidden, 1 H), 2.65-2.73 (m, 4 H), 3.05 | |
| | | (br t, J = 7 Hz, 2 H), 3.22 (t, J = 7 Hz, 2 H), 4.42 | |
| | | (dt, J = 48, 6 Hz, 2 H), 6.47 (d, J = 8 Hz, 1 H), | |
| | | 6.59-6.62 (m, 1 H), 6.75 (d, J = 8 Hz, 2 H), 6.91 (d, | |
| | | J = 8 Hz, 2 H), 7.16 (d, J = 9 Hz, 1 H), 7.33 (d, J = 8 | |
| | | Hz, 2 H), 7.34-7.37 (m, 1 H), 7.61 (d, J = 8 Hz, 2 | |
| | | H), 11.12 (br s, 1 H) | |
| 14 | B | 1H NMR (400 MHz, DMSO-d6) δ ppm 1.50-1.69 | 496 |
| | | (m, 2 H), 2.10-2.22 (m, 2 H), 2.23-2.30 | |
| | | (m, 2 H), 2.38 (t, J = 7 Hz, 2 H), 2.46-2.61 (m | |
| | | partially hidden, 1 H), 2.64-2.74 (m, 4 H), 3.03 | |
| | | (br t, J = 7 Hz, 2 H), 3.22 (t, J = 7 Hz, 2 H), 3.75 (s, | |
| | | 3 H), 4.42 (dt, J = 48, 6 Hz, 2 H), 6.46 (d, J = 8 Hz, | |
| | | 1 H), 6.50-6.54 (m, 1 H), 6.58-6.62 (m, 1 H), | |
| | | 6.70 (dd, J = 5, 1 Hz, 1 H), 6.80 (d, J = 8 Hz, 2 H), | |
| | | 6.93 (d, J = 8 Hz, 2 H), 7.15 (d, J = 8 Hz, 1 H), 7.35 | |
| | | (t, J = 3 Hz, 1 H), 7.90 (d, J = 5 Hz, 1 H), | |
| | | 11.1-11.14 (m, 1 H) | |
| 15 | B | 1H NMR (400 MHz, DMSO-d6) δ ppm 1.50-1.69 | 496 |
| | | (m, 2 H), 2.11-2.24 (m, 2 H), 2.23-2.30 | |
| | | (m, 2 H), 2.38 (t, J = 7 Hz, 2 H), 2.42-2.60 (m | |
| | | partially hidden, 1 H), 2.65-2.75 (m, 4 H), 3.04 | |
| | | (br t, J = 7 Hz, 2 H), 3.22 (t, J = 7 Hz, 2 H), 3.77 (s, | |
| | | 3 H), 4.42 (dt, J = 48, 6 Hz, 2 H), 6.46 (d, J = 8 Hz, | |
| | | 1 H), 6.56-6.60 (m, 1 H), 6.63 (d, J = 9 Hz, 1 H), | |
| | | 6.79 (d, J = 8 Hz, 2 H), 6.92 (d, J = 8 Hz, 2 H), 7.14 | |
| | | (d, J = 9 Hz, 1 H), 7.34 (t, J = 3 Hz, 1 H), 7.46 (dd, | |
| | | J = 8, 2 Hz, 1 H), 7.90 (d, J = 2 Hz, 1 H), 11.08 (br | |
| | | s, 1 H) | |
| 16 | B | 1H NMR (400 MHz, DMSO-d6) δ ppm 1.52-1.70 | 471 |
| | | (m, 2 H), 1.93-1.99 (m, 2 H), 2.00-2.09 | |
| | | (m, 2 H), 2.11-2.26 (m, 2 H), 2.39 (t, J = 7 Hz, | |
| | | 2 H), 2.60 (m, 1 H), 2.70-2.81 (m, 4 H), 2.93 (br t, | |
| | | J = 7 Hz, 2 H), 3.25 (t, J = 7 Hz, 2 H), 3.54 (t, J = 5 | |
| | | Hz, 2 H), 3.91-3.99 (m, 2 H), 4.43 (dt, J = 48, 6 | |
| | | Hz, 2 H), 5.49-5.52 (m, 1 H), 6.44 (d, J = 8 Hz, 1 | |
| | | H), 6.52-6.56 (m, 1 H), 6.96 (br d, J = 8 Hz, 2 | |
| | | H), 7.03 (br d, J = 8 Hz, 2 H), 7.11 (d, J = 8 Hz, 1 | |
| | | H), 7.31 (t, J = 3 Hz, 1 H), 11.03 (br s, 1 H) | |
| 17 | B | 1H NMR (400 MHz, DMSO-d6) δ ppm 1.54-1.71 | 445 |
| | | (m, 2 H), 2.16-2.24 (m, 4 H), 2.39-2.47 | |
| | | (m partially hidden, 2 H), 2.56-2.68 (m partially | |
| | | hidden, 1 H), 2.74-2.85 (m, 4 H), 2.86-2.95 | |
| | | (m, 2 H), 3.23-3.33 (m partially hidden, 2 H), | |
| | | 3.97 (t, J = 5 Hz, 2 H), 4.44 (dt, J = 48, 6 Hz, 2 H), | |
| | | 4.68 (t, J = 5 Hz, 1 H), 5.93 (dt, J = 16, 5 Hz, 1 H), | |
| | | 6.39 (d, J = 8 Hz, 1 H), 6.46 (d, J = 16 Hz, 1 H), | |
| | | 6.52-6.56 (m, 1 H), 6.95 (d, J = 8 Hz, 2 H), | |
| | | 7.06-7.17 (m, 3 H), 7.31 (t, J = 3 Hz, 1 H), | |
| | | 11.05 (br s, 1 H) | |
| 18 | B | 1H NMR (400 MHz, DMSO-d6) δ ppm 1.15 (s, | 473 |
| | | 6 H), 1.54-1.70 (m, 2 H), 2.14-2.25 (m, 4 H), | |
| | | 2.41 (br t, J = 7 Hz, 2 H), 2.54-2.70 (m partially | |
| | | hidden, 1 H), 2.74-2.79 (m, 2 H), 2.81 (br d, | |
| | | J = 8 Hz, 2 H), 2.85-2.94 (m, 2 H), 3.23-3.33 (m | |
| | | partially hidden, 2 H), 4.43 (dt, J = 48, 6 Hz, 2 H), | |
| | | 4.48 (s, 1 H), 5.94 (d, J = 16 Hz, 1 H), 6.39 (d, J = 8 | |
| | | Hz, 1 H), 6.50 (d, J = 16 Hz, 1 H), 6.53-6.56 (m, | |
| | | 1 H), 6.96 (br d, J = 8 Hz, 2 H), 7.05-7.19 (m, 3 | |
| | | H), 7.31 (m, 1 H), 11.03 (br s, 1 H) | |
| 19 | B | 1H NMR (400 MHz, DMSO-d6) δ ppm 1.50-1.68 | 495 |
| | | (m, 2 H), 2.10-2.23 (m, 2 H), 2.25-2.32 | |
| | | (m, 2 H), 2.38 (t, J = 7 Hz, 2 H), 2.43-2.61 (m | |
| | | partially hidden, 1 H), 2.65-2.71 (m, 4 H), 3.04 | |
| | | (br t, J = 7 Hz, 2 H), 3.21 (t, J = 7 Hz, 2 H), 4.29-4.55 | |
| | | (m, 4 H), 5.09 (t, J = 6 Hz, 1 H), 6.47 (d, J = 8 | |
| | | Hz, 1 H), 6.55-6.60 (m, 1 H), 6.75 (d, J = 8 Hz, 2 | |
| | | H), 6.86 (d, J = 8 Hz, 2 H), 6.95 (br d, J = 6 Hz, 1 | |
| | | H), 7.00-7.10 (m, 2 H), 7.11-7.20 (m, 2 H), | |
| | | 7.33 (t, J = 3 Hz, 1 H), 11.07 (br s, 1 H) | |
| 20 | B | 1H NMR (400 MHz, DMSO-d6) δ ppm 1.50-1.68 | 495 |
| | | (m, 2 H), 2.10-2.22 (m, 2 H), 2.24-2.31 | |
| | | (m, 2 H), 2.38 (t, J = 7 Hz, 2 H), 2.41-2.59 (m | |
| | | partially hidden, 1 H), 2.65-2.72 (m, 4 H), 3.04 | |
| | | (br t, J = 7 Hz, 2 H), 3.22 (t, J = 7 Hz, 2 H), 4.42 | |
| | | (dt, J = 48, 6 Hz, 2 H), 4.41 (d, J = 6 Hz, 2 H), 5.10 | |
| | | (t, J = 6 Hz, 1 H), 6.46 (d, J = 8 Hz, 1 H), 6.56-6.60 | |
| | | (m, 1 H), 6.77 (d, J = 8 Hz, 2 H), 6.87 (d, J = 8 | |
| | | Hz, 2 H), 7.10 (br s, 4 H), 7.14 (d, J = 9 Hz, 1 H), | |
| | | 7.33 (t, J = 3 Hz, 1 H), 11.06 (br s, 1 H) | |
| 21 | B | 1H NMR (400 MHz, DMSO-d6) δ ppm 1.51-1.68 | 509 |
| | | (m, 2 H), 2.11-2.23 (m, 2 H), 2.25-2.32 | |
| | | (m, 2 H), 2.38 (t, J = 7 Hz, 2 H), 2.42-2.60 (m | |
| | | partially hidden, 3 H), 2.62-2.75 (m, 4 H), 3.04 | |
| | | (br t, J = 7 Hz, 2 H), 3.22 (t, J = 7 Hz, 2 H), | |
| | | 3.26-3.40 (m partially hidden, 2 H), 4.42 (dt, J = 48, 6 | |
| | | Hz, 2 H), 4.53 (t, J = 5 Hz, 1 H), 6.47 (d, J = 8 Hz, | |
| | | 1 H), 6.55-6.60 (m, 1 H), 6.73 (d, J = 8 Hz, 2 H), | |
| | | 6.86 (d, J = 8 Hz, 2 H), 6.91-6.96 (m, 2 H), 6.99 | |
| | | (br d, J = 8 Hz, 1 H), 7.04-7.10 (m, 1 H), 7.13 (d, | |
| | | J = 8 Hz, 1 H), 7.33 (t, J = 3 Hz, 1 H), 11,08 (m, 1 | |
| | | H) | |
| 22 | A | 1H NMR (400 MHz, DMSO-d6) δ ppm 1.56-1.75 | 509 |
| | | (m, 2 H), 2.10-2.26 (m, 2 H), 2.29-2.39 | |
| | | (m, 2 H), 2.47-2.76 (m partially hidden, 5 H), | |
| | | 2.84 (t, J = 7 Hz, 2 H), 3.07 (t, J = 7 Hz, 2 H), 3.30 | |
| | | (t, J = 7 Hz, 2 H), 4.45 (dt, J = 48, 6 Hz, 2 H), 6.50 | |
| | | (d, J = 8 Hz, 1 H), 6.59-6.62 (m, 1 H), 6.77 (d, | |
| | | J = 8 Hz, 2 H), 6.89 (d, J = 8 Hz, 2 H), 7.16 (d, J = 9 | |
| | | Hz, 1 H), 7.27 (t, J = 8 Hz, 1 H), 7.35 (br t, J = 3 | |
| | | Hz, 1 H), 7.38 (br d, J = 8 Hz, 1 H), 7.66 (dt, J = 8, | |
| | | 1 Hz, 1 H), 7.68-7.72 (m, 1 H), 11.08 (br s, 1 H) | |
| 23 | A | 1H NMR (400 MHz, DMSO-d6) δ ppm 1.74-1.95 | 509 |
| | | (m, 2 H), 2.14-2.25 (m, 2 H), 2.29-2.38 | |
| | | (m, 2 H), 2.76-2.92 (m, 2 H), 2.92-3.03 (m, 1 H), | |
| | | 3.07 (br t, J = 7 Hz, 2 H), 3.14-3.31 (m, 2 H), | |
| | | 3.70-4.20 (m, 4 H), 4.50 (dt, J = 48, 6 Hz, 2 H), | |
| | | 6.47 (d, J = 8 Hz, 1 H), 6.58-6.62 (m, 1 H), 6.82 | |
| | | (d, J = 8 Hz, 2 H), 6.94 (d, J = 8 Hz, 2 H), 7.16 (br | |
| | | d, J = 8 Hz, 1 H), 7.27 (d, J = 8 Hz, 2 H), 7.36 (br t, | |
| | | J = 3 Hz, 1 H), 7.73 (d, J = 8 Hz, 2 H), 9.67-9.97 | |
| | | (m, 1 H), 11.09 (br s, 1 H), 12.2-13.1 (m, 1 H) | |
| 24 | A | 1H NMR (400 MHz, DMSO-d6) δ ppm 1.55-1.71 | 459 |
| | | (m, 2 H), 2.17-2.30 (m, 4 H), 2.44 (t | |
| | | partially hidden, J = 7 Hz, 2 H), 2.60-2.73 (m, 1 | |
| | | H), 2.77-2.83 (m, 2 H), 2.86 (d, J = 8 Hz, 2 H), | |
| | | 2.89-2.98 (m, 2 H), 3.31 (t partially hidden, J = 7 | |
| | | Hz, 2 H), 4.45 (dt, J = 48, 6 Hz, 2 H), 6.00 (d, | |
| | | J = 16 Hz, 1 H), 6.42 (d, J = 9 Hz, 1 H), 6.58-6.62 | |
| | | (m, 1 H), 6.98 (d, J = 8 Hz, 2 H), 7.15 (d, J = 9 Hz, | |
| | | 1 H), 7.19 (d, J = 8 Hz, 2 H), 7.35-7.37 (m, 1 H), | |
| | | 7.44 (d, J = 16 Hz, 1 H), 11.14 (br s, 1 H) | |
| 25 | D | 1H NMR (400 MHz, DMSO-d6) δ ppm 1.54-1.73 | 447 |
| | | (m, 4 H), 1.92 (br t, J = 7 Hz, 2 H), 2.12-2.28 | |
| | | (m, 4 H), 2.40-2.47 (m partially hidden, 2 H), | |
| | | 2.59-2.70 (m, 1 H), 2.75-2.88 (m, 2 H), 2.81 | |
| | | (d, J = 7 Hz, 2 H), 2.93 (t, J = 7 Hz, 2 H), 3.22-3.43 | |
| | | (m partially hidden , 4 H), 4.34 (t, J = 5 Hz, | |
| | | 1 H), 4.45 (dt, J = 48, 6 Hz, 2 H), 6.40 (d, J = 9 Hz, | |
| | | 1 H), 6.50-6.55 (m, 1 H), 6.97 (d, J = 8 Hz, 2 H), | |
| | | 7.03-7.14 (m, 3 H), 7.25-7.30 (m, 1 H), 10.95 | |
| | | (br s, 1 H) | |
| 26 | C | 1H NMR (400 MHz, DMSO-d6, 27° C.) δ ppm | 515 |
| | | 1.56-1.72 (m, 2 H), 2.05-2.23 (m, 4 H), 2.25 | |
| | | (s, 3 H), 2.40-2.51 (m partially hidden, 2 H), | |
| | | 2.81-2.90 (m, 2 H), 3.01-3.16 (m, 2 H), 3.66 | |
| | | (br t, J = 7 Hz, 2 H), 4.43 (dt, J = 47, 6 Hz, 2 H), | |
| | | 4.65 (quin, J = 6 Hz, 1 H), 6.49 (d, J = 8 Hz, 1 H), | |
| | | 6.54 (d, J = 9 Hz, 2 H), 6.59 (m, 1 H), 6.68 (d, J = 9 | |
| | | Hz, 2 H), 6.97-7.09 (m, 2 H), 7.16 (d, J = 8 Hz, 1 | |
| | | H), 7.23 (dd, J = 8, 1 Hz, 1 H), 7.34 (t, J = 3 Hz, 1 | |
| | | H), 11.09 (br s, 1 H) | |
| 27 | C | 1H NMR (400 MHz, DMSO-d6, 27° C.) δ ppm | 514 |
| | | 1.54-1.71 (m, 2 H), 2.04-2.23 (m, 4 H), 2.23 | |
| | | (s, 3 H), 2.41-2.45 (m partially hidden, 2 H), | |
| | | 2.63-2.71 (m, 2 H), 3.06 (m, 2 H), 3.57 (br t, | |
| | | J = 7 Hz, 2 H), 3.77-3.87 (m, 1 H), 4.43 (dt, | |
| | | J = 47, 6 Hz, 2 H), 5.92 (d, J = 7 Hz, 1 H), 6.19 (d, | |
| | | J = 9 Hz, 2 H), 6.48 (d, J = 9 Hz, 2 H), 6.54 (d, | |
| | | J = 8 Hz, 1 H), 6.56-6.61 (m, 1 H), 6.99-7.10 (m, 2 | |
| | | H), 7.14 (d, J = 8 Hz, 1 H), 7.21 (dd, J = 7, 2 Hz, 1 | |
| | | H), 7.33 (t, J = 3 Hz, 1 H), 11.06 (br s, 1 H) | |
| 28 | C | 1H NMR (400 MHz, DMSO-d6) δ ppm 1.42-1.55 | 528 |
| | | (m, 1 H) 1.69-1.86 (m, 2 H) 2.03-2.31 (m, | |
| | | 6 H) 2.22 (s, 3 H) 2.36-2.52 (m, 3 H) 2.68-2.77 | |
| | | (m, 1 H) 2.97-3.13 (m, 2 H) 3.67-3.80 (m, | |
| | | 1 H) 4.46 (dt, J = 47, 6 Hz, 2 H) 5.60 (d, J = 7 Hz, 1 | |
| | | H) 6.23 (d, J = 9 Hz, 2 H) 6.47 (d, J = 9 Hz, 2 H) | |
| | | 6.52-6.61 (m, 2 H) 7.00-7.10 (m, 2 H) 7.15 (d, | |
| | | J = 9 Hz, 1 H) 7.21 (dd, J = 7, 2 Hz, 1 H) 7.33 (t, | |
| | | J = 3 Hz, 1 H) 11.05-11.10 (m, 1 H) | |
| 29 | C | 1H NMR (400 MHz, DMSO-d6) δ ppm 1.75-1.93 | 525 |
| | | (m, 2 H) 2.06-2.31 (m, 6 H) 2.28 (s, 3H) | |
| | | 2.36-2.65 (m hidden, 4H) 3,05-3.19 (m, 2 H) | |
| | | 3.25-3.40 (m partially hidden, 2H) 4.43 (dt, | |
| | | J = 47, 6 Hz, 2 H) 6.19 (br s, 1 H) 6.51 (br d, J = 8 | |
| | | Hz, 1 H) 6.61 (br s, 1 H) 6.76 (br d, J = 8 Hz, 2 H) | |
| | | 6.94 (br d, J = 8 Hz, 2 H) 6.99-7.11 (m, 2 H) 7.17 | |
| | | (br d, J = 8 Hz, 1 H) 7.23 (br d, J = 6 Hz, 1 H) 7.36 | |
| | | (br s, 1 H) 11.07-11.15 (m, 1 H) | |

The examples which follow describe the preparation of some compounds of formula (I) described herein. The numbers of the compounds exemplified below match those given in the Table 1 above. All reactions are performed under inert atmosphere, unless otherwise stated.

In the following examples, when the source of the starting products is not specified, it should be understood that said products are known compounds.

### INTERMEDIATES

### Intermediate 1: 3-Tosyl-7,8,9,10-tetrahydrocyclohepta[e]indol-6(3H)-one

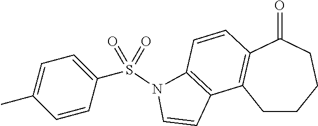 [SMILES: Cc1ccc(S(=O)(=O)n2ccc3c4c(ccc32)C(=O)CCCC4)cc1]

### Step 1: 1-Tosyl-1H-indole-4-carbaldehyde

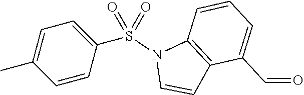 [SMILES: Cc1ccc(S(=O)(=O)n2ccc3c(C=O)cccc32)cc1]

To a mixture of indole-4-carbaldehyde (60 g, 413 mmol) in DCM (100 ml) was added tetrabutylammonium hydrogen sulfate (14 g, 41.3 mmol) and a solution of NaOH (82.7 g, 2.07 mol) in water (80 ml). Then, 4-methylbenzenesulfonyl chloride (86.7 g, 455 mmol) in DCM (200 ml) was added. The mixture was vigorously stirred at RT for 2 h. After decantation, the organic phase was dried over MgSO₄, filtered, and concentrated under reduced pressure. To the residue obtained was purified by flash chromatography, eluting with petroleum ether/EtOAc 70/30 to give 110 g (89%) of 1-tosyl-1H-indole-4-carbaldehyde as a light yellow solid.

LC/MS (m/z, MH+): 300

### Step 2: Ethyl-5-(1-tosyl-1H-indol-4-yl)pent-4-enoate

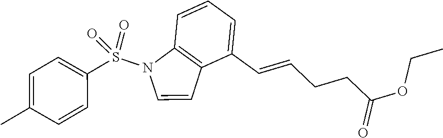 [SMILES: CCOC(=O)CC/C=C/c1cccc2c1ccn2S(=O)(=O)c1ccc(C)cc1]

To a suspension of (4-ethoxy-4-oxobutyl)triphenylphosphonium bromide (252 g, 551 mmol) in anhydrous THF (1350 ml) was dropwise added a 1M solution of potassium bis(trimethylsilyl)amide 1M in THF (551 ml, 826.5 mmol) at −78° C. The mixture was stirred at −78° C. for 1.5 h. A solution of 1-tosyl-1H-indole-4-carbaldehyde (110 g, 367.47 mmol) in anhydrous THF (450 ml) was added. The reaction mixture was stirred at −78° C. for 2 h and the temperature was allowed to warm up to RT. After stirring overnight, the reaction mixture was poured onto a saturated aqueous solution of NH₄Cl and extracted twice with EtOAc. The combined organic layers were dried on MgSO₄ and concentrated under reduced pressure. The residue obtained was purified by flash chromatography, eluting with petroleum ether/EtOAc from 70/30 to give 103 g (71%) of ethyl-5-(1-tosyl-1H-indol-4-yl)pent-4-enoate as a white solid.

LC/MS (m/z, MH+): 398

### Step 3: Ethyl 5-(1-tosyl-1H-indol-4-yl)pentanoate

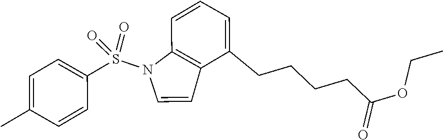 [SMILES: CCOC(=O)CCCCc1cccc2c1ccn2S(=O)(=O)c1ccc(C)cc1]

To a solution of ethyl-5-(1-tosyl-1H-indol-4-yl)pent-4-enoate (35 g, 88.05 mmol) in EtOH (350 ml) in a Paar apparatus was added 10% Pd/C (3.5 g, 1.58 mmol, 10% purity). The mixture was submitted to 6 bars of hydrogen for 2 h then filtered, washed with DCM, and concentrated under reduced pressure. The residue obtained was purified by flash chromatography, eluting with a gradient of petroleum ether/EtOAc from 70/30 to 95/05 to 70/30, to give 24.7 g (66%) of ethyl 5-(1-tosyl-1H-indol-4-yl)pentanoate as a colorless oil.

LC/MS (m/z, MH+): 400

### Step 4: 5-(1-Tosyl-1H-indol-4-yl)pentanoic acid

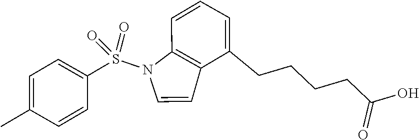 [SMILES: Cc1ccc(S(=O)(=O)n2ccc3c(CCCCC(=O)O)cccc32)cc1]

To a solution of ethyl 5-(1-tosyl-1H-indol-4-yl)pentanoate (64 g, 160.20 mmol) in dioxane (640 ml) was added LiOH (24.9 g, 1.04 mol) in water (200 ml). The mixture was stirred overnight at RT, then poured onto ice-cold aqueous 1N solution of HCl (50 ml) and extracted twice with EtOAc. The combined organic layers were dried on MgSO₄ and concentrated under reduced pressure to give 56 g (79%) of 5-(1-tosyl-1H-indol-4-yl)pentanoic acid.

LC/MS (m/z, MH+): 372

### Step 5: 3-Tosyl-7,8,9,10-tetrahydrocyclohepta[e]indol-6(3H)-one

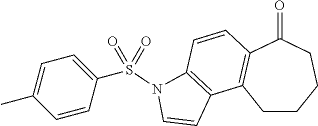 [SMILES: Cc1ccc(S(=O)(=O)n2ccc3c4c(ccc32)C(=O)CCCC4)cc1]

A mixture of 5-(1-tosyl-1H-indol-4-yl)pentanoic acid (56 g, 150.76 mmol) and Eaton's reagent (538 g, 2.26 mol, 354 ml) was heated to 50° C. for 12 h. The mixture is poured onto ice-cold water (3000 ml) and extracted with DCM (600 ml). After decantation, the organic phase was dried over MgSO₄, filtered, and concentrated under reduced pressure. The residue obtained was purified by flash chromatography, eluting with a gradient of heptane/EtOAc from 100/00 to 70/30, to give 10.1 g (18%) of 3-tosyl-7,8,9,10-tetrahydrocyclohepta[e]indol-6(3H)-one.

LC/MS (m/z, MH+): 354

### Intermediate 2: 3-Tosyl-3,8,9,10-tetrahydrocyclohepta[e]indol-6-yl trifluoromethanesulfonate

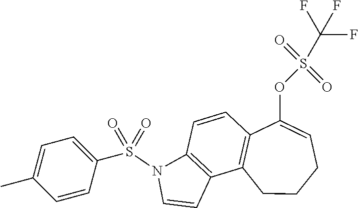 [SMILES: Cc1ccc(S(=O)(=O)n2ccc3c4c(ccc32)C(OS(=O)(=O)C(F)(F)F)=CCCC4)cc1]

To a solution of 3-tosyl-7,8,9,10-tetrahydrocyclohepta[e]indol-6(3H)-one (Intermediate 1) (4.37 g, 12.4 mmol) and pyridine (1.5 ml, 19 mmol) in DCM (120 ml) was added at RT, trifluoromethanesulfonic anhydride (6.79 g, 24.1 mmol). The reaction mixture was stirred at RT for 1 h. The reaction mixture was poured to a mixture of water and ice (100 ml). After decantation, the organic layer was dried over MgSO₄, filtered and concentrated under reduced pressure. The residue was purified by flash chromatography, eluting with heptane/DCM 50/50 to give 5.5 g (91%) of 3-tosyl-3,8,9,10-tetrahydrocyclohepta[e]indol-6-yl trifluoromethanesulfonate.

LC/MS (m/z, MH+): 486

### Intermediate 3: (S)-7-Bromo-6-(4-((1-(3-fluoropropyl)pyrrolidin-3-yl)oxy)phenyl)-3-tosyl-3,8,9,10-tetrahydrocyclohepta[e]indole

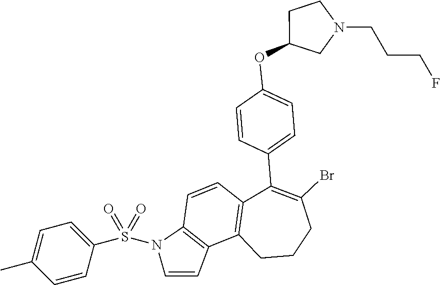 [SMILES: Cc1ccc(S(=O)(=O)n2ccc3c4c(ccc32)C(c2ccc(O[C@H]3CCN(CCCF)C3)cc2)=C(Br)CCC4)cc1]

### Step 1: (S)-6-(4-((1-(3-Fluoropropyl)pyrrolidin-3-yl)oxy)phenyl)-3-tosyl-3,8,9,10-tetrahydrocyclohepta[e]indole

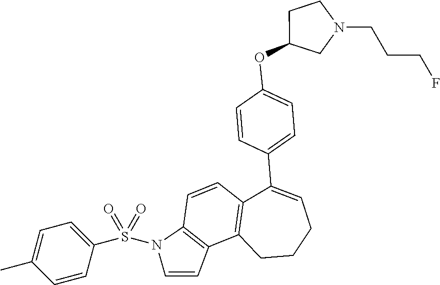 [SMILES: Cc1ccc(S(=O)(=O)n2ccc3c4c(ccc32)C(c2ccc(O[C@H]3CCN(CCCF)C3)cc2)=CCCC4)cc1]

A mixture of 3-tosyl-3,8,9,10-tetrahydrocyclohepta[e]indol-6-yl trifluoromethanesulfonate (Intermediate 2) (940 mg, 2.01 mmol), (S)-1-(3-fluoropropyl)-3-(4-(4,4,5,5-tetramethyl-1,3,2-dioxaborolan-2-yl)phenoxy)pyrrolidine (prepared according to WO2017140669) (772 mg, 2.21 mmol), Cs₂CO₃ (1.38 g, 4.22 mmol) and [1,1′-bis(diphenylphosphino)ferrocene]dichloropalladium(II) (155 mg, 0.2 mmol) in dioxane (40 ml) and water (10 ml) was heated at 60° C. for 1 h. After cooling to RT, the reaction mixture was concentrated under reduced pressure. To the residue obtained, DCM (20 ml) and water (10 ml) were added. After decantation, the organic phase was dried over MgSO₄, filtered and concentrated under reduced pressure. The residue obtained was purified by flash chromatography eluting with a gradient of heptane/EtOAc from 100/00 to 00/100 to give 955 mg (85%) of (S)-6-(4-((1-(3-fluoropropyl)pyrrolidin-3-yl)oxy)phenyl)-3-tosyl-3,8,9,10-tetrahydrocyclohepta[e]indole.

LC/MS (m/z, MH+): 559

### Step 2: (S)-7-Bromo-6-(4-((1-(3-fluoropropyl)pyrrolidin-3-yl)oxy)phenyl)-3-tosyl-3,8,9,10-tetrahydrocyclohepta[e]indole

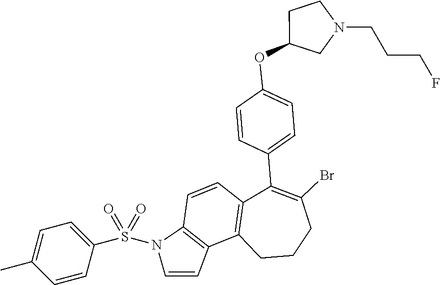 [SMILES: Cc1ccc(S(=O)(=O)n2ccc3c4c(ccc32)C(c2ccc(O[C@H]3CCN(CCCF)C3)cc2)=C(Br)CCC4)cc1]

To a mixture of (S)-6-(4-((1-(3-fluoropropyl)pyrrolidin-3-yl)oxy)phenyl)-3-tosyl-3,8,9,10-tetrahydrocyclohepta[e]indole (50 mg, 0.09 mmol) in DCM (2 ml) cooled at 0° C. was added pyridinium tribromide (31 mg, 0.1 mmol). The mixture was stirred at 5° C. for 1 h. DCM (10 ml) was added followed by a mixture of ice and water (5 ml). After decantation, the organic phase was dried on MgSO₄ and concentrated under reduced pressure to give 45 mg (79%) of (S)-7-bromo-6-(4-((1-(3-fluoropropyl)pyrrolidin-3-yl)oxy)phenyl)-3-tosyl-3,8,9,10-tetrahydrocyclohepta[e]indole.

LC/MS (m/z, MH+): 637

### Intermediate 4: 7-Bromo-6-(4-((1-(3-fluoropropyl)azetidin-3-yl)methyl)phenyl)-3-tosyl-3,8,9,10-tetrahydrocyclohepta[e]indole

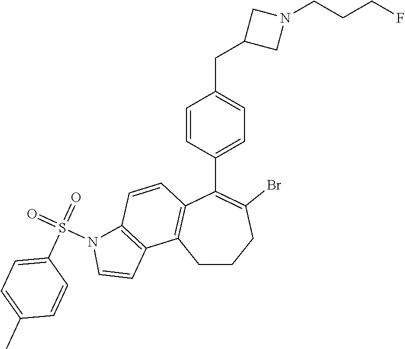 [SMILES: Cc1ccc(S(=O)(=O)n2ccc3c4c(ccc32)C(c2ccc(CC3CN(CCCF)C3)cc2)=C(Br)CCC4)cc1]

### Step 1: 6-(4-((1-(3-Fluoropropyl)azetidin-3-yl)methyl)phenyl)-3-tosyl-3,8,9,10-tetrahydrocyclohepta[e]indole

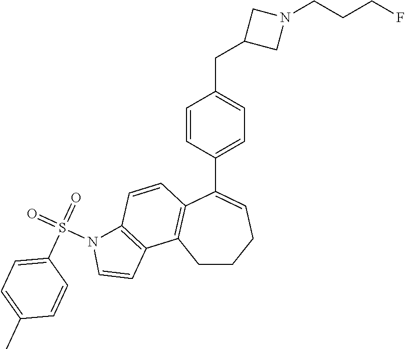 [SMILES: Cc1ccc(S(=O)(=O)n2ccc3c4c(ccc32)C(c2ccc(CC3CN(CCCF)C3)cc2)=CCCC4)cc1]

Step 1 of Intermediate 4 was prepared following a similar procedure to that of Step 1 of Intermediate 3 from 3-tosyl-3,8,9,10-tetrahydrocyclohepta[e]indol-6-yl trifluoromethanesulfonate (Intermediate 2) and 1-(3-fluoropropyl)-3-(4-(4,4,5,5-tetramethyl-1,3,2-dioxaborolan-2-yl)benzyl)azetidine (prepared according to WO2022084280) to give 6.4 g (88%) of 6-(4-((1-(3-fluoropropyl)azetidin-3-yl)methyl)phenyl)-3-tosyl-3,8,9,10-tetrahydrocyclohepta[e]indole.

LC/MS (m/z, MH+): 543

### Step 2: 7-Bromo-6-(4-((1-(3-fluoropropyl)azetidin-3-yl)methyl)phenyl)-3-tosyl-3,8,9,10-tetrahydrocyclohepta[e]indole

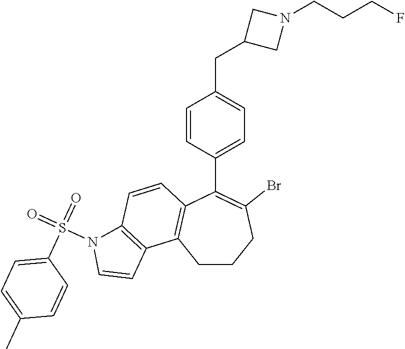 [SMILES: Cc1ccc(S(=O)(=O)n2ccc3c4c(ccc32)C(c2ccc(CC3CN(CCCF)C3)cc2)=C(Br)CCC4)cc1]

Step 2 of Intermediate 4 was prepared following a similar procedure to that of Step 2 of Intermediate 3 from 6-(4-((1-(3-fluoropropyl)azetidin-3-yl)methyl)phenyl)-3-tosyl-3,8,9,10-tetrahydrocyclohepta[e]indole and pyridinium tribromide to give 6 g (82%) of 7-bromo-6-(4-((1-(3-fluoropropyl)azetidin-3-yl)methyl)phenyl)-3-tosyl-3,8,9,10-tetrahydrocyclohepta[e]indole.

LC/MS (m/z, MH+): 621

### Intermediate 5: 7-Bromo-6-(4-((1-(3-fluoropropyl)azetidin-3-yl)methyl)phenyl)-3,8,9,10-tetrahydrocyclohepta[e]indole

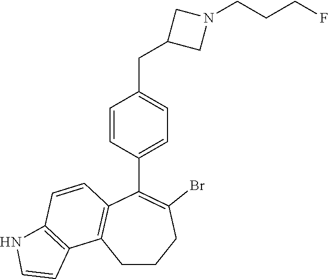 [SMILES: FCCCN1CC(Cc2ccc(C3=C(Br)CCCc4c3ccc3[nH]ccc43)cc2)C1]

To a mixture of 7-bromo-6-(4-((1-(3-fluoropropyl)azetidin-3-yl)methyl)phenyl)-3-tosyl-3,8,9,10-tetrahydrocyclohepta[e]indo (Intermediate 4) (5 g, 8.04 mmol) in MeOH (1 ml), THF (10 ml) and DMSO (10 ml) was added a solution of KOH (2.25 g, 40.1 mmol) in water (2.5 ml). The reaction mixture was stirred at RT for 18 h. Addition of HCl 1N till pH 9. Addition of EtOAc (80 ml) and water (5 ml). After decantation, the organic phase was dried on MgSO₄ and concentrated under reduced pressure and the residue obtained was purified by flash chromatography eluting with a gradient of DCM/MeOH from 97/03 to 95/05 to give 3.44 g (92%) of 7-bromo-6-(4-((1-(3-fluoropropyl)azetidin-3-yl)methyl)phenyl)-3,8,9,10-tetrahydrocyclohepta[e]indole.

LC/MS (m/z, MH+): 467

### Intermediate 6: 7-(3-Chloro-2-methylphenyl)-3-tosyl-3,8,9,10-tetrahydrocyclohepta[e]indol-6-yl trifluoromethanesulfonate

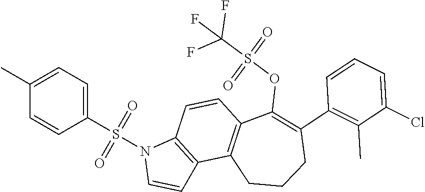 [SMILES: Cc1ccc(S(=O)(=O)n2ccc3c4c(ccc32)C(OS(=O)(=O)C(F)(F)F)=C(c2cccc(Cl)c2C)CCC4)cc1]

### Step 1: 7-(3-Chloro-2-methylphenyl)-3-tosyl-7,8,9,10-tetrahydrocyclohepta[e]indol-6(3H)-one

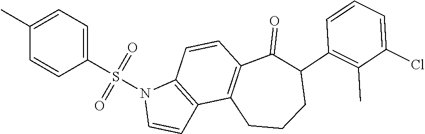 [SMILES: Cc1ccc(S(=O)(=O)n2ccc3c4c(ccc32)C(=O)C(c2cccc(Cl)c2C)CCC4)cc1]

A mixture of 3-tosyl-7,8,9,10-tetrahydrocyclohepta[e]indol-6(3H)-one (Intermediate 1) (6 g, 17 mmol), 1-bromo-3-chloro-2-methyl-benzene (7 g, 34 mmol), Xantphos (1.96 g, 3.4 mmol), Pd₂(dba)₃ (1.55 g, 1.7 mmol), Cs₂CO₃ (17.6 g, 51 mmol) in toluene (90 ml) was heated to reflux for 6 h. After cooling to RT, EtOAc (40 ml) was added and the reaction mixture was filtered through celite. The filtrate was concentrated under reduced pressure and the residue obtained was purified by flash chromatography eluting with DCM to give 4.46 g (55%) of 7-(3-chloro-2-methylphenyl)-3-tosyl-7,8,9,10-tetrahydrocyclohepta[e]indol-6(3H)-one.

LC/MS (m/z, MH+): 478

### Step 2: 7-(3-Chloro-2-methylphenyl)-3-tosyl-3,8,9,10-tetrahydrocyclohepta[e]indol-6-yl trifluoromethanesulfonate

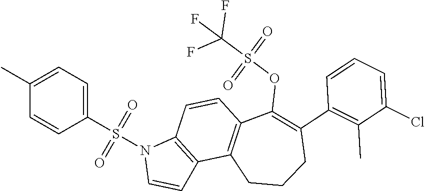 [SMILES: Cc1ccc(S(=O)(=O)n2ccc3c4c(ccc32)C(OS(=O)(=O)C(F)(F)F)=C(c2cccc(Cl)c2C)CCC4)cc1]

To a mixture of 7-(3-chloro-2-methylphenyl)-3-tosyl-7,8,9,10-tetrahydrocyclohepta[e]indol-6(3H)-one (1.44 g, 3 mmol) in THF (25 ml) cooled at −72° C. was added N,N-bis(trifluoromethylsulfonyl)aniline (1.4 g, 3.92 mmol). Then, KHMDS 0.7 M in THF (6 ml, 3.91 mmol) was dropwise added at −72° C. The reaction mixture was stirred at −70° C. for 30 minutes. The cooled bath was removed allowing the temperature to rise to RT. The reaction mixture was stirred at RT for 18 h. To the reaction mixture, EtOAc (50 ml) and water (30 ml) were added. After decantation, the organic phase was dried on MgSO₄ and concentrated under reduced pressure and the residue obtained was purified by flash chromatography eluting with a gradient of heptane/DCM from 100/00 to 60/40 to give 540 mg (29%) of 7-(3-chloro-2-methylphenyl)-3-tosyl-3,8,9,10-tetrahydrocyclohepta[e]indol-6-yl trifluoromethanesulfonate.

LC/MS (m/z, MH+): 610

### Intermediate 7: (3Z)-1-(3-fluoropropyl)-3-[[4-(4,4,5,5-tetramethyl-1,3,2-dioxaborolan-2-yl)phenyl]methylene]pyrrolidine

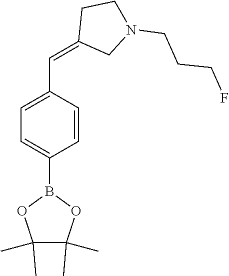 [SMILES: CC1(C)OB(c2ccc(/C=C3/CCN(CCCF)C3)cc2)OC1(C)C]

### Step 1: (4-Bromophenyl)-pyrrolidin-3-yl-methanone, 2,2,2-trifluoroacetic acid

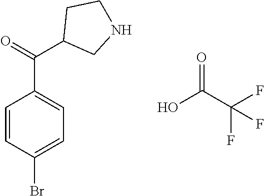 [SMILES: O=C(c1ccc(Br)cc1)C1CCNC1 ; O=C(O)C(F)(F)F]

To a solution of commercially available tert-butyl 3-(4-bromobenzoyl)pyrrolidine-1-carboxylate (20.3 g, 57.31 mmol) in DCM (100 ml) was added dropwise TFA (100 ml) and the reaction mixture was stirred at RT for 30 minutes. The reaction mixture was concentrated under reduced pressure and the residue obtained was triturated with Et₂O. The solid formed was filtered to give 17 g (81%) of (4-bromophenyl)-pyrrolidin-3-yl-methanone, 2,2,2-trifluoroacetic acid.

LC/MS (m/z, MH+): 254

### Step 2: (4-Bromophenyl)-[1-(3-fluoropropyl)pyrrolidin-3-yl]methanone

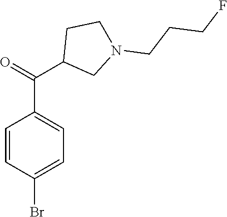 [SMILES: O=C(c1ccc(Br)cc1)C1CCN(CCCF)C1]

A mixture of (4-bromophenyl)-pyrrolidin-3-yl-methanone, 2,2,2-trifluoroacetic acid (9.45 g, 25.67 mmol), K₂CO₃ (10.64 g, 77 mmol), 1-fluoro-3-iodo-propane (5.07 g, 26.95 mmol) in MeCN (200 ml) was heated at 80° C. for 3 hours. The reaction mixture was quenched by addition of a saturated solution of NH₄Cl (200 ml) and extracted with EtOAc (3×200 ml). The organic phase was washed with brine (200 ml), dried over Na₂SO₄ filtered and concentrated under reduced pressure and the residue obtained was purified by flash chromatography, eluting with a gradient of DCM/MeOH: from 100/00 to 50/50 to 90/10 to give 6.34 g (78%) of (4-bromophenyl)-[1-(3-fluoropropyl)pyrrolidin-3-yl]methanone.

LC/MS (m/z, MH+): 314

### Step 3: (4-Bromophenyl)-[1-(3-fluoropropyl)pyrrolidin-3-yl]methanol

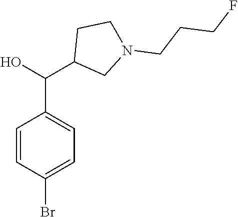 [SMILES: OC(c1ccc(Br)cc1)C1CCN(CCCF)C1]

To a mixture of (4-bromophenyl)-[1-(3-fluoropropyl)pyrrolidin-3-yl]methanone (6.31 g, 20.07 mmol) in MeOH (180 ml) was added NaBH₄ (2.28 g, 60.21 mmol) and the mixture was stirred at RT for 2 hours. A concentrated solution of NH₄Cl (100 ml) and EtOAc (300 ml) were added. After decantation, the organic phase was washed with brine (200 ml), dried over Na₂SO₄ filtered and concentrated under reduced pressure to give 6.69 g (crude) of (4-bromophenyl)-[1-(3-fluoropropyl)pyrrolidin-3-yl]methanol used as such in the next step.

LC/MS (m/z, MH+): 316

### Step 4: 3-[(4-Bromophenyl)methylene]-1-(3-fluoropropyl)pyrrolidine, cis and trans isomers

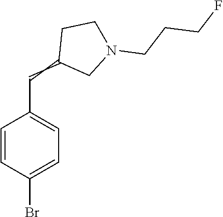 [SMILES: FCCCN1CCC(=Cc2ccc(Br)cc2)C1]

To a mixture of (4-bromophenyl)-[1-(3-fluoropropyl)pyrrolidin-3-yl]methanol (5.16 g, 16.33 mmol) and water (10 ml) at 0° C. was dropwise added sulfuric acid (35 ml). The cooling bath was removed, and the reaction mixture was stirred for 1 hour at RT. The reaction mixture was poured to a mixture of ice and water (500 ml). Powder NaHCO₃ was added to pH 9. EtOAc (300 ml) was added and the organic phase was dried over Na₂SO₄, filtered, concentrated under reduced pressure and the residue obtained was purified by flash chromatography eluting with a gradient of DCM/EtOAc: from 100/00 to 00/100 to give 4.13 g (85%) 3-[(4-bromophenyl)methylene]-1-(3-fluoropropyl)pyrrolidine as a mixture of cis and trans isomers.

### Step 5: (3Z)-1-(3-fluoropropyl)-3-[[4-(4,4,5,5-tetramethyl-1,3,2-dioxaborolan-2-yl)phenyl]methylene]pyrrolidine

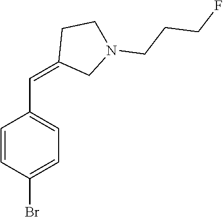 [SMILES: FCCCN1CC/C(=C/c2ccc(Br)cc2)C1]

The mixture of cis and trans isomers of 3-[(4-bromophenyl)methylene]-1-(3-fluoropropyl)pyrrolidine was separated by preparative SFC by using a IF 5×25 cm, 5 μm column (Daicel) and a CO₂ mobile phase with 30% of a 0.1% TEA in MeOH solution as co-solvent. The temperature was set at 40° C. and the back pressure regulator at 100 bars to give 1.15 g of (3Z)-3-[(4-bromophenyl)methylene]-1-(3-fluoropropyl)pyrrolidine and 2.58 g of (3E)-3-[(4-bromophenyl)methylene]-1-(3-fluoropropyl)pyrrolidine.

LC/MS (m/z, MH+): 298

### Step 6: (3Z)-1-(3-fluoropropyl)-3-[[4-(4,4,5,5-tetramethyl-1,3,2-dioxaborolan-2-yl)phenyl]methylene]pyrrolidine

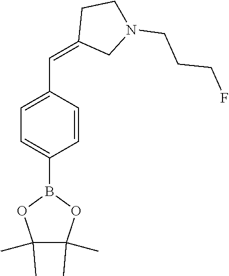 [SMILES: CC1(C)OB(c2ccc(/C=C3/CCN(CCCF)C3)cc2)OC1(C)C]

A mixture of (3Z)-3-[(4-bromophenyl)methylene]-1-(3-fluoropropyl)pyrrolidine (200 mg, 0.67 mmol), in dioxane (4 ml), Pd(PPh₃)₂Cl₂ (38 mg, 0.07 mmol), bis(pinacolato)diboron (221 mg, 0.87 mmol) and AcOK (164 mg, 1.68 mmol) was heated at reflux for 2 hours. After cooling, the reaction mixture was filtered on dicalite. The filtrate was concentrated under reduced pressure and the residue was purified by flash chromatography, eluting with a gradient of DCM/EtOAc/EtOH: from 100/00/00 to 75/20/05 to give 160 mg (69%) of (3Z)-1-(3-fluoropropyl)-3-[[4-(4,4,5,5-tetramethyl-1,3,2-dioxaborolan-2-yl)phenyl]methylene]pyrrolidine.

LC/MS (m/z, MH+): 346

### Intermediate 8: (S)-1-(3-Fluoropropyl)-N-(4-(4,4,5,5-tetramethyl-1,3,2-dioxaborolan-2-yl)phenyl)pyrrolidin-3-amine

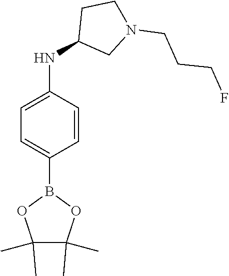 [SMILES: CC1(C)OB(c2ccc(N[C@H]3CCN(CCCF)C3)cc2)OC1(C)C]

### Step 1: (S)—N-(4-Bromophenyl)-1-(3-fluoropropyl)pyrrolidin-3-amine

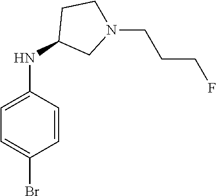 [SMILES: FCCCN1CC[C@H](Nc2ccc(Br)cc2)C1]

A mixture of 1-bromo-4-iodobenzene (2.5 g, 8.8 mmol), Pd(OAc)₂ (200 mg, 0.88 mmol), Xantphos (720 mg, 1.2 mmol), Cs₂CO₃ (8.6 g, 27 mmol) and (3S)-1-(3-fluoropropyl)pyrrolidin-3-amine; bis 2,2,2-trifluoroacetic acid (prepared according to WO2021139756) (6.66 g, 17.8 mmol) in dioxane (60 ml) was heated at reflux for 24 h. After cooling to RT, addition of DCM (120 ml) and water (60 ml). After decantation, the organic phase was dried over MgSO₄, filtered, concentrated under reduced pressure and the residue obtained was purified by flash chromatography eluting with a gradient of DCM/MeOH from 100/00 to 95/05 to give 0.56 g (21%) of (S)—N-(4-bromophenyl)-1-(3-fluoropropyl)pyrrolidin-3-amine.

LC/MS (m/z, MH+): 301

### Step 2: (S)-1-(3-Fluoropropyl)-N-(4-(4,4,5,5-tetramethyl-1,3,2-dioxaborolan-2-yl)phenyl)pyrrolidin-3-amine

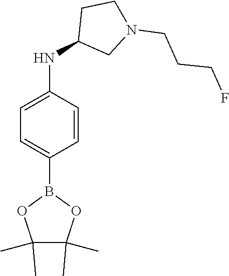 [SMILES: CC1(C)OB(c2ccc(N[C@H]3CCN(CCCF)C3)cc2)OC1(C)C]

Step 2 of Intermediate 8 was prepared following a similar procedure to that of Step 6 of Intermediate 7 from (S)—N-(4-bromophenyl)-1-(3-fluoropropyl)pyrrolidin-3-amine and bis(pinacolato)diboron to give 0.3 g (46%) of (S)-1-(3-fluoropropyl)-N-(4-(4,4,5,5-tetramethyl-1,3,2-dioxaborolan-2-yl)phenyl)pyrrolidin-3-amine.

LC/MS (m/z, MH+): 349

### Intermediate 9: 1-(3-Fluoropropyl)-N-(4-(4,4,5,5-tetramethyl-1,3,2-dioxaborolan-2-yl)phenyl)azetidin-3-amine

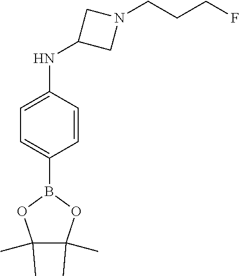 [SMILES: CC1(C)OB(c2ccc(NC3CN(CCCF)C3)cc2)OC1(C)C]

### Step 1: Tert-butyl 3-((4-bromophenyl)amino)azetidine-1-carboxylate

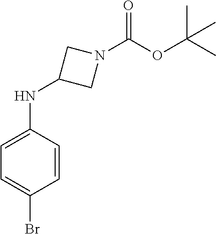 [SMILES: CC(C)(C)OC(=O)N1CC(Nc2ccc(Br)cc2)C1]

Step 1 of Intermediate 10 was prepared following a similar procedure to that of Step 1 of Intermediate 8 from 1-bromo-4-iodobenzene and tert-butyl 3-aminoazetidine-1-carboxylate to give 2.11 g (37%) of tert-butyl 3-((4-bromophenyl)amino)azetidine-1-carboxylate.

LC/MS (m/z, MH+): 327

### Step 2: N-(4-bromophenyl)azetidin-3-amine

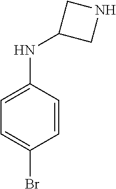 [SMILES: Brc1ccc(NC2CNC2)cc1]

Step 2 of Intermediate 9 was prepared following a similar procedure to that of Step 1 of Intermediate 7 from tert-butyl 3-((4-bromophenyl)amino)azetidine-1-carboxylate to give, after treatment on SCX, 0.88 g (93%) of N-(4-bromophenyl)azetidin-3-amine.

LC/MS (m/z, MH+): 227

### Step 3: N-(4-Bromophenyl)-1-(3-fluoropropyl)azetidin-3-amine

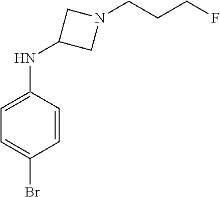 [SMILES: FCCCN1CC(Nc2ccc(Br)cc2)C1]

A mixture of N-(4-bromophenyl)azetidin-3-amine (880 mg, 3.9 mmol), 1-fluoro-3-iodopropane (874 mg, 4.6 mmol) and NaOH (620 mg, 15.5 mmol) in THF (9 ml) was stirred at RT for 24 h. To the reaction mixture, addition of water (5 ml) and EtOAc (10 ml). After decantation, the organic phase was dried over MgSO₄, filtered, concentrated under reduced pressure and the residue obtained was purified by flash chromatography eluting with a gradient of DCM/MeOH from 100/00 to 95/05 to give 338 mg (30%) of N-(4-bromophenyl)-1-(3-fluoropropyl)azetidin-3-amine.

LC/MS (m/z, MH+): 287

### Step 4: 1-(3-Fluoropropyl)-N-(4-(4,4,5,5-tetramethyl-1,3,2-dioxaborolan-2-yl)phenyl)azetidin-3-amine

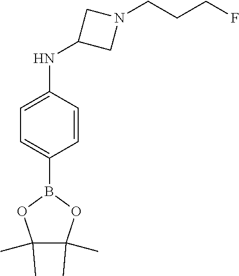 [SMILES: CC1(C)OB(c2ccc(NC3CN(CCCF)C3)cc2)OC1(C)C]

Step 4 of Intermediate 9 was prepared following a similar procedure to that of Step 6 of Intermediate 7 from N-(4-bromophenyl)-1-(3-fluoropropyl)azetidin-3-amine and bis(pinacolato)diboron to give 140 mg (36%) of 1-(3-fluoropropyl)-N-(4-(4,4,5,5-tetramethyl-1,3,2-dioxaborolan-2-yl)phenyl)azetidin-3-amine.

LC/MS (m/z, MH+): 335

### Intermediate 10: 1-(3-Fluoropropyl)-3-(4-(4,4,5,5-tetramethyl-1,3,2-dioxaborolan-2-yl)phenoxy)azetidine

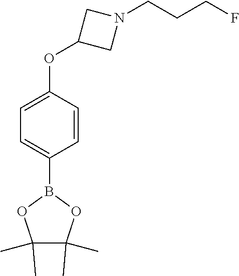 [SMILES: CC1(C)OB(c2ccc(OC3CN(CCCF)C3)cc2)OC1(C)C]

### Step 1: Tert-butyl 3-(4-bromophenoxy)azetidine-1-carboxylate

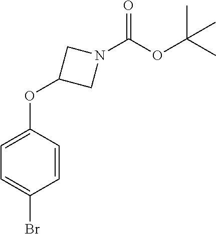 [SMILES: CC(C)(C)OC(=O)N1CC(Oc2ccc(Br)cc2)C1]

A mixture of 4-bromophenol (40 g, 231 mmol), tert-butyl 3-iodoazetidine-1-carboxylate (65.5 g, 231 mmol), Cs₂CO₃ (151 g, 462 mmol) in DMF (400 ml) was stirred at RT for 12 h. To the reaction mixture, water (500 ml) and EtOAc (300 ml) were added. After decantation, the organic phase was dried over MgSO₄, filtered, concentrated under reduced pressure to give 30 g (crude) of tert-butyl 3-(4-bromophenoxy)azetidine-1-carboxylate used as such in the next step.

LC/MS (m/z, MH+): 328

### Step 2: 3 3-(4-Bromophenoxy)azetidine, 2,2,2-trifluoroacetic acid

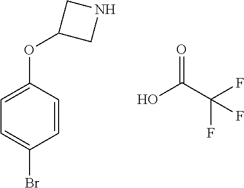 [SMILES: Brc1ccc(OC2CNC2)cc1 ; O=C(O)C(F)(F)F]

Step 2 of Intermediate 10 was prepared following a similar procedure to that of Step 1 of Intermediate 7 from tert-butyl 3-(4-bromophenoxy)azetidine-1-carboxylate to give 20 g (crude) of 3-(4-bromophenoxy)azetidine, 2,2,2-trifluoroacetic acid used as such in the next step.

LC/MS (m/z, MH+): 228

### Step 3: 3-(4-Bromophenoxy)-1-(3-fluoropropyl)azetidine

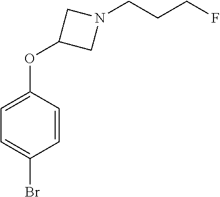 [SMILES: FCCCN1CC(Oc2ccc(Br)cc2)C1]

A mixture of 3-(4-bromophenoxy)azetidine, 2,2,2-trifluoroacetic acid (20 g, 58.48 mmol), 1-fluoro-3-iodopropane (11 g, 58.48 mmol) and KOH (6.54 g, 117 mmol) in DMF (100 ml) was stirred at RT for 12 h. To the reaction mixture wax concentrated under reduced pressure and the residue obtained was purified by flash chromatography eluting with a gradient of petroleum ether/EtOAc from 97/03 to 00/100 to give 9 g (53%) of N-(4-3-(4-bromophenoxy)-1-(3-fluoropropyl)azetidine.

LC/MS (m/z, MH+): 288

### Step 4: 1-(3-Fluoropropyl)-3-(4-(4,4,5,5-tetramethyl-1,3,2-dioxaborolan-2-yl)phenoxy)azetidine

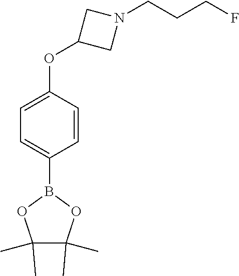 [SMILES: CC1(C)OB(c2ccc(OC3CN(CCCF)C3)cc2)OC1(C)C]

Step 4 of Intermediate 10 was prepared following a similar procedure to that of Step 6 of Intermediate 7 from 3-(4-bromophenoxy)-1-(3-fluoropropyl)azetidine and bis(pinacolato)diboron to give 140 mg (36%) of 1-(3-fluoropropyl)-3-(4-(4,4,5,5-tetramethyl-1,3,2-dioxaborolan-2-yl)phenoxy)azetidine.

LC/MS (m/z, MH+): 336

### EXAMPLES

### Method A

### Example 1: 7-(4-Fluoro-2-methylphenyl)-6-(4-((1-(3-fluoropropyl)azetidin-3-yl)methyl)phenyl)-3,8,9,10-tetrahydrocyclohepta[e]indole

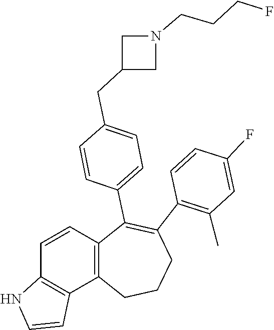 [SMILES: Cc1cc(F)ccc1C1=C(c2ccc(CC3CN(CCCF)C3)cc2)c2ccc3[nH]ccc3c2CCC1]

### Step 1: 7-(4-Fluoro-2-methylphenyl)-6-(4-((1-(3-fluoropropyl)azetidin-3-yl)methyl)phenyl)-3-tosyl-3,8,9,10-tetrahydrocyclohepta[e]indole

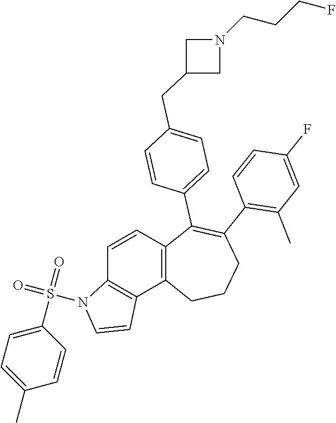 [SMILES: Cc1ccc(S(=O)(=O)n2ccc3c4c(ccc32)C(c2ccc(CC3CN(CCCF)C3)cc2)=C(c2ccc(F)cc2C)CCC4)cc1]

A mixture of 7-bromo-6-(4-((1-(3-fluoropropyl)azetidin-3-yl)methyl)phenyl)-3-tosyl-3,8,9,10-tetrahydrocyclohepta[e]indole (Intermediate 4) (150 mg, 0.24 mmol), (4-fluoro-2-methyl-phenyl)boronic acid (74 mg, 0.48 mmol), Cs₂CO₃ (165 mg, 0.51 mmol) and [1,1′-bis(diphenylphosphino)ferrocene]dichloropalladium(II) (18 mg, 0.024 mmol) in dioxane (8 ml) and water (2 ml) was heated at 90° C. for 30 minutes. After cooling to RT, EtOAc (200 ml) and water (50 ml) were added. After decantation, the organic phase was dried over MgSO₄, filtered and concentrated under reduced pressure. The residue obtained was purified by flash chromatography eluting with a gradient of cyclohexane/EtOAc from 100/00 to 00/100 to give 106 mg (65%) of 7-(4-fluoro-2-methylphenyl)-6-(4-((1-(3-fluoropropyl)azetidin-3-yl)methyl)phenyl)-3-tosyl-3,8,9,10-tetrahydrocyclohepta[e]indole.

LC/MS (m/z, MH+): 651

### Step 2: 7-(4-Fluoro-2-methylphenyl)-6-(4-((1-(3-fluoropropyl)azetidin-3-yl)methyl)phenyl)-3,8,9,10-tetrahydrocyclohepta[e]indole

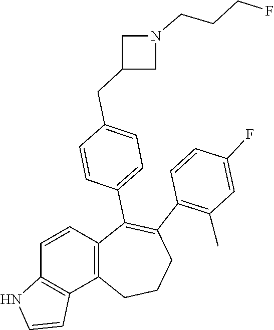 [SMILES: Cc1cc(F)ccc1C1=C(c2ccc(CC3CN(CCCF)C3)cc2)c2ccc3[nH]ccc3c2CCC1]

A mixture of 7-(4-fluoro-2-methylphenyl)-6-(4-((1-(3-fluoropropyl)azetidin-3-yl)methyl)phenyl)-3-tosyl-3,8,9,10-tetrahydrocyclohepta[e]indole (106 mg, 0.16 mmol) and KOH (46 mg, 0.81 mmol) in EtOH (10 ml) and water (2 ml) was heated at 70° C. for 24 h. After cooling to RT, HCl 1N was added down to pH 7. Then, EtOAc (50 ml), ethyl ether (50 ml) and water (20) were added. After decantation, the organic phase was dried over MgSO₄, filtered and concentrated under reduced pressure. The residue obtained was purified by flash chromatography eluting with a gradient of DCM/MeOH from 100/00 to 80/20 to give 58 mg (72%) of 7-(4-fluoro-2-methylphenyl)-6-(4-((1-(3-fluoropropyl)azetidin-3-yl)methyl)phenyl)-3,8,9,10-tetrahydrocyclohepta[e]indole.

### Method B

### Example 2: 7-(3-Fluoro-2-methoxypyridin-4-yl)-6-(4-((1-(3-fluoropropyl)azetidin-3-yl)methyl)phenyl)-3,8,9,10-tetrahydrocyclohepta[e]indole

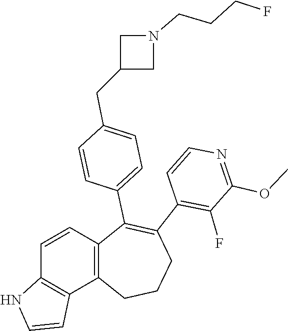 [SMILES: COc1nccc(C2=C(c3ccc(CC4CN(CCCF)C4)cc3)c3ccc4[nH]ccc4c3CCC2)c1F]

Example 2 was prepared following a similar procedure to that of Step 1 of Example 1 from 7-bromo-6-(4-((1-(3-fluoropropyl)azetidin-3-yl)methyl)phenyl)-3,8,9,10-tetrahydrocyclohepta[e]indole (Intermediate 5) and (3-fluoro-2-methoxy-4-pyridyl)boronic acid to give 81 mg (67%) of 7-(3-fluoro-2-methoxypyridin-4-yl)-6-(4-((1-(3-fluoropropyl)azetidin-3-yl)methyl)phenyl)-3,8,9,10-tetrahydrocyclohepta[e]indole.

### Example 17: (E)-3-(6-(4-((1-(3-fluoropropyl)azetidin-3-yl)methyl)phenyl)-3,8,9,10-tetrahydrocyclohepta[e]indol-7-yl)prop-2-en-1-ol

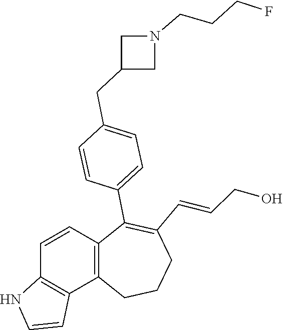 [SMILES: OC/C=C/C1=C(c2ccc(CC3CN(CCCF)C3)cc2)c2ccc3[nH]ccc3c2CCC1]

Example 17 was prepared following a similar procedure to that of Step 1 of Example 1 from 7-bromo-6-(4-((1-(3-fluoropropyl)azetidin-3-yl)methyl)phenyl)-3,8,9,10-tetrahydrocyclohepta[e]indole (Intermediate 5) and (E)-3-(4,4,5,5-tetramethyl-1,3,2-dioxaborolan-2-yl)prop-2-en-1-ol to give 119 mg (50%) of (E)-3-(6-(4-((1-(3-fluoropropyl)azetidin-3-yl)methyl)phenyl)-3,8,9,10-tetrahydrocyclohepta[e]indol-7-yl)prop-2-en-1-ol.

### Method C

### Example 3: 7-(3-Chloro-2-methylphenyl)-6-(4-((1-(3-fluoropropyl)azetidin-3-ylidene)methyl)phenyl)-3,8,9,10-tetrahydrocyclohepta[e]indole

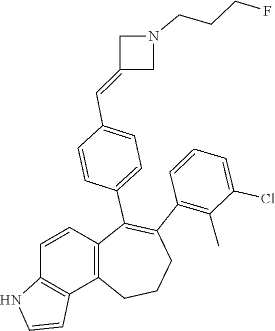 [SMILES: Cc1c(Cl)cccc1C1=C(c2ccc(C=C3CN(CCCF)C3)cc2)c2ccc3[nH]ccc3c2CCC1]

### Step 1: 7-(3-Chloro-2-methylphenyl)-6-(4-((1-(3-fluoropropyl)azetidin-3-ylidene)methyl)phenyl)-3-tosyl-3,8,9,10-tetrahydrocyclohepta[e]indole

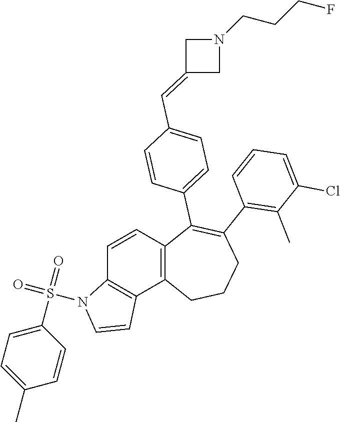 [SMILES: Cc1ccc(S(=O)(=O)n2ccc3c4c(ccc32)C(c2ccc(C=C3CN(CCCF)C3)cc2)=C(c2cccc(Cl)c2C)CCC4)cc1]

Step 1 of Example 3 was prepared following a similar procedure to that of Step 1 of Example 1 from 7-(3-chloro-2-methylphenyl)-3-tosyl-3,8,9,10-tetrahydrocyclohepta[e]indol-6-yl trifluoromethanesulfonate (Intermediate 6) and 1-(3-fluoropropy))-3-(4-(4,4,5,5-tetramethyl-1,3,2-dioxaborolan-2-yl)benzylidene)azetidine (according to WO2022084298) to give 180 mg (61%) of 7-(3-chloro-2-methylphenyl)-6-(4-((1-(3-fluoropropyl)azetidin-3-ylidene)methyl)phenyl)-3-tosyl-3,8,9,10-tetrahydrocyclohepta[e]indole.

LC/MS (m/z, MH+): 665

### Step 2: 7-(3-Chloro-2-methylphenyl)-6-(4-((1-(3-fluoropropyl)azetidin-3-ylidene)methyl)phenyl)-3,8,9,10-tetrahydrocyclohepta[e]indole

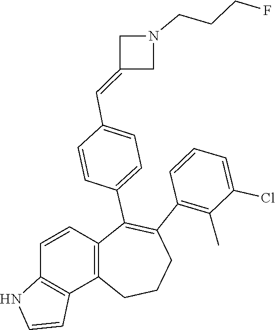 [SMILES: Cc1c(Cl)cccc1C1=C(c2ccc(C=C3CN(CCCF)C3)cc2)c2ccc3[nH]ccc3c2CCC1]

Step 2 of Example 3 was prepared following a similar procedure to that of Step 2 of Example 1 from of 7-(3-chloro-2-methylphenyl)-6-(4-((1-(3-fluoropropyl)azetidin-3-ylidene)methyl)phenyl)-3-tosyl-3,8,9,10-tetrahydrocyclohepta[e]indole to give 125 mg (90%) of 7-(3-chloro-2-methylphenyl)-6-(4-((1-(3-fluoropropyl)azetidin-3-ylidene)methyl)phenyl)-3,8,9,10-tetrahydrocyclohepta[e]indole.

### Method D

### Example 25: 3-(6-(4-((1-(3-Fluoropropyl)azetidin-3-yl)methyl)phenyl)-3,8,9,10-tetrahydrocyclohepta[e]indol-7-yl)propan-1-ol

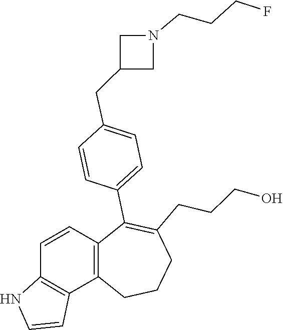 [SMILES: OCCCC1=C(c2ccc(CC3CN(CCCF)C3)cc2)c2ccc3[nH]ccc3c2CCC1]

A mixture of (E)-3-(6-(4-((1-(3-fluoropropyl)azetidin-3-yl)methyl)phenyl)-3,8,9,10-tetrahydrocyclohepta[e]indol-7-yl)prop-2-en-1-ol (Example 17) (100 mg, 0.22 mmol), Pd/C 10% (24 mg, 0.024 mmol) in EtOAc (5 ml) and EtOH (8 ml) was hydrogenated under H₂ (1 bar) at RT for 15 h. The reaction mixture was filtered on celite and the filtrate concentrated under reduced pressure. The residue obtained was purified by flash chromatography eluting with a gradient of DCM/MeOH from 100/00 to 90/10 to give 70 mg (70%) of 3-(6-(4-((1-(3-fluoropropyl)azetidin-3-yl)methyl)phenyl)-3,8,9,10-tetrahydrocyclohepta[e]indol-7-yl)propan-1-ol.

The compounds according to table 1 above were subjected to pharmacological tests for determining their degradation effects on estrogen receptors.

Test: Estrogen receptor degradation activity

Said test involves measuring the in vitro degradation activity of the compounds of the table 1.

The measurements of the degradation activities were made using a breast cancer cell ERα in cell western assay as described hereunder.

MCF7 cells (ATCC) were seeded in 384 wells microplate (collagen coated) at a concentration of 10000 cells/30 μL per well in red phenol free MEM alpha medium (invitrogen) containing 5% charcoal dextran striped FBS. The following day, 9 points serial 1:5 dilution of each compound was added to the cells in 2.5 μL at final concentrations ranging from 0.3-0.0000018 μM (in table 2), or 0.1 μM for fulvestrant (using as positive control). At 4 hours post compound addition the cells were fixed by adding 25 μL of formalin (final concentration 5% formalin containing 0.1% triton) for 10 minutes at RT and then washed twice with PBS. Then, 50 μL of LI-COR blocking buffer containing 0.1% Triton was added to plate for 30 minutes at RT. LI-COR blocking buffer was removed and cells were incubated overnight at cold room with 50 μL anti-ER rabbit monoclonal antibody (Thermo scientific MA1-39540) diluted at 1:1000 in LI-COR blocking buffer containing 0.1% tween-20. Wells which were treated with blocking buffer but no antibody were used as background control. Wells were washed twice with PBS (0.1% tween-20) and incubated at 37° C. for 60 minutes in LI-COR (0.1% tween-20) containing goat anti-rabbit antibody Alexa 488 (1:1000) and Syto-64 a DNA dye (2 μM final concentration). Cells were then washed 3 times in PBS and scanned in ACUMEN explorer (TTP-Labtech). Integrated intensities in the green fluorescence and red fluorescence were measured to determine the levels of ERα and DNA respectively.

The degradation activity with respect to estrogen receptors in this test is given by the concentration which degrades 50% of the estrogen receptor (or IC₅₀) in nM.

The % of ERα levels decrease were determined as follows: % inhibition=100*(1−(sample−fulvestrant:DMSO−fulvestrant)).

The Table 2 below indicates the estrogen receptor degradation activity results for the compounds of table 1 tested at 0.3 μM, and demonstrates that said compounds have a significant degradation activity on estrogen receptors.

TABLE 2

| | Degradation | % Degradation |
|---|---|---|
| Compound No. | IC₅₀ (nM) | At 0.3 μM |
| 1 | 2 | 80 |
| 2 | 0.3 | 92 |
| 3 | 2 | 91 |
| 4 | 2 | 85 |
| 5 | 3 | 95 |
| 6 | 1 | 90 |
| 7 | 3 | 82 |
| 8 | 1 | 83 |
| 9 | 0.4 | 80 |
| 10 | 2 | 73 |
| 11 | 0.5 | 75 |
| 12 | 0.4 | 85 |
| 13 | 1 | 72 |
| 14 | 0.3 | 91 |
| 15 | 0.3 | 91 |
| 16 | 0.4 | 74 |
| 17 | 1 | 69 |
| 18 | 0.5 | 77 |
| 19 | 0.3 | 71 |
| 20 | 0.2 | 76 |
| 21 | 0.5 | 65 |
| 22 | 54 | 68 |
| 23 | 26 | 80 |
| 24 | 37 | 92 |
| 25 | 0.4 | 75 |
| 26 | 2 | 91 |
| 27 | 3 | 91 |
| 28 | 2 | 91 |
| 29 | 11 | 91 |

It is therefore apparent that the tested compounds have degradation activities for estrogen receptors, with IC50 less than 1 μM and with degradation levels greater than 50%. The compounds of formula (I) can therefore be used for preparing medicaments, especially medicaments which are degraders of estrogen receptors.

Accordingly, also provided herein are medicaments which comprise a compound of the formula (I), or a pharmaceutically acceptable salt thereof.

Herein are also provided the compounds of formula (I) defined above, or pharmaceutically acceptable salts thereof, for use as medicines.

Herein are also provided the compounds of formula (I) defined above, or pharmaceutically acceptable salt thereof, for use in therapy, especially as inhibitors and degraders of estrogen receptors.

Herein are also provided the compounds of formula (I) defined above, or a pharmaceutically acceptable salts thereof, for use in the treatment of ovulatory dysfunction, cancer, endometriosis, osteoporosis, benign prostatic hypertrophy or inflammation.

A particular aspect is a compound of formula (I) defined above, or a pharmaceutically acceptable salt thereof, for use in the treatment of cancer.

In an embodiment, the cancer is a hormone dependent cancer.

In another embodiment, the cancer is an estrogen receptor dependent cancer, particularly the cancer is an estrogen receptor a dependent cancer.

In another embodiment, the cancer is selected from breast, ovarian, endometrial, prostate, uterine, cervical and lung cancer, or a metastasis thereof.

In another embodiment, the metastasis is a cerebral metastasis.

In another embodiment, the cancer is breast cancer. Particularly, the breast cancer is an estrogen receptor positive breast cancer (ERα positive breast cancer).

In another embodiment, the cancer is resistant to anti-hormonal treatment.

In a further embodiment, the compound of formula (I) is as used as single agent or in combination with other agents such as CDK4/6, mTOR or PI3K inhibitors.

According to another aspect, herein is provided a method of treating the pathological conditions indicated above, comprising administering to a subject in need thereof a therapeutically effective amount of a compound of formula (I), or a pharmaceutically acceptable salt thereof. In an embodiment of this method of treatment, the subject is a human.

Herein is also provided the use of a compound of the formula (I), or a pharmaceutically acceptable salt thereof, for the manufacture of a medicament useful in treating any of the pathological conditions indicated above, more particularly useful in treating cancer.

Herein are also provided the pharmaceutical compositions comprising as active principle a compound of formula (I). These pharmaceutical compositions comprise an effective dose of at least one compound of formula (I), or a pharmaceutically acceptable salt thereof, and also at least one pharmaceutically acceptable excipient.

The said excipients are selected, in accordance with the pharmaceutical form and method of administration desired, from the customary excipients, which are known to a person skilled in the art.

In the pharmaceutical compositions for oral, sublingual, subcutaneous, intramuscular, intravenous, topical, local, intra-tracheal, intranasal, transdermal or rectal administration, the active principle of formula (I) above, or its base, acid, zwitterion or salt thereof, may be administered in a unit administration form, in a mixture with conventional pharmaceutical excipients, to animals and to human beings for the treatment of the above disorders or diseases.

The unit administration forms appropriate include oral forms such as tablets, soft or hard gel capsules, powders, granules and oral solutions or suspensions, sublingual, buccal, intra-tracheal, intra-ocular and intra-nasal administration forms, forms for inhalative, topical, transdermal, subcutaneous, intra-muscular or intravenous administration, rectal administration forms and implants. For topical application it is possible to use the compounds of formula (I) in creams, gels, ointments or lotions.

As an example, a unit administration form of a compound of formula (I) in tablet form may comprise the following components:

| | | |
|---|---|---|
| Compound of formula (I) | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

There may be particular cases in which higher or lower dosages are appropriate. According to usual practice, the dosage that is appropriate for each patient is determined by the doctor according to the mode of administration and the weight and response of the said patient.

1. A compound of the formula (I) or a pharmaceutically acceptable salt thereof: 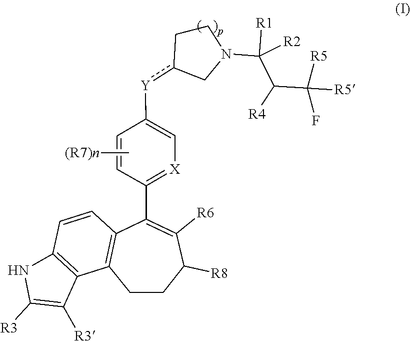
wherein:
R1 and R2 independently represent a hydrogen atom or a deuterium atom;
R3 and R3′ represent a hydrogen atom, or a fluorine atom;
R4 represents a hydrogen atom or a fluorine atom;
R5 and R5′ independently represent a hydrogen atom or a fluorine atom;
Y represents —CH₂—, —CH═, —CR9=, —O— or —NH—, wherein R9 represents a fluorine atom or a (C₁-C₃)alkyl group; [symbol]
represents a single bond or a double bond;
p is 0 or 1;
X represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl;
R6 represents a group selected from:
a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; a —COOH group and a —OH group;
a fused phenyl group, selected from phenyl groups fused with a (C₃-C₆)cycloalkyl, which (C₃-C₆)cycloalkyl ring optionally comprises an unsaturation and, wherein the fused phenyl group is optionally substituted with 1 to 3 substituents independently selected from a (C₁-C₃) alkyl group, a hydroxy group, a halogen atom, a (C₁-C₆)fluoroalkyl group and a (C₁-C₃)alkoxy group;
a phenyl group fused with a hetero(C₄-C₆)cycloalkyl, which hetero(C₄-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)-alkyl group, a (C₁-C₃)fluoroalkyl group, a (C₁-C₃)alkoxy group, a (C₁-C₃)fluoroalkoxy group and an oxo group;
a heteroaryl group comprising 2 to 9 carbon atoms and comprising from 1 to 3 heteroatoms independently selected from oxygen, nitrogen and sulfur, and at least 5 atoms including carbon atoms and heteroatoms, such as a pyridyl group, a pyridone group or a pyrrolyl group, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
a (C₃-C₆)cycloalkyl group, and a phenyl group, said (C₃-C₆)cycloalkyl or phenyl groups being optionally substituted with one or two halogen atom(s) or (C₁-C₃)alkyl group(s);
a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
a 4 to 7 membered-heterocycloalkyl group comprising 1 or 2 heteroatoms independently selected from oxygen, nitrogen and sulfur, such as a tetrahydropyranyl, a dihydropyran or a tetrahydrofuranyl group, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
a (C₁-C₆)alkyl group, such as an isobutyl group or an ethylbutyl group, said alkyl group being optionally substituted with 1 to 4 substituents independently selected from: a fluorine atom, a (C₁-C₃)alkoxy group, a (C₁-C₃)fluoroalkoxy group and a —OH group;
a (C₁-C₆)alkenyl group, said (C₁-C₆)alkenyl group being optionally substituted with 1 to 4 substituents independently selected from: a —OH group; a halogen atom; a (C₁-C₃)alkyl group; a (C₁-C₃)fluoroalkyl group; a (C₁-C₃)alkoxy group; a (C₁-C₃) fluoroalkoxy group; a —COOH group and a cyano group; and
a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃) fluoroalkoxy group; a cyano group; and a —OH group;
R7 independently represents a (C₁-C₃)alkyl group, such as a methyl group, a halogen atom, such as a fluorine atom, a cyano group, or a (C₁-C₃)fluoroalkyl group, such as a trifluoromethyl;
R8 represents a hydrogen atom or a (C₁-C₃)alkyl group or a cyclopropyl; and
n is 0, 1 or 2.

2. The compound of formula (I) according to claim 1, or a pharmaceutically acceptable salt thereof, characterized in that R1 and R2 are a hydrogen atom.

3. The compound of formula (I) according to claim 1 or 2, or a pharmaceutically acceptable salt thereof, characterized in that R3 and R3′ are a hydrogen atom.

4. The compound of formula (I) according to anyone of claims 1 to 3, or a pharmaceutically acceptable salt thereof, characterized in that R4, R5 and R5′ represent a hydrogen atom.

5. The compound of formula (I) according to anyone of claims 1 to 4, or a pharmaceutically acceptable salt thereof, characterized in that X represents —CH═.

6. The compound of formula (I) according to any one of claims 1 to 5, or a pharmaceutically acceptable salt thereof, characterized in that Y is —CH₂—, —CH═, —O— or —NH—.

7. The compound of formula (I) according to any one of claims 1 to 6, or a pharmaceutically acceptable salt thereof, characterized in that R7 represents a hydrogen atom and n is 1.

8. The compound of formula (I) according to anyone of claims 1 to 7, characterized in that R6 represents a phenyl group, said phenyl group being optionally substituted by 1 to 3 substituents independently selected from a fluorine atom; a chlorine atom; a (C₁-C₄)alkyl group, such as a methyl or ethyl group, optionally substituted with a OH group; a trifluoromethyl group; a (C₁-C₄)alkoxy group, such as a methoxy group; a cyano group; a —COOH group and a —OH group.

9. The compound of formula (I) according to anyone of claims 1 to 7, characterized in that R6 represents a pyridyl group, said pyridyl group being optionally substituted with 1 to 3 substituents independently selected from a fluorine atom and a (C₁-C₆)alkoxy group, more particularly a methoxy group.

10. The compound of formula (I) according to anyone of claims 1 to 7, characterized in that R6 represents a saturated or partially saturated cyclohexyl group, said cyclohexyl group being optionally substituted with 1 to 2 fluorine atoms.

11. The compound of formula (I) according to anyone of claims 1 to 7, characterized in that R6 represents a saturated or partially saturated 6-membered heterocycloalkyl group comprising an oxygen atom, such as a dihydropyranyl.

12. The compound of formula (I) according to anyone of claims 1 to 11, wherein R8 represents a hydrogen atom.

13. The compound of formula (I) according to anyone of claims 1 to 12, or a pharmaceutically acceptable salt thereof, in particular trifluoroacetate thereof, characterized in that said compound is selected from the following compounds:
7-(4-fluoro-2-methylphenyl)-6-(4-((1-(3-fluoropropyl)azetidin-3-yl)methyl)phenyl)-3,8,9,10-tetrahydrocyclohepta[e]indole (1),
7-(3-fluoro-2-methoxypyridin-4-yl)-6-(4-((1-(3-fluoropropyl)azetidin-3-yl)methyl)phenyl)-3,8,9,10-tetrahydrocyclohepta[e]indole (2),
7-(3-chloro-2-methylphenyl)-6-(4-((1-(3-fluoropropyl)azetidin-3-ylidene)methyl)phenyl)-3,8,9,10-tetrahydrocyclohepta[e]indole (3),
(S)-7-(2,4-dichlorophenyl)-6-(4-((1-(3-fluoropropyl)pyrrolidin-3-yl)oxy)phenyl)-3,8,9,10-tetrahydrocyclohepta[e]indole (4),
(S)-7-(4,4-difluorocyclohex-1-en-1-yl)-6-(4-((1-(3-fluoropropyl)pyrrolidin-3-yl)oxy)phenyl)-3,8,9,10-tetrahydrocyclohepta[e]indole (5),
2,6-difluoro-3-(6-(4-((1-(3-fluoropropyl)azetidin-3-yl)methyl)phenyl)-3,8,9,10-tetrahydrocyclohepta[e]indol-7-yl)phenol (6),
7-(2,4-dichlorophenyl)-6-(4-((1-(3-fluoropropyl)azetidin-3-yl)methyl)phenyl)-3,8,9,10-tetrahydrocyclohepta[e]indole (7),
7-(3-chloro-2-methylphenyl)-6-(4-((1-(3-fluoropropyl)azetidin-3-yl)methyl)phenyl)-3,8,9,10-tetrahydrocyclohepta[e]indole (8),
7-(2,3-dimethoxyphenyl)-6-(4-((1-(3-fluoropropyl)azetidin-3-yl)methyl)phenyl)-3,8,9,10-tetrahydrocyclohepta[e]indole (9),
6-(4-((1-(3-fluoropropyl)azetidin-3-yl)methyl)phenyl)-7-(3-(trifluoromethyl)phenyl)-3,8,9,10-tetrahydrocyclohepta[e]indole (10),
3-(6-(4-((1-(3-fluoropropyl)azetidin-3-yl)methyl)phenyl)-3,8,9,10-tetrahydrocyclohepta[e]indol-7-yl)benzonitrile (11),
2-(6-(4-((1-(3-fluoropropyl)azetidin-3-yl)methyl)phenyl)-3,8,9,10-tetrahydrocyclohepta[e]indol-7-yl)benzonitrile (12),
4-(6-(4-((1-(3-fluoropropyl)azetidin-3-yl)methyl)phenyl)-3,8,9,10-tetrahydrocyclohepta[e]indol-7-yl)benzonitrile (13),
6-(4-((1-(3-fluoropropyl)azetidin-3-yl)methyl)phenyl)-7-(2-methoxypyridin-4-yl)-3,8,9,10-tetrahydrocyclohepta[e]indole (14),
6-(4-((1-(3-fluoropropyl)azetidin-3-yl)methyl)phenyl)-7-(6-methoxypyridin-3-yl)-3,8,9,10-tetrahydrocyclohepta[e]indole (15)
7-(3,6-dihydro-2H-pyran-4-yl)-6-(4-((1-(3-fluoropropyl)azetidin-3-yl)methyl)phenyl)-3,8,9,10-tetrahydrocyclohepta[e]indole (16),
(E)-3-(6-(4-((1-(3-fluoropropyl)azetidin-3-yl)methyl)phenyl)-3,8,9,10-tetrahydrocyclohepta[e]indol-7-yl)prop-2-en-1-ol (17),
(E)-4-(6-(4-((1-(3-fluoropropyl)azetidin-3-yl)methyl)phenyl)-3,8,9,10-tetrahydrocyclohepta[e]indol-7-yl)-2-methylbut-3-en-2-ol (18),
(3-(6-(4-((1-(3-fluoropropyl)azetidin-3-yl)methyl)phenyl)-3,8,9,10-tetrahydrocyclohepta[e]indol-7-yl)phenyl)methanol (19),
(4-(6-(4-((1-(3-fluoropropyl)azetidin-3-yl)methyl)phenyl)-3,8,9,10-tetrahydrocyclohepta[e]indol-7-yl)phenyl)methanol (20),
2-(3-(6-(4-((1-(3-fluoropropyl)azetidin-3-yl)methyl)phenyl)-3,8,9,10-tetrahydrocyclohepta[e]indol-7-yl)phenyl)ethan-1-ol (21),
3-(6-(4-((1-(3-fluoropropyl)azetidin-3-yl)methyl)phenyl)-3,8,9,10-tetrahydrocyclohepta[e]indol-7-yl)benzoic acid (22),
4-(6-(4-((1-(3-fluoropropyl)azetidin-3-yl)methyl)phenyl)-3,8,9,10-tetrahydrocyclohepta[e]indol-7-yl)benzoic acid, 2,2,2-trifluoroacetic acid (23),
(E)-3-(6-(4-((1-(3-fluoropropyl)azetidin-3-yl)methyl)phenyl)-3,8,9,10-tetrahydrocyclohepta[e]indol-7-yl)acrylic acid (24),
3-(6-(4-((1-(3-fluoropropyl)azetidin-3-yl)methyl)phenyl)-3,8,9,10-tetrahydrocyclohepta[e]indol-7-yl)propan-1-ol (25),
7-(3-chloro-2-methylphenyl)-6-(4-((1-(3-fluoropropyl)azetidin-3-yl)oxy)phenyl)-3,8,9,10-tetrahydrocyclohepta[e]indole (26),
N-(4-(7-(3-chloro-2-methylphenyl)-3,8,9,10-tetrahydrocyclohepta[e]indol-6-yl)phenyl)-1-(3-fluoropropyl)azetidin-3-amine (27),
(S)—N-(4-(7-(3-chloro-2-methylphenyl)-3,8,9,10-tetrahydrocyclohepta[e]indol-6-yl)phenyl)-1-(3-fluoropropyl)pyrrolidin-3-amine (28), and
(Z)-7-(3-chloro-2-methylphenyl)-6-(4-((1-(3-fluoropropyl)pyrrolidin-3-ylidene)methyl)phenyl)-3,8,9,10-tetrahydrocyclohepta[e]indole (29).

14. A process for preparing a compound of formula (I) as described in anyone of claims 1 to 13, wherein a compound of formula 1D 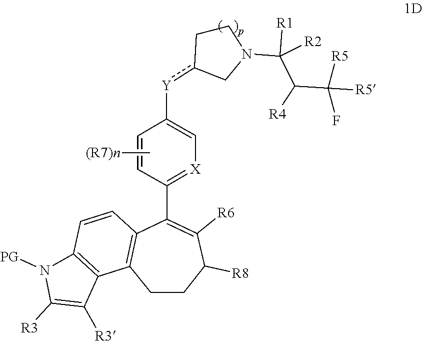
wherein R1, R2, R3, R3′, R4, R5, R5′, R6, R7, R8, n, p, X, [symbol] and Y are as defined in anyone of claims 1 to 12 and PG is a protecting group such as a tosyl group, a benzenesulfonamide group, a methoxymethylamine, a ethoxymethylamine or a 1-adamantyl carbamate group, is converted to a compound of formula (I), by a deprotection step, said deprotection step being optionally followed by a hydrogenation step with a catalyst, such as Pd/C or platinum oxide (PtO₂) under hydrogen (H₂) pressure to give the corresponding compound of formula (I) wherein [symbol] is a single bond,
said deprotection step being optionally preceded by a step of obtaining compound 1D, wherein either a compound of formula 1C 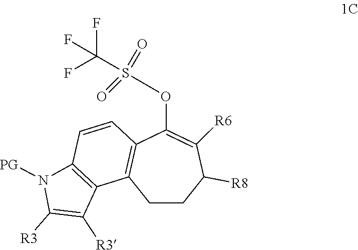
wherein R3, R3′, R6 and R8 are as defined above and PG is a protecting group such as defined above, or a compound 1G 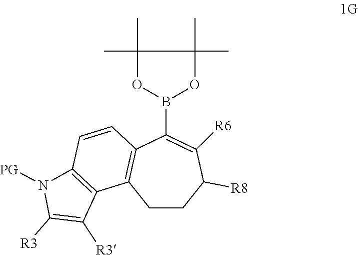
wherein R3, R3′, R6 and R8 are as defined above and PG is as defined above, is reacted in a Suzuki coupling step respectively with a compound 1E 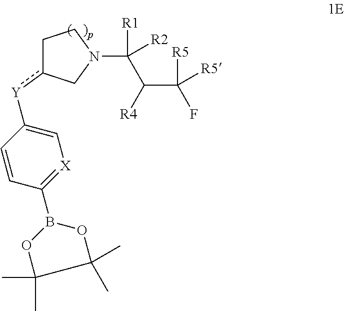
wherein R1, R2, R4, R5, R5′, R7, n, p, X, [symbol] and Y are as defined above
or with a compound 1H 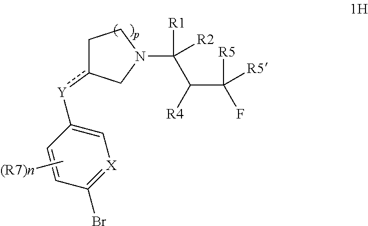
wherein R1, R2, R4, R5, R5′, R7, n, p, X, [symbol] and Y are as defined above, using a catalyst, for example [1,1′-bis(diphenylphosphino) ferrocene]dichloropalladium(II) (Pd(dppf)Cl₂) complex with DCM, in a solvent, for example in a mixture of dioxane and water and in the presence of a base, for example cesium carbonate (Cs₂CO₃), by heating up to reflux of solvent.

15. A process for preparing a compound of formula (I) as described in anyone of claims 1 to 13, wherein a compound of formula (I) as defined above, wherein [symbol] is a single bond, wherein alternatively a compound of formula 1D′ 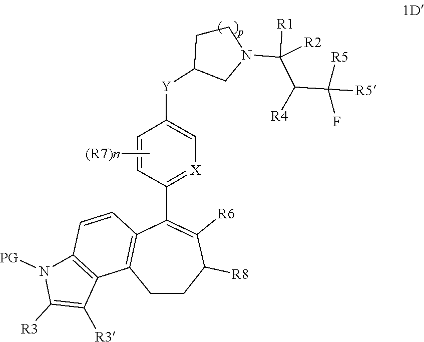
wherein R1, R2, R3, R3′, R4, R5, R5′, R6, R7, R8, n, p, X and Y are as defined in anyone of claims 1 to 12, and PG is a protecting group such as a tosyl group, a benzenesulfonamide group, a t-butyl carbamate group, a methoxymethylamine, a ethoxymethylamine or a 1-adamantyl carbamate group, is deprotected,
or a compound of formula 1S 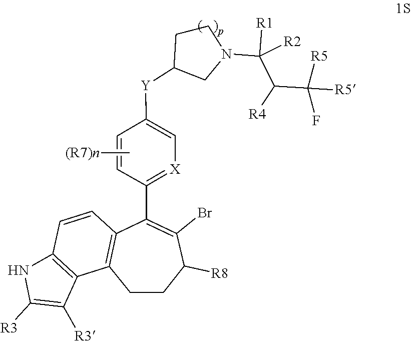
wherein R1, R2, R3, R3′, R4, R5, R5′, R7, R8, n, p, X and Y are as defined above, is reacted with a catalyst, such as [1,1′-bis(diphenylphosphino) ferrocene]dichloropalladium(II) (Pd(dppf)Cl₂) complex with DCM, in a solvent, such as a mixture of dioxane and water and in the presence of a base, for example cesium carbonate, by heating up to reflux of solvent,
said alternative steps being optionally preceded by a step of obtaining respectively a compound of formula 1D′ or a compound of formula 1S by respectively reacting a compound of formula 1Q 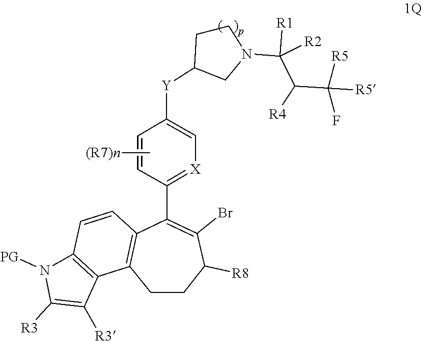
wherein R1, R2, R3, R3′, R4, R5, R5′, R6, R7, R8, n, p, X and Y are as defined above, and PG is a protecting group such as defined above,
with a boronic reagent R6B(OR′)₂, wherein —B(OR′)₂ is a boronic acid or a pinacolate ester and R6 is as defined above for obtaining said compound of formula 1D′,
or
by a deprotection step, in particular by treatment with an aqueous solution of potassium hydroxide in methanol, for obtaining said compound of formula 1S

16. Compounds selected from compounds of formula 1D, 1K, 1L, 1D′, 1O, 1Q, 1S, 1R, 1Y, 1Z, 2A and 2C, or any of its pharmaceutically acceptable salt, 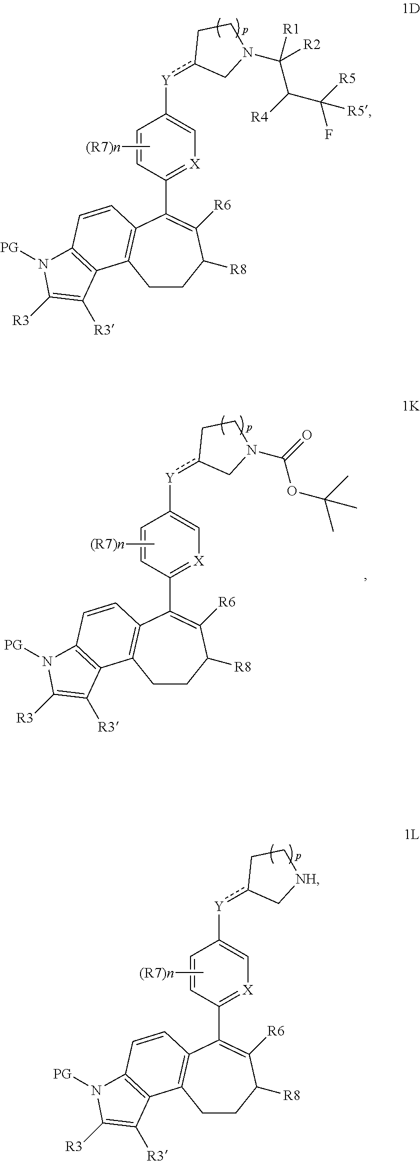 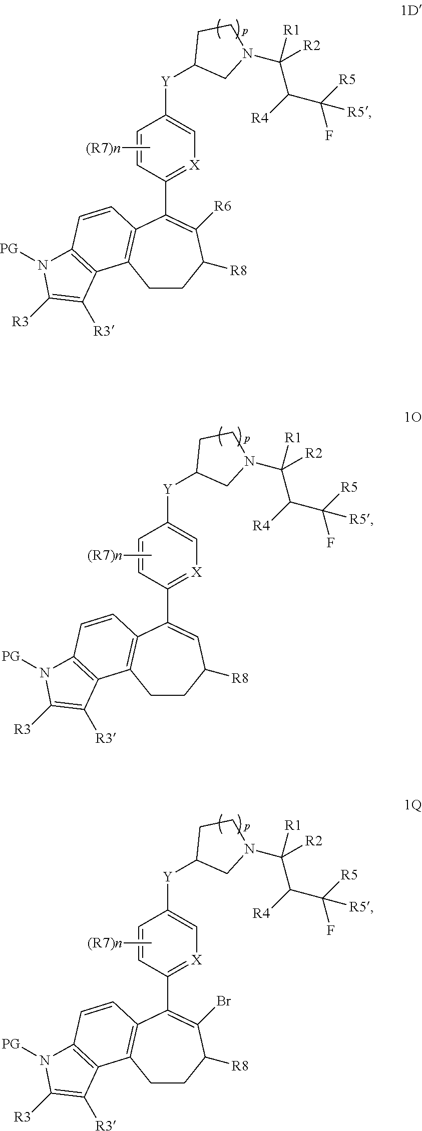 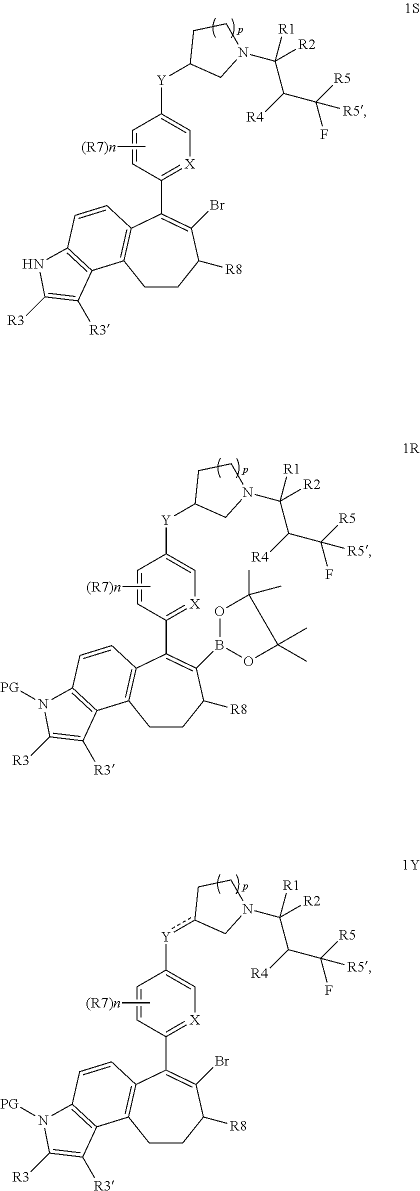 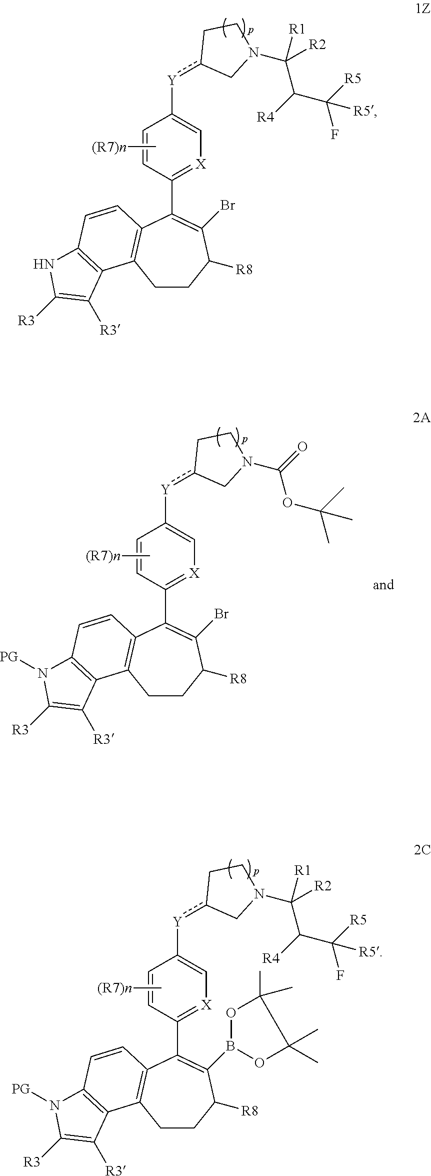
wherein R1, R2, R3, R3′, R4, R5, R5′, R6, R7, R8, n, p, X, [symbol] and Y are as defined in anyone of claims 1 to 12 and PG is a protecting group, such as a tosyl group, a benzenesulfonamide group, a t-butyl carbamate group, a methoxymethylamine group, a ethoxymethylamine group or a 1-adamantyl carbamate group, in particular a tosyl group.

17. A medicament, characterized in that it comprises a compound of formula (I) according to any of claims 1 to 13, or a pharmaceutically acceptable salt thereof.

18. A pharmaceutical composition, characterized in that it comprises a compound of formula (I) according to any of claims 1 to 13, or a pharmaceutically acceptable salt thereof, and at least one pharmaceutically acceptable excipient.

19. A compound of formula (I) according to any of claims 1 to 13, or a pharmaceutically acceptable salt thereof, for use as an inhibitor and degrader of estrogen receptors.

20. A compound of formula (I) according to any of claims 1 to 13, or a pharmaceutically acceptable salt thereof, for use in the treatment of ovulatory dysfunction, cancer, endometriosis, osteoporosis, benign prostatic hypertrophy or inflammation.

21. A compound of formula (I) for use according to claim 20, or a pharmaceutically acceptable salt thereof, for use in the treatment of cancer.
